SEQUENCE LISTING (11 sequences)

SEQ ID NO: 1 - DNA, 3463 bp, Homo sapiens
aatgaaacgaagcgctgaggaaagtggcttgggtttgaatattgtggttgagtctgaagcgctgggaggcggacattaaagtgaagtggttgcggtaacctggcctgggcctgaagtgagtgagaggcacatgaagagaagtattcaagtatttatacagataggaatcaagataatcaacaatgtctgtcactgaggaagacctgtgccaccatatgaaagtagtagttcgtgtacgtccggaaaacactaaagaaaaagcagctggatttcataaagtggttcatgttgtggataaacatatcctagtttttgatcccaaacaagaagaagtcagttttttccatggaaagaaaactacaaatcaaaatgttataaagaaacaaaataaggatcttaaatttgtatttgatgctgtttttgatgaaacgtcaactcagtcagaagtttttgaacacactactaagccaattcttcgtagttttttgaatggatataattgcacagtacttgcctatggtgccactggtgctgggaagacccacactatgctaggatcagctgatgaacctggagtgatgtatctaacaatgttacacctttacaaatgcatggatgagattaaagaagagaaaatatgtagtactgcagtttcatatctggaggtatataatgaacagattcgtgatctcttagtaaattcagggccacttgctgtccgggaagatacccaaaaaggggtggtcgttcatggacttactttacaccagcccaaatcctcagaagaaattttacatttattggataatggaaacaaaaacaggacacaacatcccactgatatgaatgccacatcttctcgttctcatgctgttttccaaatttacttgcgacaacaagacaaaacagcaagtatcaatcaaaatgtccgtattgccaagatgtcactcattgacctggcaggatctgagcgagcaagtacttccggtgctaaggggacccgatttgtagaaggcacaaatattaatagatcacttttagctcttgggaatgtcatcaatgccttagcagattcaaagagaaagaatcagcatatcccttacagaaatagtaagcttactcgcttgttaaaggattctcttggaggaaactgtcaaactataatgatagctgctgttagtccttcctctgtattctacgatgacacatataacactcttaagtatgctaaccgggcaaaggacattaaatcttctttgaagagcaatgttcttaatgtcaataatcatataactcaatatgtaaagatctgtaatgagcagaaggcagagattttattgttaaaagaaaaactaaaagcctatgaagaacagaaagccttcactaatgaaaatgaccaagcaaagttaatgatttcaaaccctcaggaaaaagaaatcgaaaggtttcaagaaatcctgaactgcttgttccagaatcgagaagaaattagacaagaatatctgaagttggaaatgttacttaaagaaaatgaacttaaatcattctaccaacaacagtgccataaacaaatagaaatgatgtgttctgaagacaaagtagaaaaggccactggaaaacgagatcatagacttgcaatgttgaaaactcgtcgctcctacctggagaaaaggagggaggaggaattgaagcaatttgatgagaatactaattggctccatcgtgtcgaaaaagaaatgggactcttaagtcaaaacggtcatattccaaaggaactcaagaaagatcttcattgtcaccatttgcacctccagaacaaagatttgaaagcacaaattagacatatgatggatctagcttgtcttcaggaacagcaacacaggcagactgaagcagtattgaatgctttacttccaaccctaagaaaacaatattgcacattaaaagaagccggcctgtcaaatgctgcttttgaatctgacttcaaagagatcgaacatttggtagagaggaaaaaagtggtagtttgggctgaccaaactgccgaacaaccaaagcaaaacgatctaccagggatttctgttcttatgacctttccacaacttggaccagttcagcctattccttgttgctcatcttcaggtggaactaatctggttaagattcctacagaaaaaagaactcggagaaaactaatgccatctcccttgaaaggacagcatactctaaagtctccaccatctcaaagtgtgcagctcaatgattctcttagcaaagaacttcagcctattgtatatacaccagaagactgtagaaaagcttttcaaaatccgtctacagtaaccttaatgaaaccatcatcatttactacaagttttcaggctatcagctcaaacataaacagtgataattgtctgaaaatgttgtgtgaagtagctatccctcataatagaagaaaagaatgtggacaggaggacttggactctacatttactatatgtgaagacatcaagagctcgaagtgtaaattacccgaacaagaatcactaccaaatgataacaaagacattttacaacggcttgatccttcttcattctcaactaagcattctatgcctgtaccaagcatggtgccatcctacatggcaatgactactgctgccaaaaggaaacggaaattaacaagttctacatcaaacagttcgttaactgcagacgtaaattctggatttgccaaacgtgttcgacaagataattcaagtgagaagcacttacaagaaaacaaaccaacaatggaacataaaagaaacatctgtaaaataaatccaagcatggttagaaaatttggaagaaatatttcaaaaggaaatctaagataaatcacttcaaaaccaagcaaaatgaagttgatcaaatctgcttttcaaagtttatcaataccctttcaaaaatatatttaaaatctttgaaagaagacccatcttaaagctaagtttacccaagtactttcagcaagcagaaaaatgaaactctttgttttcttcttttgtgttctaaaaaaataaaatttcaaaagaaaaggttgtcttttaagttttttaaatatttgttgccttttaaaatccctgagtgtaagttaccatggtggcagcttagttttactatgccacaacaagttgactaggacattttagtaaatggtagtgagttaaattatctttattattttttaaaaataagaatttagaagtggtaaaattatggcccaagatgtatttggttctctattatgttttgatacattattttaatcatatatatgactttccttttcaaaaatactttaatgtacaagtgtaaatatatgtgcccataaaatcattgtaaatattatttagtcatcacaaataaaatattgtcccttgctacttgatatattaaagatgtagattttaaagtgttt

SEQ ID NO: 2 - protein, 898 aa, Homo sapiens
MSVTEEDLCHHMKVVVRVRPENTKEKAAGFHKVVHVVDKHILVFDPKQEEVSFFHGKKTTNQNVIKKQNKDLKFVFDAVFDETSTQSEVFEHTTKPILRSFLNGYNCTVLAYGATGAGKTHTMLGSADEPGVMYLTMLHLYKCMDEIKEEKICSTAVSYLEVYNEQIRDLLVNSGPLAVREDTQKGVVVHGLTLHQPKSSEEILHLLDNGNKNRTQHPTDMNATSSRSHAVFQIYLRQQDKTASINQNVRIAKMSLIDLAGSERASTSGAKGTRFVEGTNINRSLLALGNVINALADSKRKNQHIPYRNSKLTRLLKDSLGGNCQTIMIAAVSPSSVFYDDTYNTLKYANRAKDIKSSLKSNVLNVNNHITQYVKICNEQKAEILLLKEKLKAYEEQKAFTNENDQAKLMISNPQEKEIERFQEILNCLFQNREEIRQEYLKLEMLLKENELKSFYQQQCHKQIEMMCSEDKVEKATGKRDHRLAMLKTRRSYLEKRREEELKQFDENTNWLHRVEKEMGLLSQNGHIPKELKKDLHCHHLHLQNKDLKAQIRHMMDLACLQEQQHRQTEAVLNALLPTLRKQYCTLKEAGLSNAAFESDFKEIEHLVERKKVVVWADQTAEQPKQNDLPGISVLMTFPQLGPVQPIPCCSSSGGTNLVKIPTEKRTRRKLMPSPLKGQHTLKSPPSQSVQLNDSLSKELQPIVYTPEDCRKAFQNPSTVTLMKPSSFTTSFQAISSNINSDNCLKMLCEVAIPHNRRKECGQEDLDSTFTICEDIKSSKCKLPEQESLPNDNKDILQRLDPSSFSTKHSMPVPSMVPSYMAMTTAAKRKRKLTSSTSNSSLTADVNSGFAKRVRQDNSSEKHLQENKPTMEHKRNICKINPSMVRKFGRNISKGNLR

SEQ ID NO: 3 - RNA, 19 nt, Artificial Sequence
  siRNA for targeting Kif18A sequence
gccaauucuucguaguuuu

SEQ ID NO: 4 - RNA, 18 nt, Artificial Sequence
  siRNA for targeting Kif18A sequence
gcagcuggauuucauaaa

SEQ ID NO: 5 - RNA, 21 nt, Artificial Sequence
  siRNA for targeting Kif18A sequence
  misc_feature (20)..(21): N at positions 20 and 21 is T
gccaauucuucguaguuuunn

SEQ ID NO: 6 - RNA, 21 nt, Artificial Sequence
  siRNA for targeting Kif18A sequence
  misc_feature (20)..(21): N at positions 20 and 21 is T
gcuggauuucauaaaguggnn

SEQ ID NO: 7 - DNA, 2862 bp, Homo sapiens
aaactgcggcggtttacgcggcgttaagacttcgtagggttagcgaaattgaggtttcttggtattgcgcgtttctcttccttgctgactctccgaatggccatggactcgtcgcttcaggcccgcctgtttcccggtctcgctatcaagatccaacgcagtaatggtttaattcacagtgccaatgtaaggactgtgaacttggagaaatcctgtgtttcagtggaatgggcagaaggaggtgccacaaagggcaaagagattgattttgatgatgtggctgcaataaacccagaactcttacagcttcttcccttacatccgaaggacaatctgcccttgcaggaaaatgtaacaatccagaaacaaaaacggagatccgtcaactccaaaattcctgctccaaaagaaagtcttcgaagccgctccactcgcatgtccactgtctcagagcttcgcatcacggctcaggagaatgacatggaggtggagctgcctgcagctgcaaactcccgcaagcagttttcagttcctcctgcccccactaggccttcctgccctgcagtggctgaaataccattgaggatggtcagcgaggagatggaagagcaagtccattccatccgaggcagctcttctgcaaaccctgtgaactcagttcggaggaaatcatgtcttgtgaaggaagtggaaaaaatgaagaacaagcgagaagagaagaaggcccagaactctgaaatgagaatgaagagagctcaggagtatgacagtagttttccaaactgggaatttgcccgaatgattaaagaatttcgggctactttggaatgtcatccacttactatgactgatcctatcgaagagcacagaatatgtgtctgtgttaggaaacgcccactgaataagcaagaattggccaagaaagaaattgatgtgatttccattcctagcaagtgtctcctcttggtacatgaacccaagttgaaagtggacttaacaaagtatctggagaaccaagcattctgctttgactttgcatttgatgaaacagcttcgaatgaagttgtctacaggttcacagcaaggccactggtacagacaatctttgaaggtggaaaagcaacttgttttgcatatggccagacaggaagtggcaagacacatactatgggcggagacctctctgggaaagcccagaatgcatccaaagggatctatgccatggcctcccgggacgtcttcctcctgaagaatcaaccctgctaccggaagttgggcctggaagtctatgtgacattcttcgagatctacaatgggaagctgtttgacctgctcaacaagaaggccaagctgcgcgtgctggaggacggcaagcaacaggtgcaagtggtggggctgcaggagcatctggttaactctgctgatgatgtcatcaagatgatcgacatgggcagcgcctgcagaacctctgggcagacatttgccaactccaattcctcccgctcccacgcgtgcttccaaattattcttcgagctaaagggagaatgcatggcaagttctctttggtagatctggcagggaatgagcgaggcgcggacacttccagtgctgaccggcagacccgcatggagggcgcagaaatcaacaagagtctcttagccctgaaggagtgcatcagggccctgggacagaacaaggctcacaccccgttccgtgagagcaagctgacacaggtgctgagggactccttcattggggagaactctaggacttgcatgattgccacgatctcaccaggcataagctcctgtgaatatactttaaacaccctgagatatgcagacagggtcaaggagctgagcccccacagtgggcccagtggagagcagttgattcaaatggaaacagaagagatggaagcctgctctaacggggcgctgattccaggcaatttatccaaggaagaggaggaactgtcttcccagatgtccagctttaacgaagccatgactcagatcagggagctggaggagaaggctatggaagagctcaaggagatcatacagcaaggaccagactggcttgagctctctgagatgaccgagcagccagactatgacctggagacctttgtgaacaaagcggaatctgctctggcccagcaagccaagcatttctcagccctgcgagatgtcatcaaggccttgcgcctggccatgcagctggaagagcaggctagcagacaaataagcagcaagaaacggccccagtgacgactgcaaataaaaatctgtttggtttgacacccagcctcttccctggccctccccagagaactttgggtacctggtgggtctaggcagggtctgagctgggacaggttctggtaaatgccaagtatgggggcatctgggcccagggcagctggggagggggtcagagtgacatgggacactccttttctgttcctcagttgtcgccctcacgagaggaaggagctcttagttacccttttgtgttgcccttctttccatcaaggggaatgttctcagcatagagctttctccgcagcatcctgcctgcgtggactggctgctaatggagagctccctggggttgtcctggctctggggagagagacggagcctttagtacagctatctgctggctctaaaccttctacgcctttgggccgagcactgaatgtcttgtactttaaaaaaatgtttctgagacctctttctactttactgtctccctagagatcctagaggatccctactgttttctgttttatgtgtttatacattgtatgtaacaataaagagaaaaaataaatcagctgtttaagtgtgtggaaa

SEQ ID NO: 8 - DNA, 7844 bp, Homo sapiens
actctaccccgcgccgtctcacggccccggccctagcttcaccccgactacccggcgtgcgcgtcctcctgccggcctgcaggcccggggcctccgcctgcttccccacagctgctccttgcggccccgcttgcgttcacgctgtcgcccgggccggcgcggccgcgggcaaccgctccccctcccacacctaccccgccccctccccgccttttccgccctccggtccccctccctcggcccgctgctgctgctccagatgaggtgatggcaacggccaacttcggcaagatccagatcgggatttacgtggagatcaagcgcagcgatggccgaatacatcaagcaatggtaacatctttaaatgaagataatgaaagtgtaactgttgaatggatagaaaatggagatacaaaaggcaaagagattgacctggagagcatcttttcacttaaccctgaccttgttcctgatgaagaaattgaacccagtccagaaacacctccacctccagcatcctcagccaaagtaaacaaaattgtaaagaatcgacggactgtagcttctattaagaatgaccctccttcaagagataatagagtggttggttcagcacgtgcacggcccagtcaatttcctgaacagtcttcctctgcacaacagaatggtagtgtttcagatatatctccagttcaagctgcaaaaaaggaatttggacccccttcacgtagaaaatctaattgtgtgaaagaagtagaaaaactgcaagaaaaacgagagaaaaggagattgcaacagcaagaacttagagaaaaaagagcccaggacgttgatgctacaaacccaaattatgaaattatgtgtatgatcagagactttagaggaagtttggattatagaccattaacaacagcagatcctattgatgaacataggatatgtgtgtgtgtaagaaaacgaccactcaataaaaaagaaactcaaatgaaagatcttgatgtaatcacaattcctagtaaagatgttgtgatggtacatgaaccaaaacaaaaagtagatttaacaaggtacctagaaaaccaaacatttcgttttgattatgcctttgatgactcagctcctaatgaaatggtttacaggtttactgctagaccactagtggaaactatatttgaaaggggaatggctacatgctttgcttatgggcagactggaagtggaaaaactcatactatgggtggtgacttttcaggaaagaaccaagattgttctaaaggaatttatgcattagcagctcgagatgtctttttaatgctaaagaagccaaactataagaagctagaacttcaagtatatgcaaccttctttgaaatttatagtggaaaggtgtttgacttgctaaacaggaaaacaaaattaagagttctagaagatggaaaacagcaggttcaagtggtgggattacaggaacgggaggtcaaatgtgttgaagatgtactgaaactcattgacataggcaacagttgcagaacatccggtcaaacatctgcaaatgcacattcatctcggagccatgcagtgtttcagattattcttagaaggaaaggaaaactacatggcaaattttctctcattgatttggctggaaatgaaagaggagctgatacttccagtgcggacaggcaaactaggcttgaaggtgctgaaattaataaaagccttttagcactcaaggagtgcatcagagccttaggtagaaataaacctcatactcctttccgtgcaagtaaactcactcaggtgttaagagattctttcataggtgaaaactctcgtacctgcatgattgccacaatctctccaggaatggcatcctgtgaaaatactcttaatacattaagatatgcaaatagggtcaaagaattgactgtagatccaactgctgctggtgatgttcgtccaataatgcaccatccaccaaaccagattgatgacttagagacacagtggggtgtggggagttcccctcagagagatgatctaaaacttctttgtgaacaaaatgaagaagaagtctctccacagttgtttactttccacgaagctgtttcacaaatggtagaaatggaagaacaagttgtagaagatcacagggcagtgttccaggaatctattcggtggttagaagatgaaaaggccctcttagagatgactgaagaagtagattatgatgtcgattcatatgctacacaacttgaagctattcttgagcaaaaaatagacattttaactgaactgcgggataaagtgaaatctttccgtgcagctctacaagaggaggaacaagccagcaagcaaatcaacccgaagagaccccgtgccctttaaaccggcatttgctgctaaaggatacccagaaccctcactactgtaacatacaacggttcagctgtaagggccatttgaaagtttggaattttaagtgtctgtggaaaatgttttgtccttcacctgaattacatttcaattttgtgaaacactcttttgtctacaaaatgcttctagtccaggaggcacaaccaagaactgggattaatgaagcattttgtttcatttacacaaatagtgatttacttttggagatccttgtcagttttattttctatttgatgaagtaagactgtggactcaatccagagccagatagtagggggaagccacagcatttccttttaactcagttcaatttttgtagtgagactgagcagttttaaatcctttgcgtgcatgcatacctcatcagtgattgtacataccttgcccactcctagagacagctgtgctcaccttttcctgctttgtgccttgattaaggctactgaccctaaatttctgaagcacagccaagaaaaattacattccttgtcattgtaaattacctttgtgtgtacatttttactgtatttgagacattttttgtgtgtgactagttaattttgcaggatgtgccatatcattgaacggaactaaagtctgtgacagtggatatagctgctggaccattccatcttatatgtaaagaaatctggaattattattttaaaaccatataacatgtgattataatttttcttagcattttctttgtaaagaactacaatataaactagttggtgtataataaaaagtaatgaaattctgagaagagttttatcttaggaaaatacatatatatgcagtgtgtgtgccagtgtggtattaacaagactaatagtgcagtttgatccttaccaatatcattacttaatttgaagtgttcattagcaccccaaaatataccttttctatgtactgttaaaagaaattggcttctgatgcatgaacatttacatgtacattgaaagtagtccataatagaagttagtttaagccaagtgtagacagtacattactcccttgaaaaagaattaagttgaaagagttgactttgccttaaaaggcagatctaacccaagctccatccagtaccaaatgtgaaacttcattgttgtttggtgagaatcgccaaattctcactaatacattggtatggtgtagggcatgctactttttaaacagcagcacttatttttacagattgctactccaaggaagaaaactggccacttttcatgtaaatattttgttcaaagatttgtatatctctctaggagttttccctcagttcccaggatggggtccaggagtagattaacagctaaaaatctcccaaggatatcttgttttgatttatttactccagggaactatcagctccttcacaggagccaaaggggagctatgaatagagggtcacatgagccagattctttactgttcataaaacccagagagtagttgtgaaagatcctaatacaattgtgaaaagctctgtaaacatgaaatctaaaacaaatgtagattttcacaatatgcttattaataaatgaagtctatggaataagttttagagataatttacttcagtcacctttgttttggaaaggactcaggttttcttgcagctgtaagatatattctatttgtgtttatttcaaagggaagaggaaggatggagaactgttacaattgaccttgcaaaggatatttaaaaaaaaggtagtttttgagattaaccaactttcaaagggcaataaagacatgtgaatttgctcattttaaagcacaacagatgattagcttcaaatttattatttgcattggatgggtactgtctttggaaagtctccttatagacaaatatgctgccttacactatgatggcttcattctgatcaggtattttaaaaattagtaccagaaaagatactggaggtaatataatacagtccttcagctttacagatagtgaaaactgaaggccagaaaaggaactaaaactcagcagttcataggggtagagggaaataacctgagaaagccgagttaaatcttaaaatttttactataaggatagagatgatacaagtgaactctgcaaaatagttttgtgaaattaaacaaaaaaatctactcttaatgtatattatttcatatttgtttaacaaaagcagcttgatgcctttgttctggattatacattaagaacaagcattattttaattatgtagtaacatttactatgactttgaagccagatttcaaaatctggctttgttaaattaattggaaacactcttctattaagttaggtattaaaaaaagccaaatatcaataaagatatttttattaattttttatagaaacaaaatagctactataactctattatagtatattaacagcacttagtattatattgacttaaacatagtagctaataaaactagtttatgacatctattgaaattctcagtctgacagttttaaaatagcttaaaactaagcaccaggtatatgaaaaaggaactggaaattttaattgtccagaaatcataggaatactgggaacctgtagttatgggaaatggctgtttttctgaagttgaaaccagtttcaattacaaaaccctctgaaattttgctaagcctatagttatctccctaagaaccataaaaaaagataattttattgttaatttcattccagtttagcttcctcattttacatgttgggagtcatgccctttaatatgcctggcactgcacggtggcagtagcagacttgggcttacccagaaccctaagtctctgacgctataggatagcgaaagaggtaggcaatcgtaacagacactgtataggataaatagggaagagaatgagagaaccaaaataaaaataatttaagaaacaattgttttactgtacatgtgaataacaagaaatggtacggggaatgtgaataacacgaaatggtatggggaatgtgtgactagtgaagcatattttaacttttttattactggaggaacagagctaaaggcctaaagaaggccttacagtatataacagtaaatagaagagtaacatctttatacaataaactgttagaaatgataagtgtaaagttgtgcgtctctgcggctcctgaacttgaagattattatgaatagattggaccagcattatatattaaaaactttgatacttagaactttccaactttaaaattcagaatcataaatggtgacaacagtagtagtattgaaccaaaaatagtcaagtaaataatgtctcagtaaagcaaaagcattatcttctcaaatacaaaaaatacaaaattcatttcttttcttgaccttgaaaatttctgttttccaaatacctaggaaaaatgaataccttctgcgttgaatccatgtagcaatctgaaaaaagaaatcaaaatggaatggtactgaaaagctaatttgtagcacataacggtatatagttctataccattaatactaaaacatgaatacagcatgcttacagagcccacccactcctaatactagttattaaagaaacgttactggctgggcgcagtggcttatgcctgtaatcccagcactttgggaggctgaggcgggtggattacctgaggtcagtagttcgagaacagtctggccaacatggtgaaaccccgtctctactaaaaatacaaaaattagcctggcatggtggcacatgcctgtaatcccagctactcgggtggttgaggcaggagatttgcctgaacccaggaggcggaggttgcagtgagctgagattgtgccactggagtccagcctgggtgacagagcaagactctgtctcaaaaaaaaaaaaaaaagaaatgttattgtctgactaaatttataagatatgtattggttaaaaccatgccatgttggtgcctctttcattagagcctttatgtcgtaatgaatttgactaaaattatcttagactttcattatcccaggcctaagaactcactggcatttgtttcaaggtaactgaacaagaagctgtttggaattggcagaagtcagatgaaaaaccaatcttacatgccccttcttcctctcttgagctgttgtttatattcaaattaaatacacattgtttctctctgtagatacctatgtacttaatagattctagttagtaaactgcacatgcccaataactttgaggaaatttagtgaaaatgaagaaaaagagaaaatatttctctttagacctgaggttatgtttaggctggcccatagaaacaggtccagataaatttctaaaaaagcaaagtagatatttatgaatagtattcaatgcctaggattaacatctaaaatgactcagtagtactgctagccagccaataaaatataaactccatttgtcttagttatatagaactgtgtttccagcttagaaaaagtcaaaccaatgacttttagaacaatctactctcattttttattcagcctctagaacatggaagctttaaaagtgaattggctaaataggcaagaccttctgaaagttaacatcttaatgattaaaaacagtaagtacaggttagtaattacctgggtaattaattgaagccttattctgttttcataagacttacttgcttaattcaagcaaaacaaattttggtctaaattacctagataattatgacagctttttacttgagaagtgtagaacttgcttcaggctacaaaactgtattattcctaaatggataaccaggtaggattctaactggcattattgtatgcttaagattgatttaacaacagctattcccagtaaggaaattttaaaaatcagatccagttacatgtattatgatttttctaccttatggactattttggagggataagctattaagactaagactatgaatgagagttggggaaggagcaggaagggaggaacctgcacaccacattggaacctgcacaccacattaacacaaaggcaatcttctggctcggactgttctttactactgttcttaaagaaaatgttcattctgctgcagctaactagcctccatcttctacaccaaatactattccatgccatggaagtgctatgcaataactctcccaggtagcaccttataccgcttaaaagcctttaaaatctccaatctgaaggtgtcacagtaaagaaatgtaaacacttaggaaaacaaaaatgtaattacctgatgaagtcatctatgtccatggaacgggcccgtttgtcactaaaacctgtgctggttaggatttgctgtattttatctgctatgctgaaatcttctggtattatctatcaatataagattcagaataaatgaacgacatatctttaa

SEQ ID NO: 9 - DNA, 2335 bp, Homo sapiens
gtagtggccccagtccgggccccggcgcgctaggctcacaaaggcaggcacagactgcaaccctgctcagtgctccgggcgcttcaggctggcttgggtcctgctgctccaaccccaagggccctggagcgctccctgatacctccatcactcaccatggccagccagttctgcctccctgaatccccatgtctctcgcccctgaaacccttgaagccacatttcggagacatccaagagggcatctacgtggcgatccagcgcagtgacaagcggatccacctcgctgtggtcacggagatcaacagagaaaactattgggtcacggtagagtgggtggagaaagcagtcaaaaaaggcaagaagattgacctggagaccatactcctgctgaatccagctctggactctgctgaacaccccatgccgcccccgcccttatcccccttggctctggcgccctcttcggccatcagggaccagcgtaccgccacgaaatgggttgcgatgatcccccagaaaaaccaaacagcctcaggggacagcctggatgtgagggtccccagcaaaccttgtctgatgaagcagaaaaagtctccctgcctctgggaaatccagaaactgcaggagcagcgggaaaagcgcaggcggctgcagcaggagatccgagctagacgcgccctcgatgtcaataccagaaaccccaactacgaaatcatgcacatgatcgaagagtatcgcaggcacctggacagcagcaagatctcagtcctggagcccccgcaagaacatcgcatctgcgtctgcgtgaggaagcggcctctcaaccagcgagagacaaccttaaaggacctggatatcatcaccgtcccctcggacaatgtggttatggtgcatgagtccaagcaaaaggtggacctcactcgctacctgcagaaccagaccttctgcttcgaccatgccttcgatgacaaagcctccaacgagttggtgtaccagttcaccgcccagccactggtggagtccatcttccgcaagggcatggccacctgctttgcctatgggcagacgggaagtgggaagacgtacaccatgggtggagacttttcaggaacggcccaagattgttctaagggcatttatgctctggtggcacaggatgtctttctcctgctcagaaactccacatatgagaagctggacctcaaagtctatgggacattttttgagatttatgggggcaaggtgtatgatttgttgaactggaagaagaagctgcaagtccttgaggatggcaatcagcaaatccaagtggtcgggctgcaggagaaagaggtgtgttgtgtggaggaagtgctgaacctggtggaaatagggaatagctgtcggacttccaggcaaacacctgtcaacgctcactcatccaggagccatgcagtgttccagatcatcctgaagtcaggacggataatgcatggcaagttttccctcgttgatttagctgggaatgaaagaggagcagatacaaccaaggccagccggaaaaggcagctggaaggggcagagattaacaagagtcttctagccctcaaagaatgtattctggctttgggtcagaacaagcctcacaccccattcagagccagcaaactcacactggtgctccgggactcctttataggccagaactcctccacttgcatgattgctaccatctctccggggatgacctcttgtgaaaacactctcaacactttaagatatgcaaacagagtaaaaaaattaaatgtagatgtaaggccctaccatcgtggccactatccgattggacatgaggcaccaaggatgttaaaaagtcacatcggaaattcagaaatgtcccttcagagggatgaatttattaaaataccttatgtacagagtgaggagcagaaagagattgaagaggttgaaacattacccactctgttagggaaggataccacaatttcagggaagggatctagccaatggctggaaaacatccaggagagagctggtggagtacaccatgatattgatttttgcattgcccggtctttgtccattttggagcagaaaattgatgctctgaccgagatccaaaagaaactgaaattattactagctgacctccacgtgaagagcaaggtagagtgaagccaatggcgagagatcaggtccgaaatgctgcattgctgcagtttccaccactcttatacaggaaaactgtccaaattatctaaagatcctcctgagaagcttaaaacatcttaaaatacactgatgggaaacatgctctttcttctgcctctgt

SEQ ID NO: 10 - DNA, 1219 bp, Homo sapiens
agttgtttgcgggcggccgggagagtagcagtgccttggaccccaggatggcccagaaggagaactcctacccctggccctacggccgacagacggctccatctggcctgagcaccctgccccagcgagtcctccggaaagagcctgtcaccccatctgcacttgtcctcatgagccgctccaatgtccagcccacagctgcccctggccagaaggtgatggagaatagcagtgggacacccgacatcttaacgcggcacttcacaattgatgactttgagattgggcgtcctctgggcaaaggcaagtttggaaacgtgtacttggctcgggagaagaaaagccatttcatcgtggcgctcaaggtcctcttcaagtcccagatagagaaggagggcgtggagcatcagctgcgcagagagatcgaaatccaggcccacctgcaccatcccaacatcctgcgtctctacaactatttttatgaccggaggaggatctacttgattctagagtatgccccccgcggggagctctacaaggagctgcagaagagctgcacatttgacgagcagcgaacagccacgatcatggaggagttggcagatgctctaatgtactgccatgggaagaaggtgattcacagagacataaagccagaaaatctgctcttagggctcaagggagagctgaagattgctgacttcggctggtctgtgcatgcgccctccctgaggaggaagacaatgtgtggcaccctggactacctgcccccagagatgattgaggggcgcatgcacaatgagaaggtggatctgtggtgcattggagtgctttgctatgagctgctggtggggaacccaccctttgagagtgcatcacacaacgagacctatcgccgcatcgtcaaggtggacctaaagttccccgcttccgtgcccatgggagcccaggacctcatctccaaactgctcaggcataacccctcggaacggctgcccctggcccaggtctcagcccacccttgggtccgggccaactctcggagggtgctgcctccctctgcccttcaatctgtcgcctgatggtccctgtcattcactcgggtgcgtgtgtttgtatgtctgtgtatgtataggggaaagaagggatccctaactgttcccttatctgttttctacctcctcctttgtttaataaaggctgaagctttttgtactca

SEQ ID NO: 11 - DNA, 2466 bp, Homo sapiens
gaattctaacggctgagctcttggaagacttgggtccttgggtcgcaggtgggagccgacgggtgggtagaccgtgggggatatctcagtggcggacgaggacggcggggacaaggggcggctggtcggagtggcggagcgtcaagtcccctgtcggttcctccgtccctgagtgtccttggcgctgccttgtgcccgcccagcgcctttgcatccgctcctgggcaccgaggcgccctgtaggatactgcttgttacttattacagctagagggtctcactccattgcccaggccagagtgcggggatatttgataagaaacttcagtgaaggccgggcgcggtggctcatgcccgtaatcccagcattttcggaggccgaggctggagtgcaatggtgtgatctcagctcactgcaacctctgcttcctgggtttaagtgattctcctgcctcagcctcccgagtagctgggattacaggcatcatggaccgatctaaagaaaactgcatttcaggacctgttaaggctacagctccagttggaggtccaaaacgtgttctcgtgactcagcaatttccttgtcagaatccattacctgtaaatagtggccaggctcagcgggtcttgtgtccttcaaattcttcccagcgcattcctttgcaagcacaaaagcttgtctccagtcacaagccggttcagaatcagaagcagaagcaattgcaggcaaccagtgtacctcatcctgtctccaggccactgaataacacccaaaagagcaagcagcccctgccatcggcacctgaaaataatcctgaggaggaactggcatcaaaacagaaaaatgaagaatcaaaaaagaggcagtgggctttggaagactttgaaattggtcgccctctgggtaaaggaaagtttggtaatgtttatttggcaagagaaaagcaaagcaagtttattctggctcttaaagtgttatttaaagctcagctggagaaagccggagtggagcatcagctcagaagagaagtagaaatacagtcccaccttcggcatcctaatattcttagactgtatggttatttccatgatgctaccagagtctacctaattctggaatatgcaccacttggaacagtttatagagaacttcagaaactttcaaagtttgatgagcagagaactgctacttatataacagaattggcaaatgccctgtcttactgtcattcgaagagagttattcatagagacattaagccagagaacttacttcttggatcagctggagagcttaaaattgcagattttgggtggtcagtacatgctccatcttccaggaggaccactctctgtggcaccctggactacctgccccctgaaatgattgaaggtcggatgcatgatgagaaggtggatctctggagccttggagttctttgctatgaatttttagttgggaagcctccttttgaggcaaacacataccaagagacctacaaaagaatatcacgggttgaattcacattccctgactttgtaacagagggagccagggacctcatttcaagactgttgaagcataatcccagccagaggccaatgctcagagaagtacttgaacacccctggatcacagcaaattcatcaaaaccatcaaattgccaaaacaaagaatcagctagcaaacagtcttaggaatcgtgcagggggagaaatccttgagccagggctgccatataacctgacaggaacatgctactgaagtttattttaccattgactgctgccctcaatctagaacgctacacaagaaatatttgttttactcagcaggtgtgccttaacctccctattcagaaagctccacatcaataaacatgacactctgaagtgaaagtagccacgagaattgtgctacttatactggttcataatctggaggcaaggttcgactgcagccgccccgtcagcctgtgctaggcatggtgtcttcacaggaggcaaatccagagcctggctgtggggaaagtgaccactctgccctgaccccgatcagttaaggagctgtgcaataaccttcctagtacctgagtgagtgtgtaacttattgggttggcgaagcctggtaaagctgttggaatgagtatgtgattctttttaagtatgaaaataaagatatatgtacagacttgtattttttctctggtggcattcctttaggaatgctgtgtgtctgtccggcaccccggtaggcctgattgggtttctagtcctccttaaccacttatctcccatatgagagtgtgaaaaataggaacacgtgctctacctccatttagggatttgcttgggatacagaagaggccatgtgtctcagagctgttaagggcttatttttttaaaacattggagtcatagcatgtgtgtaaactttaaatatgcaaataaataagtatctatgtc

(19) United States
(12) Patent Application Publication
(10) Pub. No.: US 2023/0233565 A1
(43) Pub. Date: Jul. 27, 2023

(54) A TREATMENT APPROACH INVOLVING KIF18A INHIBITION FOR CHROMOSOMALLY UNSTABLE TUMORS

(71) Applicant: UNIVERSITY OF VERMONT, Burlington, VT (US)

(72) Inventor: Jason STUMPFF, Shelburne, VT (US)

(21) Appl. No.: 17/924,809

(22) PCT Filed: May 11, 2021
(86) PCT No.: PCT/US2021/031761

§ 371 Date: Nov. 11, 2022

Related U.S. Application Data
(60) Provisional application No. 63/022,885, filed on May 11, 2020.

(51) Int. Cl. A61K 31/519; A61P 35/00; A61K 31/444; A61K 31/5377; A61K 31/4545; A61K 31/475; A61K 31/4745; A61K 31/165; A61K 31/4184; A61K 31/357

(52) U.S. Cl. CPC A61K 31/519; A61P 35/00; A61K 31/444; A61K 31/5377; A61K 31/4545; A61K 31/475; A61K 31/4745; A61K 31/165; A61K 31/4184; A61K 31/357

(57) ABSTRACT

The present application is directed to a method of inhibiting proliferation of chromosome instable cancer cells. This method involves administering, to a population of cancer cells comprising chromosome instable cancer cells, an inhibitor of Kinesin Family Member 18A (KIF18A) at a dosage effective to inhibit proliferation of said chromosome instable cancer cells. The inhibitors of KIF18A may also be used in a method treating cancer in a subject. This method involves selecting a subject having cancer, where the cancer is characterized by chromosomal instability, and administering to the subject an inhibitor of KIF18A at a dosage effective to treat the cancer in the subject. Also disclosed is a combination therapeutic including an inhibitor of Kinesin Family Member 18A (KIF18A) and agent that promotes microtubule turnover or a cyclin-dependent kinase (CDK) inhibitor.

This application claims the benefit of U.S. Provisional Patent Application Ser. No. 63/022,885, filed May 11, 2020, which is hereby incorporated by reference in its entirety.

This invention was made with government support under Grant No. 5R01GM121491 awarded by the National Institutes of Health. The government has certain rights in the invention.

### FIELD

The present application is directed to methods of inhibiting proliferation of chromosome instable cancer cells that involve Kinesin Family Member 18A (KIF18A) inhibition. Methods of treating cancer and combinatorial approaches and therapeutics are also disclosed.

### BACKGROUND

Genetic instability is a common feature of tumor cells, and a large number of tumor cells exhibit frequent loss or gain of chromosomes (Lengauer et al., “Genetic Instabilities in Human Cancers,” Nature 396:643-649 (1998)). This chromosomal instability (CIN) is primarily attributable to defects leading to abnormal interactions between chromosomes and mitotic spindle microtubules, which in turn increase chromosome segregation errors (Bakhoum et al., “Deviant Kinetochore Microtubule Dynamics Underlie Chromosomal Instability,” Curr Biol 19:1937-1942 (2009); Ertych et al., “Increased Microtubule Assembly Rates Influence Chromosomal Instability in Colorectal Cancer Cells,” Nat Cell Biol 16:779-791 (2014); Cimini et al., “Merotelic Kinetochore Orientation is a Major Mechanism of Aneuploidy in Mitotic Mammalian Tissue Cells,” The Journal of Cell Biology 153:517-527 (2001); Bakhoum et al., “Genome Stability is Ensured by Temporal Control of Kinetochore-microtubule Dynamics,” Nat Cell Biol 11:27-35 (2009); Ganem et al., “A Mechanism Linking Extra Centrosomes to Chromosomal Instability,” Nature 460:278-282 (2009); Bakhoum Et Al., “The Mitotic Origin of Chromosomal Instability,” Curr Biol 24:R148-R149 (2014)). While CIN contributes to tumor progression, heterogeneity, drug resistance, and metastasis, it has been proposed that the same properties driving instability could provide an Achilles' heel for CIN cell-specific targeted therapies (Lengauer et al., “Genetic Instabilities in Human Cancers,” Nature 396:643-649 (1998); Lee et al., “Chromosomal Instability Confers Intrinsic Multidrug Resistance,” Cancer Res 71:1858-1870 (2011); Bakhoum et al., “Chromosomal Instability Drives Metastasis Through a Cytosolic DNA Response,” Nature 553:467-472 (2018)). Compared to chromosomally stable cells, CIN cells display increased spindle microtubule polymerization and reduced turnover of the attachments between spindle microtubules and kinetochores, which are specialized protein structures that assemble at the centromeric regions of mitotic chromosomes (Bakhoum et al., “Deviant Kinetochore Microtubule Dynamics Underlie Chromosomal Instability,” Curr Biol 19:1937-1942 (2009); Ertych et al., “Increased Microtubule Assembly Rates Influence Chromosomal Instability in Colorectal Cancer Cells,” Nat Cell Biol 16:779-791 (2014)). Thus, CIN cells may be particularly vulnerable to anti-mitotic therapies that target the microtubule cytoskeleton.

Consistent with this idea, microtubule-targeting agents are effective therapeutics for a wide variety of tumors (Jordan & Wilson, “Microtubules as a Target for Anticancer Drugs,” Nat Rev Cancer 4:253-265 (2004)). Paclitaxel, a microtubule stabilizing drug routinely utilized to treat solid tumors, was originally demonstrated to induce cytotoxicity by preventing cells from completing mitosis (Weaver, B. A. A., “How Taxol/paclitaxel Kills Cancer Cells,” Mol Biol Cell 25:2677-2681 (2014)). However, due to adverse side effects associated with the broad inhibition of microtubule function, significant effort has been made to identify mitotic regulators that could be targeted with lower toxicity in cancer patients. While drugs targeting mitotic proteins that are required to complete cell division have shown promise in preclinical models, they have been largely unsuccessful in clinical trials (Tischer & Gergely, “Anti-mitotic Therapies in Cancer,” J Cell Biol 526:jcb.201808077 (2018)). One explanation for the apparent paradox presented by failed mitotic targeting strategies and the effective therapeutic results seen with paclitaxel is suggested by work demonstrating that clinically relevant paclitaxel doses induce abnormal, multipolar divisions in tumors, rather than preventing mitotic division altogether (Zasadil et al., “Cytotoxicity of Paclitaxel in Breast Cancer is Due to Chromosome Missegregation on Multipolar Spindles,” Sci Transl Med 6:229ra43-229ra43 (2014); Weaver, B. A. A., “How Taxol/paclitaxel Kills Cancer Cells,” Mol Biol Cell 25:2677-2681 (2014)). Thus, efforts to mimic the effects of paclitaxel on mitotic cells need to be refocused towards identifying proteins that can be targeted to disrupt normal bipolar divisions, ideally in a tumor cell specific manner.

The present application is directed to overcoming these and other deficiencies in the art.

### SUMMARY

A first aspect of the present disclosure is directed to a method of inhibiting proliferation of chromosome instable cancer cells. This method involves administering, to a population of cancer cells comprising chromosome instable cancer cells, an inhibitor of Kinesin Family Member 18A (KIF18A) at a dosage effective to inhibit proliferation of said chromosome instable cancer cells.

A related aspect of the present disclosure is directed to a method of treating cancer in a subject, this method involves administering to a subject having cancer, wherein said cancer is characterized by chromosomal instability, an inhibitor of KIF18A at a dosage effective to treat the cancer in the subject.

Another aspect of the present disclosure is directed to a combination therapeutic comprising an inhibitor of Kinesin Family Member 18A (KIF18A), and an agent that promotes microtubule turnover to the population of cells.

A further aspect of the present disclosure is directed to combination therapeutic comprising an inhibitor of Kinesin Family Member 18A (KIF18A) and a cyclin-dependent kinase (CDK) inhibitor.

Chromosomal instability (CIN), characterized by frequent missegregation of chromosomes during mitosis, is a hallmark of tumor cells caused by changes in the dynamics and control of microtubules that comprise the mitotic spindle (Lengauer et al., “Genetic Instabilities in Human Cancers,” Nature 396:643-649 (1998); Bakhoum et al., “Deviant Kinetochore Microtubule Dynamics Underlie Chromosomal Instability,” Curr Biol 19:1937-1942 (2009); Ertych et al., “Increased Microtubule Assembly Rates Influence Chromosomal Instability in Colorectal Cancer Cells,” Nat Cell Biol 16:779-791 (2014), which are hereby incorporated by reference in their entirety). Thus, CIN tumor cells may respond differently than normal diploid cells to treatments that target mitotic spindle regulation. This idea was tested by inhibiting a subset of kinesin motor proteins that control spindle microtubule dynamics and mechanics but are not required for the proliferation of near-diploid cells. The results indicate that KIF18A is required for proliferation of CIN cells derived from triple negative breast cancer or colorectal cancer tumors but not normal breast epithelial cells or near-diploid colorectal cancer cells exhibiting microsatellite instability. CIN tumor cells exhibit mitotic delays, multipolar spindles due to centrosome fragmentation, and increased cell death following inhibition of KIF18A. These mitotic defects were further enhanced by increasing the activity of the microtubule depolymerizing kinesin KIF2C/MCAK and are reminiscent of the phenotypes that result from clinically relevant doses of the chemotherapeutic drug paclitaxel (Zasadil et al., “Cytotoxicity of Paclitaxel in Breast Cancer is Due to Chromosome Missegregation on Multipolar Spindles,” Sci Transl Med 6:229ra43-229ra43 (2014), which is hereby incorporated by reference in its entirety). The results indicate that the altered spindle microtubule dynamics characteristic of CIN tumor cells can be exploited to reduce their proliferative capacity.

### BRIEF DESCRIPTION OF THE DRAWINGS

FIGS. 1A-1D show the kinetic cell proliferation assay validation. FIG. 1A is an example trace of MDA-MD-231 cell density (cells/mm²) as a function of time over 96 hours. FIG. 1B is representative images of HCT116 cells showing the masks created by Gen5 software for automated cell counting. FIGS. 1C-1D are scatterplots of automated (FIG. 1C) LS1034 and (FIG. 1D) HCT116 cell counts using high-contrast brightfield microscopy as a function of cell counts of the same fields using a nuclear dye (Hoechst). Linear correlation indicates consistency in automated cell counting across different cell densities.

FIGS. 2A-2F show kinesins are effectively depleted by siRNA in breast and colorectal cell lines. FIG. 2A are Western blots showing siRNA knockdown (KD) efficiencies for the indicated kinesins in TNBC and diploid breast epithelial cells. FIG. 2B shows immunofluorescence images demonstrating efficiency of KIF18B KD in TNBC and diploid breast epithelial cells. Scale bar is 10 microns. FIG. 2C are graphs showing the quantification of kinesin knockdowns in TNBC and diploid breast epithelial cells. Relative remaining protein indicates the proportion of each kinesin remaining in cells after siRNA knockdown (measured via Western blot or immunofluorescence) relative to control. FIG. 2D are immunofluorescence images demonstrating efficiency of KIF18A siRNA-mediated knockdown in CRC cell lines. Scale bar is 10 microns. FIG. 2E is a graph showing the quantification of kinesin knockdowns in CRC cell lines. The relative remaining protein was measured via immunofluorescence, and all values within each cell line were normalized to control. FIG. 2F is a graph showing the quantitative PCR measurements of KIF18A mRNA levels after siRNA-mediated knockdown in diploid breast epithelial cells and one TNBC cell line. All graphs show mean+/−SD.

FIGS. 3A-3C demonstrate KIF18A is required for the proliferation of chromosomally unstable cells. FIG. 3A is plots of fold change in cell density (cells/mm²) after 96 hours in the indicated cell lines following knockdown (KD) of kinesin proteins. Data are normalized to cells treated with control siRNA. FIG. 3B shows representative images of MDA-MB-231 and MCF10A cells treated with either control or KIF18A siRNA. Scale bars are 100 microns. FIG. 3C shows plots of normalized fold change in cell density (cells/mm²) of MSI and CIN colorectal cancer cell lines after 96 hours. At least 24 wells from three independent experiments were analyzed in A and C. All graphs show mean+/−SD. **** p<0.0001, *** p<0.001, ** p<0.01, * p<0.05.

FIGS. 4A-4D show KIF18A depletion increases cell death in CIN cells. FIG. 4A is representative images of HT29 and MCF10A cells labeled with Celltox Green cytotoxicity dye five days after siRNA transfection. Scale bars are 100 microns. FIG. 4B is a graph of relative cell death calculated as the normalized ratio of the change in Celltox-stained cell density to the change in total cell density over 96 hours. A total of at least 68 wells from three independent experiments were analyzed. FIG. 4C is a graph of relative expression of cleaved-caspase 3 measured via Western blot for each condition. Results are from three independent experiments. FIG. 4D is a Western blot showing representative cleaved-caspase 3 (CC3) expression levels. All graphs show mean+/−SD. **** p<0.0001, *** p<0.001, ** p<0.01, * p<0.05

FIGS. 5A-5F show KIF18A depletion causes mitotic arrest in CIN cancer cells. FIGS. 5A-5B are representative images of (FIG. 5A) HT29 cells or (FIG. 5B) HCT116 cells treated with control or KIF18A siRNAs. Scale bars are 10 microns. FIG. 5C is a graph showing the percentage of mitotic cells (mitotic index) observed in fixed populations of control or KIF18A siRNA-treated CRC cells. At least 60 fields from three independent experiments were analyzed per condition. FIG. 5D is a graph of the time between nuclear envelope breakdown (NEB) and anaphase onset (AO) in control or KIF18A siRNA-treated cells. At least 150 cells from three independent experiments were analyzed per condition. Cell types most sensitive to KIF18A KD contained a significant subpopulation of cells that failed to complete mitosis during imaging studies and were arrested for up to 20 hours. FIG. 5E is a graph of the percentage of control or KIF18A siRNA-treated cells that entered mitosis at least 200 minutes before the end of the movie but did not divide. FIG. 5F shows frames from DIC live cell imaging of HT29 and MCF10A cells treated with control or KIF18A siRNA, showing progression from NEB to AO. Scale bars are 5 microns. All graphs show mean+/−SD. **** p<0.0001, *** p<0.001, ** p<0.01, * p<0.05.

FIGS. 6A-6B show KIF18A KD increases the percentage of cells in mitosis for TNBC cells, but not for diploid breast epithelial cells. FIG. 6A shows graphs of the percent of cells in mitosis, as determined from fixed cell images, 48 hours after siRNA-mediated knockdown (KD) of the specified kinesins. FIG. 6B shows representative images of MDA-MB-231 cells treated with either control or KIF18A siRNA. Scale bar is 10 microns. All graphs show mean+/−SD. **** p<0.0001, *** p<0.001, ** p<0.01, * p<0.05.

FIGS. 7A-7K show loss of KIF18A causes centrosome fragmentation in CIN cells. FIG. 7A shows representative images of MDA-MB-231 cells treated with either control (top) or KIF18A (bottom) siRNA. Pericentriolar material (γ-tubulin) is numbered to show poles with and without centrioles (centrin-1). Scale bars are 10 microns. FIG. 7B is a graph of the percent of mitotic cells with multipolar spindles from fixed cell images of each indicated cell line treated with either control or KIF18A siRNA. FIG. 7C is a plot of multipolar spindle percentage as a function of fold-change (FC) in cell number for the indicated cell lines following KIF18A KD. R-squared value is 0.84 using a linear regression model. FIG. 7D shows representative images of a MDA-MB-231 cell with a third pole lacking centrin-1. Scale bar is 10 microns. FIG. 7E is a graph of the percent of multipolar MDA-MB-231 cells in mitosis with fragmented pericentriolar material (PCM), as indicated by the presence of γ-tubulin puncta lacking centrin-1 puncta. FIG. 7F is a plot of the intercentriolar distance measurements (in microns) for MDA-MB-231 cells in each indicated category. FIG. 7G shows representative still frames of a live MDA-MB-231 KIF18A KD cell labeled with siR-tubulin. Arrows indicate pole splitting and separation. FIGS. 7H-7I are plots of the percent of live, siR-tubulin labeled MDA-MB-231 cells that (FIG. 7H) enter mitosis with more than two spindle poles or (FIG. 7I) split and separate spindle poles during mitosis. All graphs show mean+/−SD. **** p<0.0001, *** p<0.001, ** p<0.01, * p<0.05. FIGS. 7J and 7K show the centrosome fragmentation in KIF18A KD cells does not require bipolar spindle formation. FIG. 7J is a graph of the percent of monopolar MDA-MB-231 cells with three or more γ-tubulin puncta in control or KIF18A KD cells treated with both monastrol (20 μM) and either DMSO or 20 nM Paclitaxel (Pac). n (number of monopolar mitotic cells/number of independent experiments)=112/4 (control KD+DMSO), 102/4 (KIF18A KD+DMSO), 132/3 (control KD+Pac), and 149/3 (KIF18A KD+Pac). The graph shows mean±SD with individual data points indicated. Data were analyzed via a two-sided Chi-square test, and P values <0.05 are displayed. FIG. 7K is representative images (from three independent experiments) of MDA-MB-231 cells treated with 20 μM monastrol and either DMSO or 20 nM Paclitaxel. DNA (DAPI), microtubules (α-tubulin), and centrosomes (γ-tubulin) are labeled. Scale bar is 5 microns.

FIGS. 8A-8F show spindle checkpoint inhibition rescues mitotic arrest but not multipolar spindle formation caused by KIF18A KD. FIGS. 8A-8B are graphs of the percent of fixed MDA-MB-231 cells (FIG. 8A) in mitosis or (FIG. 8B) with multipolar spindles after the indicated siRNA KD. Results are from three independent experiments. FIGS. 8C-8D are plots of the percent of live, siR-tubulin labeled MDA-MD-231 cells that (FIG. 8C) split poles during mitosis or (FIG. 8D) entered mitosis with more than two spindle poles. Results are from two independent experiments. FIG. 8E is a stacked histogram showing relative frequencies of the duration of time between NEB and pole splitting for siR-tubulin labeled MDA-MB-231 cells following KIF18A KD or KIF18A/MAD2 KD. FIG. 8F shows Western blots depicting the amount of each specified protein remaining after treatment with either a double dose of control siRNA or a combination of KIF18A and MAD2 siRNA. All graphs show mean+/−SD. **** p<0.0001, *** p<0.001, ** p<0.01, * p<0.05.

FIGS. 9A-9C show KIF18A KD-induced centrosome fragmentation is reduced by paclitaxel and nocodazole. FIG. 9A is a graph of the percentage of mitotic cells with multipolar spindles in fixed MDA-MB-231 or MCF10A cells treated with control siRNAs, KIF18A siRNAs, 10 nM paclitaxel, or DMSO. FIG. 9B is a graph of the percentage of fixed MDA-MB-231 and MCF10A cells in mitosis following treatment with control siRNAs, KIF18A siRNAs, 10 nM paclitaxel, or DMSO. All graphs show mean+/−SD. **** p<0.0001, *** p<0.001, ** p<0.01, * p<0.05. FIG. 9C is a graph of the percent of MDA-MB-231 cells with multipolar spindles in control or KIF18A KD cells treated with either DMSO, 20 nM Paclitaxel (Pac), or 5 μM Nocodazole (Noc) for 3 h. n=151 (control KD+DMSO), 263, (KIF18A KD+DMSO), 189 (control KD+pac), 218 (KIF18A KD+pac), 155 (control KD+noc), and 158 (KIF18A KD+noc) cells from three independent experiments. Data were analyzed via a two-sided Chi-square test.

FIGS. 10A-10C show increasing MCAK activity synergistically enhances KIF18A KD defects in CIN cells. FIG. 10A is a plot of fold change in cell density after 96 hours in MDA-MB-231 cells treated with the specified siRNAs and either 500 nM UMK57 or DMSO. FIGS. 10B-10C are graphs of the percent of (FIG. 10B) total mitotic cells and (FIG. 10C) mitotic cells with multipolar spindles in fixed populations after the indicated treatment. At least 60 fields from three independent experiments were analyzed per condition. All graphs show mean+/−SD. **** p<0.0001, *** p<0.001, ** p<0.01, * p<0.05.

FIGS. 11A-11E show KIF18A KD synergizes with the MCAK agonist UMK57 to reduce proliferation and increase spindle pole fragmentation in CIN cells. FIG. 11A show representative images of cell density 96 hours after the start of high-contrast brightfield imaging. Cells were treated with either control or KIF18A siRNA in combination with 500 nM UMK57 or DMSO. Scale bar is 100 microns. FIGS. 11B-11C are graphs of percent of live, siR-tubulin labeled MDA-MB-231 cells that (FIG. 11B) split poles or (FIG. 11C) entered mitosis with more than two spindle poles after the indicated treatments. FIGS. 11D-11E show representative immunofluorescence images of mitotic MDA-MB-231 cells treated with either (FIG. 11D) control or (FIG. 11E) KIF18A siRNA in combination with either 500 nM UMK57 or DMSO. Scale bars are 10 microns.

FIGS. 12A-12C show the proliferation and multipolar spindle defects caused by KIF18A KD are sensitive to changes in KIF2C/MCAK activity. FIG. 12A is a graph of the percent of MDA-MB-231 cells with multipolar spindles following transfection with the indicated siRNAs and mCh-full-length-MCAK (FL MCAK) or mCh-CPB-MCAK (CPB MCAK), which localizes to centromeres via the CENP-B DNA-binding domain. n=102 (control KD+FL MCAK), 202 (KIF18A KD+FL MCAK), 113 (control KD+CPB MCAK), and 187 cells (KIF18A KD+CPB MCAK) from two independent experiments. FIGS. 12B and 12C are graphs of the percent of live, siR-tubulin labeled MDA-MB-231 cells that (FIG. 12B) split poles or (FIG. 12C) entered mitosis with more than two spindle poles after treatment with the indicated siRNAs. n (number of cells/number of independent experiments)=100/2 (control KD), 106/2 (KIF18A KD), 111/2 (KIF18A+MCAK KD), and 51/1 (MCAK KD). All graphs show mean and individual data points.

### DETAILED DESCRIPTION

A first aspect of the present disclosure is directed to a method of inhibiting proliferation of chromosome instable cancer cells. This method involves administering, to a population of cancer cells comprising chromosome instable cancer cells, an inhibitor of Kinesin Family Member 18A (KIF18A) at a dosage effective to inhibit proliferation of said chromosome instable cancer cells.

A related aspect of the present disclosure is directed to a method of treating cancer in a subject, this method involves administering to a subject having cancer, wherein said cancer is characterized by chromosomal instability, an inhibitor of KIF18A at a dosage effective to treat the cancer in the subject.

Suitable cancer cells and/or cancers that can be treated in accordance with the methods described herein include cancers characterized by chromosome instable cancer cells. Chromosomal instability (CIN) is characterized by frequent missegregation of chromosomes during mitosis. CIN is a hallmark of tumor cells caused by changes in the dynamics and control of microtubules that comprise the mitotic spindle. As referred to herein, cancers characterized by chromosome instability comprise cancer cells having an altered number of chromosomes, e.g., aneuploidy or polyploidy, cancer cells having abnormal microtubule dynamics, cancer cells having abnormal chromosomal structure, e.g., chromosome deletions, translocations, additions, or any combination of these characteristics.

As described herein altered microtubule dynamics in mitotic CIN cells renders these cells dependent on KIF18A to reduce kinetochore microtubule turnover, which is required to maintain spindle bipolarity and promote mitotic progression. However, KIF18A is not required for mitosis or proliferation of near diploid cells. Thus, KIF18A inhibition is an effective target to specifically inhibit the growth of CIN tumor cells, while inducing relatively low toxicity in somatic, diploid cells.

Numerous cancer types and cells exhibit CIN, including, without limitation, breast cancer cells, bladder cancer cells, colorectal cancer cells, prostate cancer cells, cervical cancer cells, endometrial cancer cells, lung cancer cells, liver cancer cells, high hyperdiploid acute lymphoblastic leukemia cells, ovarian cancer cells, and glioblastoma cells. See Vargas-Rondon et al., “The Role of Chromosomal Instability in Cancer and Therapeutic Responses,” Cancers 10:4 (2018), which is hereby incorporated by reference in its entirety.

Breast cancer and breast cancer cells that can be treated in accordance with the methods described herein include, without limitation, those forms exhibiting CIN. In particular, breast cancers characterized by cancer cells having an altered number of chromosomes, i.e., aneuploid or polyploid cancer cells. Suitable breast cancers also include those exhibiting altered chromosome structure. For example, estrogen receptor positive breast cancer, basal-like tumors, and HER2-related tumors that exhibit gains and losses of whole chromosome arms, e.g., gain of 1q, 16p and loss of 16q are suitable for treatment with a KIF18A inhibitor alone or together with an agent that promotes microtubule turnover as described herein. Luminal B and HER2-related tumors exhibiting DNA amplifications, for example amplification at 8p12 (FGFR1), 8q24 (MYC), 11q13 (CCND1), 12q15 (MDM2), 17q12 (HER2), and 20q13 (ZNF217), and triple-negative and basal-like breast cancer forms exhibiting complex patterns of many gains and losses of chromosomal arms can also be treated in accordance with the methods described herein.

Prostate cancer and prostate cancer cells that can be treated in accordance with the methods described herein include, without limitation, those forms exhibiting CIN. In particular, prostate cancers characterized by cancer cells having an altered number of chromosomes, i.e., aneuploid or polyploidy cancer cells. Suitable prostate cancers also include those exhibiting altered chromosome structure. For example, prostate cancer having chromosomal gains in chromosomes 8, 7 and Y are suitable for treatment with a KIF18A inhibitor alone or together with an agent that promotes microtubule turnover as described herein. Metastatic prostate cancer exhibiting CIN that can be treated with the methods disclosed herein include, without limitation, those forms having chromosomal losses in 8p23, 10q, 13q and 16q, and gains in 8q and Xq.

Colorectal cancer and colorectal cancer cells that can be treated in accordance with the methods described herein include, without limitation, stages 1-4 forms exhibiting CIN. In particular, colorectal cancers characterized by cancer cells having an altered number of chromosomes, i.e., aneuploid or polyploidy cancer cells. Suitable colorectal cancers also include those exhibiting altered chromosome structures. For example, colorectal forms exhibiting losses at 16p13, 19q13, and 18q21, or imbalances on chromosomes 1p, 5q, 8p, 15q, and 18q can all be treated in accordance with the methods described herein. Colorectal polyps exhibiting losses of chromosomes 17p, 19q and 22q and the gains of chromosomes 7 and 13 are also suitable for treatment.

Cervical cancer and cervical cancer cells that can be treated in accordance with the methods described herein include, without limitation, those forms exhibiting CIN. These forms include, without limitation, those exhibiting structural and numerical chromosome 1 alterations, and monosomies and polysomies of chromosomes 1, 3, and X.

Endometrial cancer and endometrial cancer cells that can be treated in accordance with the methods described herein include, without limitation, those forms exhibiting CIN. In particular, endometrial cancers characterized by cancer cells having an altered number of chromosomes, i.e., aneuploid or polyploid cancer cells. Suitable endometrial cancers also include those exhibiting altered chromosome structure. For example, endometrial cancers exhibiting gains of chromosomes 1 and 10 are suitable for treatment with a KIF18A inhibitor alone or together with an agent that promotes microtubule turnover as described herein.

Bladder cancer and bladder cancer cells that can be treated in accordance with the methods described herein include, without limitation, those forms exhibiting CIN. In particular, bladder cancers characterized by cancer cells having an altered number of chromosomes, i.e., aneuploid or polyploid cancer cells. Suitable bladder cancers also include those exhibiting altered chromosome structure. Numerous, nonrandom chromosomal deletions detected in bladder cancer include deletions of 3p, 8p, 9p, 11p, 11q and Y, and gains of 1q, 8q, 17q and 20q have also been found. All of these forms of bladder cancer are suitable for treatment with a KIF18A inhibitor alone or together with an agent that promotes microtubule turnover as described herein.

Multiple myeloma that can be treated in accordance with the methods described herein include, without limitation, those forms exhibiting CIN. These form include, without limitation, those exhibiting abnormalities such as t(4:14) and the deletion of the short art of chromosome 17.

High Hyperdiploid Acute Lymphoblastic Leukemia (HeH ALL) that can be treated in accordance with the methods described herein include, without limitation, those forms exhibiting CIN. For example, HeH ALL forms characterized by nonrandom gains of chromosomes X, 4, 6, 10, 14, 17, 18, and 21 are suitable for treatment with a KIF18A inhibitor alone or together with an agent that promotes microtubule turnover as described herein.

Lung cancer and lung cancer cells that can be treated in accordance with the methods described herein include, without limitation, those forms exhibiting CIN. In particular, lung cancers characterized by cancer cells having an altered number of chromosomes, i.e., aneuploid or polyploid cancer cells. Suitable lung cancers also include those exhibiting altered chromosome structures (e.g., deletions, translocations, and isochromosomes). In particular, non-small cell lung cancers (NSCLC) having chromosome gains in any one of chromosomes 5p, 8q, 17q, and 19q, and chromosome losses in any one of chromosomes 1p, 4q, 5q, 6q, 8p, 9p, 13q and 17p. Also suitable for treatment in accordance with the methods described herein are small cell lung carcinomas (SCLC) exhibiting chromosomal gains in chromosomes 3q, 5p, 8p, and Xq, or chromosomal losses in chromosomes 5q, 13q and 17p.

Liver cancer and liver cancer cells that can be treated in accordance with the methods described herein include, without limitation, those forms exhibiting CIN. In particular, liver cancers characterized by cancer cells having an altered number of chromosomes, i.e., aneuploid or polyploid cancer cells. Suitable liver cancers also include those exhibiting altered chromosome structures. There are a number of genes frequently mutated in liver cancer that are associated with CIN. These liver cancers, which are suitable for treatment in accordance with the methods described herein include, without limitation, liver cancers characterized by a loss of function p53 gene mutation, a gain of function beta-catenin gene mutation, mutation in WWP1 (WW domain-containing Protein 1/NEDD4-like E3 ubiquitin protein ligase), mutation in RPS6KA3, and loss of function mutation in MLL2/KMT2D (see Rao et al., “Frequently Mutated Genes/Pathways and Genomic Instability as Prevention Targets in Liver Cancer,” Carcinogenesis 38(10): 2-11 (2017), which is hereby incorporated by reference in its entirety).

Suitable “subjects” for treatment in accordance with the methods described herein include any subject, e.g., animal or human, having a chromosome instable form of cancer. Preferably, the subject is a mammal. Exemplary mammalian subjects include, without limitation, humans, non-human primates, dogs, cats, rodents (e.g., mouse, rat, guinea pig), horses, cattle and cows, sheep, and pigs. In some embodiments, the subject is a human subject.

In accordance with this and all aspects of the disclosure an inhibitor of KIF18A is any agent that inhibits the expression and/or activity of KIF18A. Suitable KIF18A inhibitors include protein inhibitors, e.g., an anti-KIF18A antibody or binding fragment thereof, a nucleic acid inhibitor, e.g., siRNA or antisense oligonucleotide, or a small molecule inhibitor.

Kinesins are a family of proteins that coordinate the process of chromosome segregation during cell division. KIF18A is a member of this family that plays a central role in aligning chromosomes at the spindle equator, and exhibits both motility and depolymerase activity. See Stumpff and Wordeman, “Chromosome Congression: The Kinesin-8-Step Path to Alignment,” Curr. Biol. 17(9): R326-328 (2007), which is hereby incorporated by reference in its entirety. The nucleotide and amino acid sequences of human KIF18A are well known in the art. The nucleotide sequence encoding KIF18A is provided below as SEQ ID NO: 1 (NCBI Ref. Seq. No. NM_31217.3).

| (SEQ ID NO: 1) | |
|---|---|
| 1 | aatgaaacga agcgctgagg aaagtggctt gggtttgaat attgtggttg agtctgaagc |
| 61 | gctgggaggc ggacattaaa gtgaagtggt tgcggtaacc tggcctgggc ctgaagtgag |
| 121 | tgagaggcac atgaagagaa gtattcaagt atttatacag ataggaatca agataatcaa |
| 181 | caatgtctgt cactgaggaa gacctgtgcc accatatgaa agtagtagtt cgtgtacgtc |
| 241 | cggaaaacac taaagaaaaa gcagctggat ttcataaagt ggttcatgtt gtggataaac |
| 301 | atatcctagt ttttgatccc aaacaagaag aagtcagttt tttccatgga aagaaaacta |
| 361 | caaatcaaaa tgttataaag aaacaaaata aggatcttaa atttgtattt gatgctgttt |
| 421 | ttgatgaaac gtcaactcag tcagaagttt ttgaacacac tactaagcca attcttcgta |
| 481 | gttttttgaa tggatataat tgcacagtac ttgcctatgg tgccactggt gctgggaaga |
| 541 | cccacactat gctaggatca gctgatgaac ctggagtgat gtatctaaca atgttacacc |
| 601 | tttacaaatg catggatgag attaaagaag agaaaatatg tagtactgca gtttcatatc |
| 661 | tggaggtata taatgaacag attcgtgatc tcttagtaaa ttcagggcca cttgctgtcc |
| 721 | gggaagatac ccaaaaaggg gtggtcgttc atggacttac tttacaccag cccaaatcct |
| 781 | cagaagaaat tttacattta ttggataatg gaaacaaaaa caggacacaa catcccactg |
| 841 | atatgaatgc cacatcttct cgttctcatg ctgttttcca aatttacttg cgacaacaag |
| 901 | acaaaacagc aagtatcaat caaaatgtcc gtattgccaa gatgtcactc attgacctgg |
| 961 | caggatctga gcgagcaagt acttccggtg ctaaggggac ccgatttgta gaaggcacaa |
| 1021 | atattaatag atcactttta gctcttggga atgtcatcaa tgccttagca gattcaaaga |
| 1081 | gaaagaatca gcatatccct tacagaaata gtaagcttac tcgcttgtta aaggattctc |
| 1141 | ttggaggaaa ctgtcaaact ataatgatag ctgctgttag tccttcctct gtattctacg |
| 1201 | atgacacata taacactctt aagtatgcta accgggcaaa ggacattaaa tcttctttga |
| 1261 | agagcaatgt tcttaatgtc aataatcata taactcaata tgtaaagatc tgtaatgagc |
| 1321 | agaaggcaga gattttattg ttaaaagaaa aactaaaagc ctatgaagaa cagaaagcct |
| 1381 | tcactaatga aaatgaccaa gcaaagttaa tgatttcaaa ccctcaggaa aaagaaatcg |
| 1441 | aaaggtttca agaaatcctg aactgcttgt tccagaatcg agaagaaatt agacaagaat |
| 1501 | atctgaagtt ggaaatgtta cttaaagaaa atgaacttaa atcattctac caacaacagt |
| 1561 | gccataaaca aatagaaatg atgtgttctg aagacaaagt agaaaaggcc actggaaaac |
| 1621 | gagatcatag acttgcaatg ttgaaaactc gtcgctccta cctggagaaa aggagggagg |
| 1681 | aggaattgaa gcaatttgat gagaatacta attggctcca tcgtgtcgaa aaagaaatgg |
| 1741 | gactcttaag tcaaaacggt catattccaa aggaactcaa gaaagatctt cattgtcacc |
| 1801 | atttgcacct ccagaacaaa gatttgaaag cacaaattag acatatgatg gatctagctt |
| 1861 | gtcttcagga acagcaacac aggcagactg aagcagtatt gaatgcttta cttccaaccc |
| 1921 | taagaaaaca atattgcaca ttaaaagaag ccggcctgtc aaatgctgct tttgaatctg |
| 1981 | acttcaaaga gatcgaacat ttggtagaga ggaaaaaagt ggtagtttgg gctgaccaaa |
| 2041 | ctgccgaaca accaaagcaa aacgatctac cagggatttc tgttcttatg acctttccac |
| 2101 | aacttggacc agttcagcct attccttgtt gctcatcttc aggtggaact aatctggtta |
| 2161 | agattcctac agaaaaaaga actcggagaa aactaatgcc atctcccttg aaaggacagc |
| 2221 | atactctaaa gtctccacca tctcaaagtg tgcagctcaa tgattctctt agcaaagaac |
| 2281 | ttcagcctat tgtatataca ccagaagact gtagaaaagc ttttcaaaat ccgtctacag |
| 2341 | taaccttaat gaaaccatca tcatttacta caagttttca ggctatcagc tcaaacataa |
| 2401 | acagtgataa ttgtctgaaa atgttgtgtg aagtagctat ccctcataat agaagaaaag |
| 2461 | aatgtggaca ggaggacttg gactctacat ttactatatg tgaagacatc aagagctcga |
| 2521 | agtgtaaatt acccgaacaa gaatcactac caaatgataa caaagacatt ttacaacggc |
| 2581 | ttgatccttc ttcattctca actaagcatt ctatgcctgt accaagcatg gtgccatcct |
| 2641 | acatggcaat gactactgct gccaaaagga aacggaaatt aacaagttct acatcaaaca |
| 2701 | gttcgttaac tgcagacgta aattctggat ttgccaaacg tgttcgacaa gataattcaa |
| 2761 | gtgagaagca cttacaagaa aacaaaccaa caatggaaca taaaagaaac atctgtaaaa |
| 2821 | taaatccaag catggttaga aaatttggaa gaaatatttc aaaaggaaat ctaagataaa |
| 2881 | tcacttcaaa accaagcaaa atgaagttga tcaaatctgc ttttcaaagt ttatcaatac |
| 2941 | cctttcaaaa atatatttaa aatctttgaa agaagaccca tcttaaagct aagtttaccc |
| 3001 | aagtactttc agcaagcaga aaaatgaaac tctttgtttt cttcttttgt gttctaaaaa |
| 3061 | aataaaattt caaaagaaaa ggttgtcttt taagtttttt aaatatttgt tgccttttaa |
| 3121 | aatccctgag tgtaagttac catggtggca gcttagtttt actatgccac aacaagttga |
| 3181 | ctaggacatt ttagtaaatg gtagtgagtt aaattatctt tattattttt taaaaataag |
| 3241 | aatttagaag tggtaaaatt atggcccaag atgtatttgg ttctctatta tgttttgata |
| 3301 | cattatttta atcatatata tgactttcct tttcaaaaat actttaatgt acaagtgtaa |
| 3361 | atatatgtgc ccataaaatc attgtaaata ttatttagtc atcacaaata aaatattgtc |
| 3421 | ccttgctact tgatatatta aagatgtaga ttttaaagtg ttt |

The amino acid sequence of KIF18A is provided below as SEQ ID NO: 2 (UniProtKB Ref. NO. Q8N177):

(SEQ ID NO: 2)
MSVTEEDLCHHMKVVVRVRPENTKEKAAGFHKVVHVVDKHILVFDPKQ
EEVSFFHGKKTTNQNVIKKQNKDLKFVFDAVFDETSTQSEVFEHTTKP
ILRSFLNGYNCTVLAYGATGAGKTHTMLGSADEPGVMYLTMLHLYKCM
DEIKEEKICSTAVSYLEVYNEQIRDLLVNSGPLAVREDTQKGVVVHGL
TLHQPKSSEEILHLLDNGNKNRTQHPTDMNATSSRSHAVFQIYLRQQD
KTASINQNVRIAKMSLIDLAGSERASTSGAKGTRFVEGTNINRSLLAL
GNVINALADSKRKNQHIPYRNSKLTRLLKDSLGGNCQTIMIAAVSPSS
VFYDDTYNTLKYANRAKDIKSSLKSNVLNVNNHITQYVKICNEQKAEI
LLLKEKLKAYEEQKAFTNENDQAKLMISNPQEKEIERFQEILNCLFQN
REEIRQEYLKLEMLLKENELKSFYQQQCHKQIEMMCSEDKVEKATGKR
DHRLAMLKTRRSYLEKRREEELKQFDENTNWLHRVEKEMGLLSQNGHI
PKELKKDLHCHHLHLQNKDLKAQIRHMMDLACLQEQQHRQTEAVLNAL
LPTLRKQYCTLKEAGLSNAAFESDFKEIEHLVERKKVVVWADQTAEQP
KQNDLPGISVLMTFPQLGPVQPIPCCSSSGGTNLVKIPTEKRTRRKLM
PSPLKGQHTLKSPPSQSVQLNDSLSKELQPIVYTPEDCRKAFQNPSTV
TLMKPSSFTTSFQAISSNINSDNCLKMLCEVAIPHNRRKECGQEDLDS
TFTICEDIKSSKCKLPEQESLPNDNKDILQRLDPSSFSTKHSMPVPSM
VPSYMAMTTAAKRKRKLTSSTSNSSLTADVNSGFAKRVRQDNSSEKHL
QENKPTMEHKRNICKINPSMVRKFGRNISKGNLR.

Small molecule inhibitors of KIF18A are known in the art and suitable for use in the methods disclosed herein. See e.g., Catarinella et al., “BTB-1: A Small Molecule Inhibitor of the Mitotic Motor Protein Kif18A,” Angew. Chem Int. Ed. 48:9072-76 (2009) and Braun et al., “Synthesis and Biological Evaluation of Optimized Inhibitors of the Mitotic Kinesin Kif18A,” ACS Chem. Biol. 10:554-560 (2015), which are hereby incorporated by reference in their entirety. In some embodiments, the KIF18A inhibitor comprises the compound of Formula I

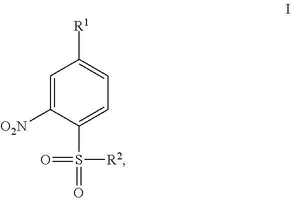

or a derivative thereof,
wherein

R¹ is selected from NO₂, F, Cl, CF₃, and H; and

R² is selected from phenyl or 2-thiophene.

In some embodiments the KIF18A inhibitor of Formula I, R¹ is Cl and R² is phenyl. In some embodiments, R¹ is NO₂, and R² is phenyl. In some embodiments R¹ is F and R² is phenyl. In some embodiments R¹ is CF₃ and R² is phenyl. In some embodiments R¹ is Cl and R² is 2-thiophene. In some embodiments R¹ is H and R² is phenyl.

Further examples of KIF18A inhibitors suitable for use in the methods of the present disclosure are disclosed in Sabnis, “Novel KIF18A Inhibitors for Treating Cancer,” ACS Med. Chem. Lett. 11:2079-2080 (2020) and in PCT Application Publication No. WO 2020132651 to Tamayo et al., which are hereby incorporated by reference in their entirety. In some embodiments, the KIF18A inhibitor comprises the compound of Formula II

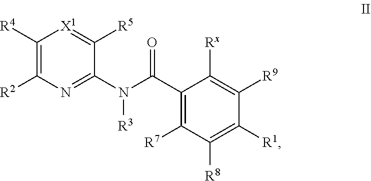

or a derivative thereof,
wherein

X¹ is N or —CR⁶;

R¹ is —Z—R¹² wherein Z is selected from —C₀₋₄alkyl-, —NR¹¹—, —NR¹¹SO₂—C₀₋₄alkyl-, —SO₂NR¹¹—C₀₋₄alkyl-, —NR¹¹SO₂NR¹¹—, —NR¹¹SO₂NR¹¹—C(═O)—O—, —C₀₋₄alkyl-S(═O)(═NH)—, C₀₋₄alkyl-NR¹¹—S(═O)(═NH), —C₀₋₄alkyl-S—, —C₀₋₄alkyl-S(═O)—, —C₀₋₄alkyl-SO₂—, C₀₋₄alkyl-O—, —P—, —P(═O), —P(═O)₂, —(C═O)—, —(C═O)NR¹¹—, —C═N(OH)—, or —NR¹¹(C═O); or the group —Z—R¹² is —N═S(═O)—(R¹²)₂, wherein the two R¹² pair can alternatively combine with the sulfur atom attached to each of them to form a saturated or partially-saturated 3-, 4-, 5-, or 6-membered monocyclic ring containing 0, 1, 2 or 3 N atoms and 0, 1, or 2 atoms selected from O and S;

R² is halo or —Y—R¹³, wherein Y is —C₀₋₄alkyl-, —N(C₀₋₁alkyl)-C₀₋₄alkyl-, —C(═O)NR^{a}R^{a}(C₁₋₄alkyl)-, —O—C₀₋₄alkyl-, —S—, —S═O, —S(═O)₂—, —SO₂N(C₀₋₁alkyl)-C₀₋₄alkyl-, —N(C₀₋₁alkyl)-SO₂—C₀₋₄alkyl-, —C₀₋₄alkyl-S(═O)(═NH)—, —(C═O)—, —C₀₋₄alkyl-(C═O)—O—; or the group —Y—R¹³ is —N═S(═O)—(R¹³)₂, wherein the two R¹³ pair can alternatively combine with the sulfur atom attached to each of them to form a saturated or partially-saturated 3-, 4-, 5-, or 6-membered monocyclic ring containing 0, 1, 2 or 3 N atoms and 0, 1, or 2 atoms selected from O and S;

R³ is H, methyl, or ethyl;

R⁴ is H, halo, CN, C₁₋₄alk, or C₁₋₄haloalk;

R⁵ is H, halo, C₁₋₈alk, or C₁₋₄haloalk;

R⁶ is H, halo, CN, C₁₋₈alk, C₁₋₄haloalk, —O—C₀₋₆alkyl-, or R^{6a};

R⁷, R⁸, and R⁹ are independently selected from H, halo, C₁₋₈alkyl, or C₁₋₄haloalkyl;

R^{x} is selected from the group consisting of

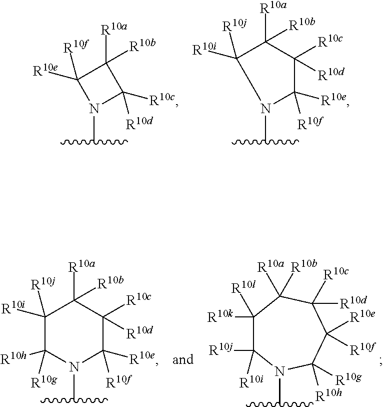

R^{10a-10j} are independently selected from H, halo, R^{10k}, or R^{10L}; or alternatively, each of R^{10a} and R^{10b} pair, R^{10c} and R^{10d} pair, R^{10e} and R^{10f} pair, R^{10g} and R^{10h} pair, or R¹⁰ⁱ and R^{10j} pair, independently, can combine with the carbon atom attached to each of them to form a saturated or partially-saturated 3-, 4-, 5-, 6-membered monocyclic ring spiro to the R^{x} ring; wherein said 3-, 4-, 5-, 6-membered monocyclic ring contains 0, 1, 2 or 3 N atoms and 0, 1, or 2 atoms selected from 0 and S, and further wherein said 3-, 4-, 5-, 6-membered monocyclic ring is substituted by 0, 1, 2 or 3 group(s) selected from F, Cl, Br, C₁₋₆alkyl, C₁₋₄haloalkyl, —OR^{a}, —OC₁₋₄haloalkyl, CN, —NR^{a}R^{a}, or oxo;

R¹¹ is H or C₁₋₈alkyl;

R¹² is H, R^{12a}, or R^{12b}.

R¹³ is R^{13a} or R^{13b};

R^{6a}, R^{10k}, R^{12a}, and R^{13a} are independently selected at each occurrence from the group consisting of a saturated, partially-saturated or unsaturated 3-, 4-, 5-, 6-, or 7-membered monocyclic or 4-, 5-, 6-, 7-, 8-, 9-, 10-, 11-, or 12-membered bicyclic ring containing 0, 1, 2 or 3 N atoms and 0, 1, or 2 atoms selected from O and S, which is substituted by 0, 1, 2 or 3 group(s) selected from F, Cl, Br, C₁₋₆alkyl, C₁₋₄haloalkyl, —OR^{a}, —OC₁₋₄haloalkyl, CN, —C(═O)R^{b}, —C(═O)OR^{a}, —C(═O)NR^{a}R^{a}, —C(═NR^{a})NR^{a}R^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a}, —NR^{a}C₂₋₆alkOR^{a}, —C₁-6alkNR^{a}R^{a}, —C₁₋₆alkOR^{a}, —C₁₋₆alkN(R^{a})C(═O)R^{b}, —C₁₋₆alkOC(═O)R^{b}, —C₁₋₆alkC(═O)NR^{a}R^{a}, —C₁₋₆alkC(═O)OR^{a}, R¹⁴, and oxo;

R^{10l}, R^{12b}, and R^{13b} are independently selected at each occurrence from the group consisting of C₁₋₆alkyl substituted by 0, 1, 2, 3, 4, or 5 group(s) selected from F, Cl, Br, —C(═O)OR^{a}, —OR^{a}, —C₁₋₂haloalk, —OC₁₋₄haloalk, CN, NH₂, NH(CH₃), or N(CH₃)₂;

R¹⁴ is independently, at each instance, selected from the group consisting of a saturated, partially-saturated or unsaturated 3-, 4-, 5-, 6-, or 7-membered monocyclic or 4-, 5-, 6-, 7-, 8-, 9-, 10-, 11-, or 12-membered bicyclic ring containing 0, 1, 2 or 3 N atoms and 0, 1, or 2 atoms selected from O and S, which is substituted by 0, 1, 2 or 3 group(s) selected from F, Cl, Br, C₁₋₆alk, C₁₋₄haloalk, —OR^{a}, —OC₁₋₄haloalk, CN, —C(═O)R^{b}, —C(═O)OR^{a}, —C(═O)NR^{a}R^{a}, —C(═NR^{a})NR^{a}R^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a}, —NR^{a}C₂₋₆alkOR^{a}, —C₁₋₆alkNR^{a}R^{a}, —C₁₋₆alkOR^{a}, —C₁₋₆alkN(R^{a})C(═O)R^{b}, —C₁₋₆alkOC(═O)R^{b}, —C₁₋₆alkC(═O)NR^{a}R^{a}, —C₁₋₆alkC(═O)OR^{a}, and oxo;

R^{a} is independently selected at each occurrence from H or R^{b}; and

R^{b} is independently, at each instance, C₁₋₆alkyl, phenyl, or benzyl, wherein the C₁₋₆alkyl may be substituted by 1, 2 or 3 substituents selected from halo, —OH, —OC₁₋₄alkyl, —NH₂, —NHC₁₋₄alkyl, —OC(═O)C₁₋₄alkyl, or —N(C₁₋₄alkyl)C₁₋₄alkyl; and the phenyl or benzyl may be substituted by 1, 2 or 3 substituents selected from halo, C₁₋₄alkyl, C₃haloalkyl, —OH, —OC₄alkyl, —NH₂, —NHC₁₋₄alkyl, —OC(═O)C₁₋₄alkyl, or —N(C₁₋₄alkyl)C₁₋₄alkyl.

Exemplary KIF18A compounds of formula II that may be used in method of the present disclosure include, but are not limited to, the compounds in Table 1 below.

TABLE 1
Exemplary KIF18A inhibitors of formula II

| Chemical Structure | Name |
|---|---|
| 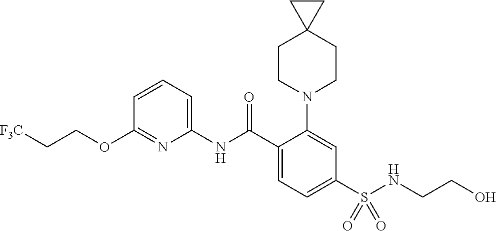 [SMILES: O=C(Nc1cccc(OCCC(F)(F)F)n1)c1ccc(S(=O)(=O)NCCO)cc1N1CCC2(CC1)CC2] | 4-(N-(2-Hydroxyethyl)sulfamoyl)-2-(6- azaspiro[2.5]octan-6-yl)-N-(6-(3,3,3- trifluoropropoxy)pyridin-2- yl)benzamide |
| 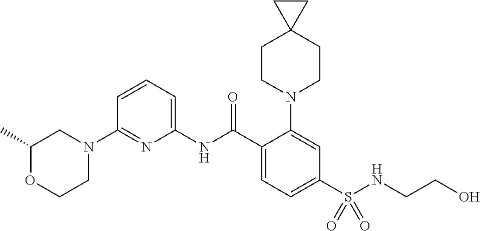 [SMILES: C[C@@H]1CN(c2cccc(NC(=O)c3ccc(S(=O)(=O)NCCO)cc3N3CCC4(CC3)CC4)n2)CCO1] | (R)-4-(N-(2-Hydroxyethyl)sulfamoyl)-N-(6- (2-methylmorpholino)pyridin-2-yl)-2-(6- azaspiro[2.5]octan-6-yl)benzamide |
| 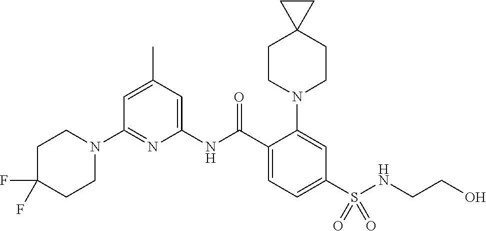 [SMILES: Cc1cc(NC(=O)c2ccc(S(=O)(=O)NCCO)cc2N2CCC3(CC2)CC3)nc(N2CCC(F)(F)CC2)c1] | N-(6-(4,4-Difluoropiperidin-1-yl)-4- methylpyridin-2-yl)-4-(N-(2- hydroxyethyl)sulfamoyl)-2-(6- azaspiro[2.5]octan-6-yl)benzamide |
| 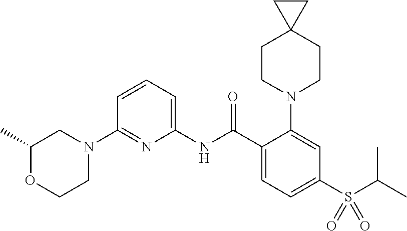 [SMILES: CC(C)S(=O)(=O)c1ccc(C(=O)Nc2cccc(N3CCO[C@H](C)C3)n2)c(N2CCC3(CC2)CC3)c1] | (R)-4-(Isopropylsulfonyl)-N-(6-(2- methylmorpholino)pyridin-2-yl)-2-(6- azaspiro[2.5]octan-6-yl)benzamide |
| 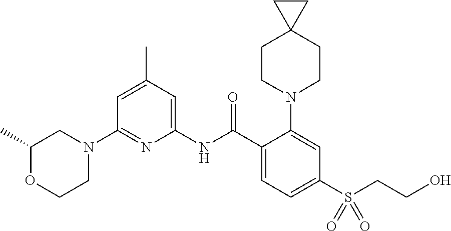 [SMILES: Cc1cc(NC(=O)c2ccc(S(=O)(=O)CCO)cc2N2CCC3(CC2)CC3)nc(N2CCO[C@H](C)C2)c1] | (R)-4-((2-Hydroxyethyl)sulfonyl)-N-(4- methyl-6-(2-methylmorpholino)pyridin-2- yl)-2-(6-azaspiro[2.5]octan-6-yl)benzamide |
| 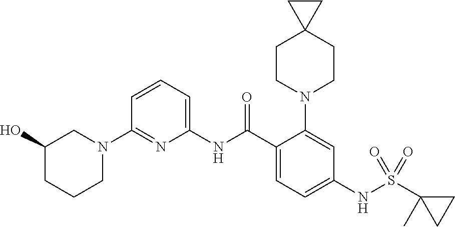 [SMILES: CC1(S(=O)(=O)Nc2ccc(C(=O)Nc3cccc(N4CCC[C@@H](O)C4)n3)c(N3CCC4(CC3)CC4)c2)CC1] | (R)-N-(6-(3-Hydroxypiperidin-1- yl)pyridin-2-yl)-4-((1- methylcyclopropane)-1-sulfonamido)-2- (6-azaspiro[2.5]octan-6-yl)benzamide |
| 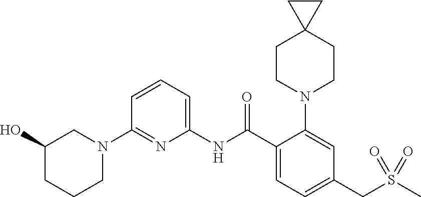 [SMILES: CS(=O)(=O)Cc1ccc(C(=O)Nc2cccc(N3CCC[C@@H](O)C3)n2)c(N2CCC3(CC2)CC3)c1] | (R)-N-(6-(3-hydroxypiperidin-1- yl)pyridin-2-yl)-4- ((methylsulfonyl)methyl)-2-(6- azaspiro[2.5]octan-6-yl)benzamide |
| 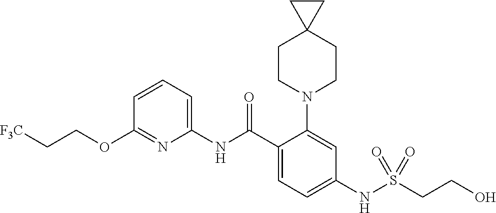 [SMILES: O=C(Nc1cccc(OCCC(F)(F)F)n1)c1ccc(NS(=O)(=O)CCO)cc1N1CCC2(CC1)CC2] | 4-((2-Hydroxyethyl)sulfonamido)-2-(6- azaspiro[2.5]octan-6-yl)-N-(6-(3,3,3- trifluoropropoxy)pyridin-2- yl)benzamide |
| 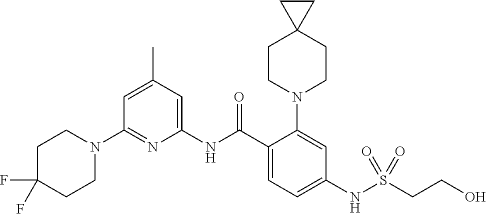 [SMILES: Cc1cc(NC(=O)c2ccc(NS(=O)(=O)CCO)cc2N2CCC3(CC2)CC3)nc(N2CCC(F)(F)CC2)c1] | N-(6-(4,4-Difluoropiperidin-1-yl)-4- methylpyridin-2-yl)-4-((2- hydroxyethyl)sulfonamido)-2-(6- azaspiro[2.5]octan-6-yl)benzamide |
| 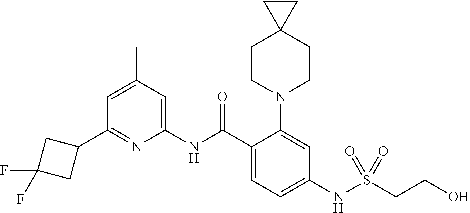 [SMILES: Cc1cc(NC(=O)c2ccc(NS(=O)(=O)CCO)cc2N2CCC3(CC2)CC3)nc(C2CC(F)(F)C2)c1] | N-(6-(3,3-Difluorocyclobutyl)-4- methylpyridin-2-yl)-4-((2- hydroxyethyl)sulfonamido)-2-(6- azaspiro[2.5]octan-6-yl)benzamide |
| 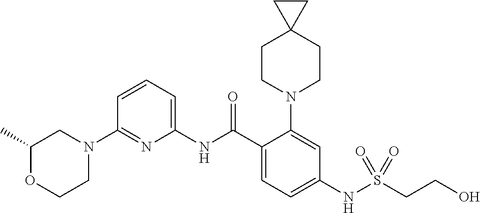 [SMILES: C[C@@H]1CN(c2cccc(NC(=O)c3ccc(NS(=O)(=O)CCO)cc3N3CCC4(CC3)CC4)n2)CCO1] | (R)-4-((2-Hydroxyethyl)sulfonamido)- N-(6-(2-methylmorpholino)pyridin-2- yl)-2-(6-azaspiro[2.5]octan-6- yl)benzamide |
| 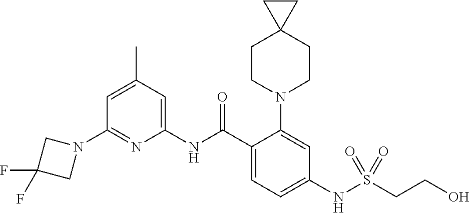 [SMILES: Cc1cc(NC(=O)c2ccc(NS(=O)(=O)CCO)cc2N2CCC3(CC2)CC3)nc(N2CC(F)(F)C2)c1] | N-(6-(3,3-Difluoroazetidin-1-yl)-4- methylpyridin-2-yl)-4-((2- hydroxyethyl)sulfonamido)-2-(6- azaspiro[2.5]octan-6-yl)benzamide |
| 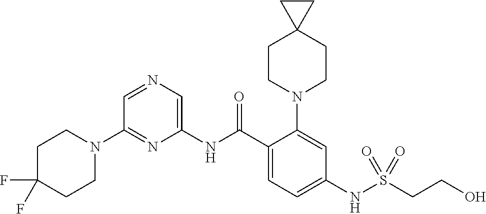 [SMILES: O=C(Nc1cncc(N2CCC(F)(F)CC2)n1)c1ccc(NS(=O)(=O)CCO)cc1N1CCC2(CC1)CC2] | N-(6-(4,4-Difluoropiperidin-1- yl)pyrazin-2-yl)-4-((2- hydroxyethyl)sulfonamido)-2-(6- azaspiro[2.5]octan-6-yl)benzamide |
| 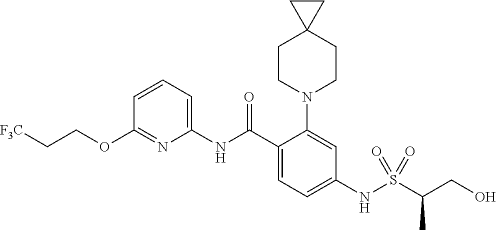 [SMILES: C[C@H](CO)S(=O)(=O)Nc1ccc(C(=O)Nc2cccc(OCCC(F)(F)F)n2)c(N2CCC3(CC2)CC3)c1] | (R)-4-((2-Hydroxy-1- methylethyl)sulfonamido)-2-(6- azaspiro[2.5]octan-6-yl)-N-(6-(3,3,3- trifluoropropoxy)pyridin-2- yl)benzamide |
| 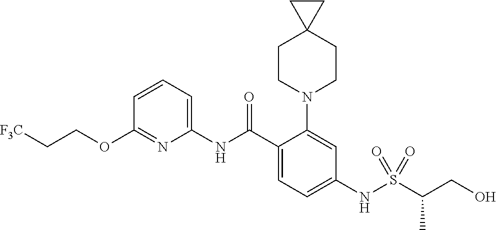 [SMILES: C[C@@H](CO)S(=O)(=O)Nc1ccc(C(=O)Nc2cccc(OCCC(F)(F)F)n2)c(N2CCC3(CC2)CC3)c1] | (S)-4-((2-Hydroxy-1- methylethyl)sulfonamido)-2-(6- azaspiro[2.5]octan-6-yl)-N-(6-(3,3,3- trifluoropropoxy)pyridin-2- yl)benzamide |
| 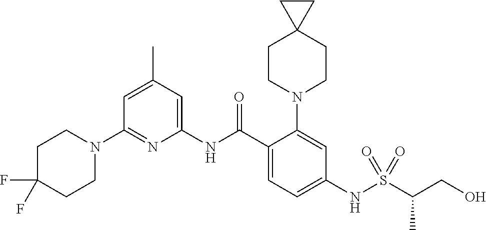 [SMILES: Cc1cc(NC(=O)c2ccc(NS(=O)(=O)[C@@H](C)CO)cc2N2CCC3(CC2)CC3)nc(N2CCC(F)(F)CC2)c1] | (S)-N-(6-(4,4-Difluoropiperidin-1-yl)-4- methylpyridin-2-yl)-4-((2-hydroxy-1- methylethyl)sulfonamido)-2-(6- azaspiro[2.5]octan-6-yl)benzamide |
| 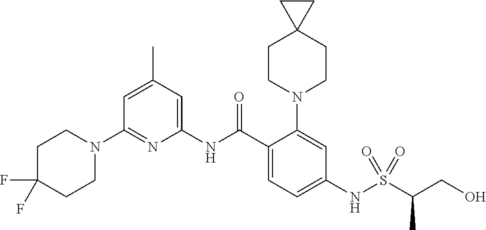 [SMILES: Cc1cc(NC(=O)c2ccc(NS(=O)(=O)[C@H](C)CO)cc2N2CCC3(CC2)CC3)nc(N2CCC(F)(F)CC2)c1] | (R)-N-(6-(4,4-Difluoropiperidin-1-yl)- 4-methylpyridin-2-yl)-4-((2-hydroxy-1- methylethyl)sulfonamido)-2-(6- azaspiro[2.5]octan-6-yl)benzamide |
| 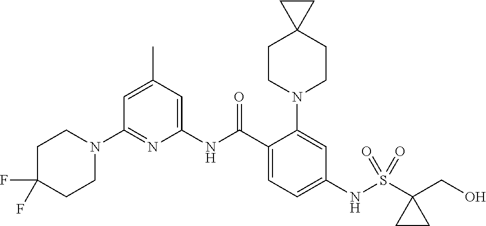 [SMILES: Cc1cc(NC(=O)c2ccc(NS(=O)(=O)C3(CO)CC3)cc2N2CCC3(CC2)CC3)nc(N2CCC(F)(F)CC2)c1] | N-(6-(4,4-Difluoropiperidin-1-yl)-4- methylpyridin-2-yl)-4-((1- (hydroxymethyl)cyclopropane)-1- sulfonamido)-2-(6-azaspiro[2.5]octan- 6-yl)benzamide |
| 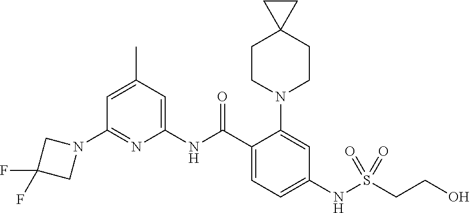 [SMILES: Cc1cc(NC(=O)c2ccc(NS(=O)(=O)CCO)cc2N2CCC3(CC2)CC3)nc(N2CC(F)(F)C2)c1] | N-(6-(3,3-Difluoroazetidin-1-yl)-4- methylpyridin-2-yl)-4-((2- hydroxyethyl)sulfonamido)-2-(6- azaspiro[2.5]octan-6-yl)benzamide |
| 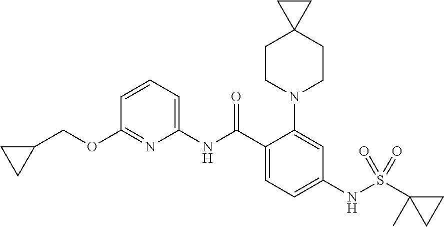 [SMILES: CC1(S(=O)(=O)Nc2ccc(C(=O)Nc3cccc(OCC4CC4)n3)c(N3CCC4(CC3)CC4)c2)CC1] | N-(6-(Cyclopropylmethoxy)pyridin-2- yl)-4-((1-methylcyclopropane)-1- sulfonamido)-2-(6-azaspiro[2.5]octan- 6-yl)benzamide |
| 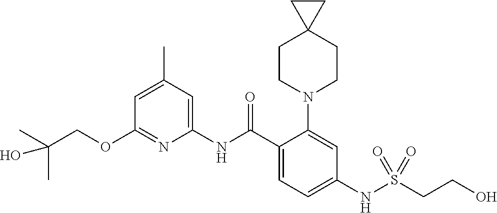 [SMILES: Cc1cc(NC(=O)c2ccc(NS(=O)(=O)CCO)cc2N2CCC3(CC2)CC3)nc(OCC(C)(C)O)c1] | N-(6-(2-Hydroxy-2-methylpropoxy)-4- methylpyridin-2-yl)-4-((2- hydroxyethyl)sulfonamido)-2-(6- azaspiro[2.5]octan-6-yl)benzamide |
| 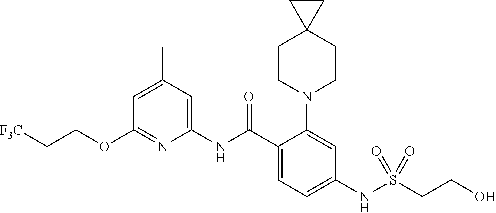 [SMILES: Cc1cc(NC(=O)c2ccc(NS(=O)(=O)CCO)cc2N2CCC3(CC2)CC3)nc(OCCC(F)(F)F)c1] | 4-((2-Hydroxyethyl)sulfonamido)-N-(4- methyl-6-(3,3,3- trifluoropropoxy)pyridin-2-yl)-2-(6- azaspiro[2.5]octan-6-yl)benzamide |
| 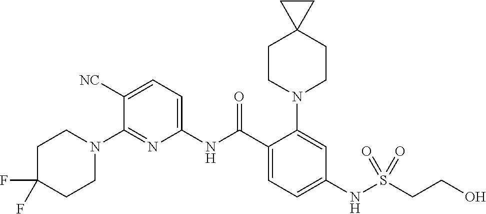 [SMILES: N#Cc1ccc(NC(=O)c2ccc(NS(=O)(=O)CCO)cc2N2CCC3(CC2)CC3)nc1N1CCC(F)(F)CC1] | N-(5-Cyano-6-(4,4-difluoropiperidin-1- yl)pyridin-2-yl)-4-((2- hydroxyethyl)sulfonamido)-2-(6- azaspiro[2.5]octan-6-yl)benzamide |
| 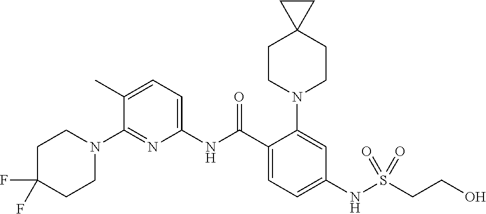 [SMILES: Cc1ccc(NC(=O)c2ccc(NS(=O)(=O)CCO)cc2N2CCC3(CC2)CC3)nc1N1CCC(F)(F)CC1] | N-(6-(4,4-Difluoropiperidin-1-yl)-5- methylpyridin-2-yl)-4-((2- hydroxyethyl)sulfonamido)-2-(6- azaspiro[2.5]octan-6-yl)benzamide |
| 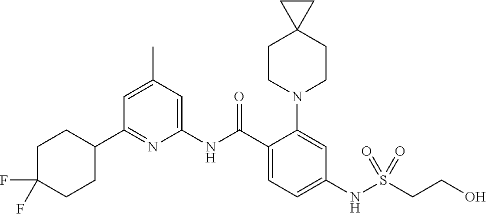 [SMILES: Cc1cc(NC(=O)c2ccc(NS(=O)(=O)CCO)cc2N2CCC3(CC2)CC3)nc(C2CCC(F)(F)CC2)c1] | N-(6-(4,4-difluorocyclohexyl)-4- methylpyridin-2-yl)-4-((2- hydroxyethyl)sulfonamido)-2-(6- azaspiro[2.5]octan-6-yl)benzamide |
| 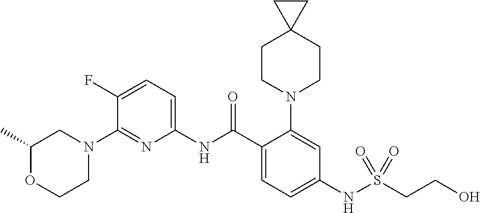 [SMILES: C[C@@H]1CN(c2nc(NC(=O)c3ccc(NS(=O)(=O)CCO)cc3N3CCC4(CC3)CC4)ccc2F)CCO1] | (R)-N-(5-Fluoro-6-(2- methylmorpholino)pyridin-2-yl)-4-((2- hydroxyethyl)sulfonamido)-2-(6- azaspiro[2.5]octan-6-yl)benzamide |
| 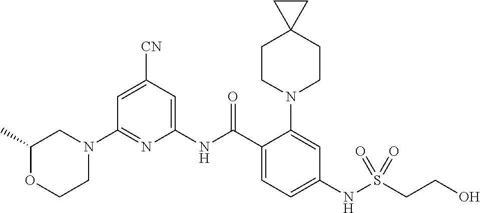 [SMILES: C[C@@H]1CN(c2cc(C#N)cc(NC(=O)c3ccc(NS(=O)(=O)CCO)cc3N3CCC4(CC3)CC4)n2)CCO1] | (R)-N-(4-Cyano-6-(2- methylmorpholino)pyridin-2-yl)-4-((2- hydroxyethyl)sulfonamido)-2-(6- azaspiro[2.5]octan-6-yl)benzamide |
| 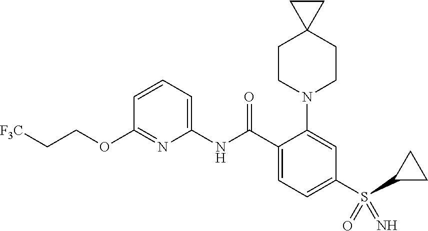 [SMILES: N=[S@](=O)(c1ccc(C(=O)Nc2cccc(OCCC(F)(F)F)n2)c(N2CCC3(CC2)CC3)c1)C1CC1] | 2-(6-Azaspiro[2.5]octan-6-yl)-4-(5- cyclopropylsulfonimidoyl)-N-(6-(3,3,3- trifluoropropoxy)-2- pyridinyl)benzamide |
| 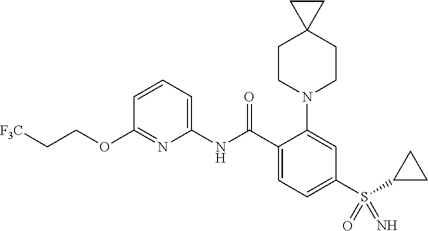 [SMILES: N=[S@@](=O)(c1ccc(C(=O)Nc2cccc(OCCC(F)(F)F)n2)c(N2CCC3(CC2)CC3)c1)C1CC1] | 2-(6-Azaspiro[2.5]octan-6-yl)-4-(R- cyclopropylsulfonimidoyl)-N-(6-(3,3,3- trifluoropropoxy)-2- pyridinyl)benzamide |

As used herein, the term “derivative thereof” refers to a salt thereof, a pharmaceutically acceptable salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, a geometric isomer thereof, a tautomer thereof, a mixture of tautomers thereof, an enantiomer thereof, a diastereomer thereof, a racemate thereof, a mixture of stereoisomers thereof, an isotope thereof (e.g., tritium, deuterium), or a combination thereof.

As used herein, the term “alkyl” refers to aliphatic hydrocarbon group which may be straight or branched. When not otherwise restricted, the term refers to an alkyl of 20 or fewer carbons. Branched means that one or more lower alkyl groups such as methyl, ethyl, or propyl are attached to a linear alkyl chain. Exemplary alkyl groups include methyl, ethyl, n-propyl, propyl, n-butyl, t-butyl, n-pentyl, 3-pentyl, and the like. The alkyl groups described in this section may also contain one or two double or triple bonds. A designation of C₀alkyl indicates a direct bond. Examples of C₁₋₆ alkyl include, but are not limited to the following:

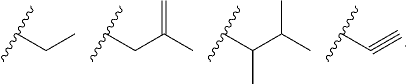

As used herein, “oxo” and “thioxo” represent the groups ═O (as in carbonyl) and ═S (as in thiocarbonyl), respectively.

As used herein, “halo” or “halogen” means a halogen atom selected from F, Cl, Br and I.

As used herein, “haloalkyl” refers to an alkyl group, as described above, wherein at least one of the hydrogen atoms attached to the alkyl chain are replaced by F, Cl, Br or I.

Small molecule KIF18A inhibitors can be readily modified using techniques known in the art to increase bioavailability (see Hetal et al, “A Review on Techniques for Oral Bioavailability Enhancement of Drugs,” Int'l. J. Pharm. Sci. Rev. Res. 4(3): 203-223 (2010) and Huttunen et al., “Prodrugs—from Serendipity to Rational Design,” Pharmacol. Rev. 63(3):750-771 (2011), which are hereby incorporated by reference in their entirety). For example, common modifications to increase the solubility and dissolution rate of small molecules include particle size reduction, modification of the crystal habit, dispersion in carriers, inclusion complexation, salt formation, and change in pH. Modification of the small molecule into a prodrug form using, for example, attached ionizable or polar neutral groups (e.g., phosphate esters, amino acids, sugar moieties) is also known to enhance solubility and dissolution rate. Common modification to increase permeability and absorption include, for example, conversion of hydrophilic hydroxyl, thiol, carboxyl, phosphate, or amine groups to more lipophilic alkyl or aryl esters.

In some embodiments, the KIF18A inhibitor is an anti-KIF18A antibody or KIF18A epitope binding fragment thereof.

Suitable KIF18A antibodies or binding fragments thereof include those that bind to an epitope within SEQ ID NO: 2. As used herein, “epitope” refers to the antigenic determinant of KIF18A that is recognized and bound by an antibody. The epitope recognized by the KIF18A antibody may be a linear epitope, i.e. the primary structure of the amino acid sequence of the isolated protein or peptide thereof. Alternatively, the epitope recognized by the KIF18A antibody or epitope binding portion thereof is a non-linear or conformational epitope. In all embodiments, the KIF18A antibody or epitope binding portion thereof recognizes and binds to a portion of KIF18A that blocks, inhibits, or reduces KIF18A activity. In one embodiment, the antibody binds to the neck linker domain or the enzymatic motor domain of KIF18A both located within amino acid residue 1-370 of SEQ ID NO: 2. In another embodiment, the antibody binds to the protein phosphatase 1 binding site within the C-terminal region of KIF18A, i.e., amino acid residues 612-616 of SEQ ID NO: 2.

Suitable KIF18A antibodies for use in accordance with the methods disclosed herein include any immunoglobulin molecule that specifically binds to a linear or conformational epitope of the KIF18A amino acid sequence of SEQ ID NO: 2 as defined herein. As used herein, the term “antibody” is meant to include intact immunoglobulins derived from natural sources or from recombinant sources, as well as immunoreactive portions (i.e., antigen binding portions) of intact immunoglobulins. Suitable KIF18A antibodies include, for example, polyclonal antibodies, monoclonal antibodies, intracellular antibodies (intrabodies), chimeric antibodies, and humanized antibodies.

Suitable KIF18A antibodies also include antibody fragments. Fragments of antibodies retaining binding activity include (i) Fab′ or Fab fragments, which are monovalent fragments containing the variable light (V_{L}) and variable heavy (V_{H}) chain regions, along with the light chain constant (C_{L}) region and a heavy chain constant region (C_{H}1); (ii) F(ab′)2 fragments, which are bivalent fragments comprising two Fab fragments linked by a disulfide bridge at the hinge region; (iii) Fd fragments consisting essentially of the V_{H} and C_{H}1 domains; (iv) Fv fragments consisting essentially of a V_{L} and V_{H} domain, (v) dAb fragments also called domain antibodies (Ward et al. “Binding Activities Of A Repertoire Of Single Immunoglobulin Variable Domains Secreted From Escherichia coli,” Nature 341:544-546 (1989) which is hereby incorporated by reference in its entirety), which consist essentially of a V_{H} or V_{L} domain (Holt et al. “Domain Antibodies: Proteins For Therapy,” Trends Biotechnol. 21(11):484-490 (2003), which is hereby incorporated by reference in its entirety); (vi) camelid or nanobodies (Revets et al. “Nanobodies as Novel Agents For Cancer Therapy,” Expert Opin. Biol. Ther. 5(1):111-124 (2005), which is hereby incorporated by reference in its entirety), (e.g. Fv, Fab and F(ab)2).

Suitable KIF18A antibodies also include antibody derivatives. Antibody derivatives include those molecules that contain at least one epitope-binding domain of an antibody, and are typically formed using recombinant techniques. One exemplary antibody derivative includes a single chain Fv (scFv). A scFv is formed from the two domains of the Fv fragment, the V_{L} region and the V_{H} region, which are encoded by separate gene. Such gene sequences or their encoding cDNA are joined, using recombinant methods, by a flexible linker (typically of about 10, 12, 15 or more amino acid residues) that enables them to be made as a single protein chain in which the V_{L} and V_{H} regions associate to form monovalent epitope-binding molecules (see e.g., Bird et al. “Single-Chain Antigen-Binding Proteins,” Science 242:423-426 (1988); and Huston et al. “Protein Engineering Of Antibody Binding Sites: Recovery Of Specific Activity In An Anti-Digoxin Single-Chain Fv Analogue Produced In Escherichia coli,” Proc. Natl. Acad. Sci. (U.S.A.) 85:5879-5883 (1988), which are hereby incorporated by reference in their entirety). Alternatively, by employing a flexible linker that is not too short (e.g., less than about 9 residues) to enable the V_{L} and V_{H} regions of a different single polypeptide chains to associate together, one can form a bispecific antibody, having binding specificity for two different epitopes.

Other suitable antibody derivatives include divalent or bivalent single-chain variable fragment, engineered by linking two scFvs together either in tandem (i.e., tandem scFv), or such that they dimerize to form diabodies (Holliger et al. “‘Diabodies’: Small Bivalent and Bispecific Antibody Fragments,” Proc. Natl. Acad. Sci. (U.S.A.) 90(14), 6444-8 (1993), which is hereby incorporated by reference in its entirety). In yet another embodiment, the antibody is a trivalent single chain variable fragment, engineered by linking three scFvs together, either in tandem or in a trimer formation to form triabodies. In another embodiment, the antibody is a tetrabody single chain variable fragment. In another embodiment, the antibody is a “linear antibody” which is an antibody comprising a pair of tandem Fd segments (V_{H}-C_{H}1-V_{H}-C_{H}1) that form a pair of antigen binding regions (see Zapata et al. Protein Eng. 8(10):1057-1062 (1995), which is hereby incorporated by reference in its entirety). In another embodiment, the antibody derivative is a minibody, consisting of the single-chain Fv regions coupled to the C_{H}3 region (i.e., scFv-C_{H}3).

In some embodiments, the KIF18A inhibitor is an inhibitory nucleic acid molecule, e.g., a KIF18A antisense RNA, shRNA, or siRNA oligonucleotide.

The use of antisense methods to inhibit the in vivo translation of genes and subsequent protein expression is well known in the art (e.g., U.S. Pat. No. 7,425,544 to Dobie et al.; U.S. Pat. No. 7,307,069 to Karras et al.; U.S. Pat. No. 7,288,530 to Bennett et al.; U.S. Pat. No. 7,179,796 to Cowsert et al., which are hereby incorporated by reference in their entirety). Antisense nucleic acids are nucleic acid molecules (e.g., molecules containing DNA nucleotides, RNA nucleotides, or modifications (e.g., modification that increase the stability of the molecule, such as 2′-O-alkyl (e.g., methyl) substituted nucleotides) or combinations thereof) that are complementary to, or that hybridize to, at least a portion of a specific nucleic acid molecule, such as an mRNA molecule (see e.g., Weintraub, H. M., “Antisense DNA and RNA,” Scientific Am. 262:40-46 (1990), which is hereby incorporated by reference in its entirety). The antisense nucleic acid molecule hybridizes to its corresponding target nucleic acid molecule, such as KIF18A, to form a double-stranded molecule, which interferes with translation of the mRNA, as the cell will not translate a double-stranded mRNA. Antisense nucleic acids used in the methods of the present invention are typically at least 10-12 nucleotides in length, for example, at least 15, 20, 25, 50, 75, or 100 nucleotides in length. The antisense nucleic acid can also be as long as the target nucleic acid with which it is intended to form an inhibitory duplex. Antisense nucleic acids can be introduced into cells as antisense oligonucleotides, or can be produced in a cell in which a nucleic acid encoding the antisense nucleic acid has been introduced, for example, using gene therapy methods.

siRNAs are double stranded synthetic RNA molecules approximately 20-25 nucleotides in length with short 2-3 nucleotide 3′ overhangs on both ends. The double stranded siRNA molecule represents the sense and anti-sense strand of a portion of the target mRNA molecule, in this case a portion of the KIF18A nucleotide sequence (SEQ ID NO: 1). siRNA molecules are typically designed to target a region of the mRNA target approximately 50-100 nucleotides downstream from the start codon. Upon introduction into a cell, the siRNA complex triggers the endogenous RNA interference (RNAi) pathway, resulting in the cleavage and degradation of the target mRNA molecule. Suitable siRNA molecules targeting the Kif18A sequence include, without limitation, GCCAAUUCUUCGUAGUUUU (SEQ ID NO: 3), GCAGCUGGAUUUCAUAAA (SEQ ID NO: 4) (Stumpff et al., “The Kinesin-8 Motor, Kif18A, Suppresses Kinetochore Movements to Control Mitotic Chromosome Alignment,” Dev. Cell 14(2): 252-262 (2008), which is hereby incorporated by reference in its entirety), GCCAAUUCUUCGUAGUUUUTT (SEQ ID NO: 5) and GCUGGAUUUCAUAAAGUGGTT (SEQ ID NO: 6) (Stumpff et al., “Kif18A and Chromokinesins Confine Centromere Movements via Microtubule Growth Suppression and Spatial Control of Kinetochore Tension,” Dev. Cell 22(5): 1017-1029 (2012), which is hereby incorporated by reference in its entirety). Various improvements of siRNA compositions, such as the incorporation of modified nucleosides or motifs into one or both strands of the siRNA molecule to enhance stability, specificity, and efficacy, have been described and are suitable for use in accordance with this aspect of the invention (see e.g., WO2004/015107 to Giese et al.; WO2003/070918 to McSwiggen et al.; WO1998/39352 to Imanishi et al.; U.S. Patent Application Publication No. 2002/0068708 to Jesper et al.; U.S. Patent Application Publication No. 2002/0147332 to Kaneko et al; U.S. Patent Application Publication No. 2008/0119427 to Bhat et al., which are hereby incorporated by reference in their entirety).

Short or small hairpin RNA molecules are similar to siRNA molecules in function, but comprise longer RNA sequences that make a tight hairpin turn. shRNA is cleaved by cellular machinery into siRNA and gene expression is silenced via the cellular RNA interference pathway. shRNA molecules that effectively interfere with KIF18A expression and are suitable for use in accordance with the methods described herein are known in the art, see e.g., Luo et al, “The Role of Kinesin KIF18A in the Invasion and Metastasis of Hepatocellular Carcinoma,” World J Surgical Oncol. 16:36 (2018), which is hereby incorporated by reference in its entirety.

In some embodiments, the method of inhibiting proliferation of chromosome instable cancer cells and treating cancer in a subject further involves administering, in conjunction with the KIF18A inhibitor, an agent that promotes microtubule turnover. In some embodiments, the agent is one that promotes kinetochore microtubule turnover. Agents that promote microtubule turnover, including kinetochore microtubule turnover, that are known in the art are suitable for use in accordance with this aspect of the disclosure.

In some embodiments, the agent that promotes microtubule turnover is an agent that enhances mitotic centromere-associated kinesin (MCAK; Kinsen-like protein KIF2C) activity. In some embodiments, the agent that enhances MCAK activity is a compound of Formula III

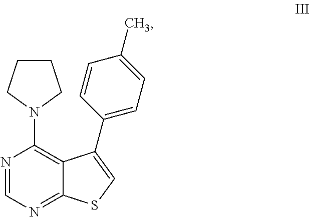 [SMILES: Cc1ccc(-c2csc3ncnc(N4CCCC4)c23)cc1]

or a derivative thereof,
also known as UMK57 (see Orr et al., “Adaptive Resistance to an Inhibitor of Chromosomal Instability in Human Cancer Cells,” Cell Reports 17(7):1755-1763 (2016), which is hereby incorporated by reference in its entirety). As demonstrated in the Examples herein, inhibition of KIF18A and small molecule activation (UMK57) of MCAK function synergistically inhibit tumor growth by disrupting mitotic progression and spindle bipolarity in CIN cells. Thus, in one embodiment, the methods of the present disclosure involve administering, to a subject having a cancer characterized by CIN, the combination of a KIF18A inhibitor and an agent that enhances mitotic centromere-associated kinesin (MCAK) activity.

In some embodiments, microtubule turnover is enhanced in the CIN cancer cells by delivering a nucleic acid molecule encoding MCAK (KIF2C) or a similar protein involved in promoting microtubule turnover to the cancer cells. Proteins known to promote microtubule turnover include, without limitation, KIF2A, KIF2B, Aurora B Kinase, and Aurora A Kinase. In accordance with this embodiment, gene therapy methods can be employed to deliver a polynucleotide sequence, i.e., a DNA or mRNA sequence, in a delivery vector or other suitable delivery vehicle to the cancer cells to effectuate protein expression and enhanced microtubule turnover activity.

In one embodiment, the nucleotide sequence delivered to CIN cancer cells in combination with a KIF18A inhibitor in accordance with the methods described herein is a polynucleotide sequence encoding MCAK (also known as kinesin-like protein KIF2C). Suitable mRNA and genomic sequences encoding MCAK are known in the art, see e.g., UniProt Accession No. Q99661. The mRNA sequence encoding isoform 1 of MCAK (the most prevalent sequence) is provided below as SEQ ID NO: 7 (NCBI Ref. Sequence NM_006845.3).

| (SEQ ID NO: 7) | |
|---|---|
| 1 | aaactgcggc ggtttacgcg gcgttaagac ttcgtagggt tagcgaaatt gaggtttctt |
| 61 | ggtattgcgc gtttctcttc cttgctgact ctccgaatgg ccatggactc gtcgcttcag |
| 121 | gcccgcctgt ttcccggtct cgctatcaag atccaacgca gtaatggttt aattcacagt |
| 181 | gccaatgtaa ggactgtgaa cttggagaaa tcctgtgttt cagtggaatg ggcagaagga |
| 241 | ggtgccacaa agggcaaaga gattgatttt gatgatgtgg ctgcaataaa cccagaactc |
| 301 | ttacagcttc ttcccttaca tccgaaggac aatctgccct tgcaggaaaa tgtaacaatc |
| 361 | cagaaacaaa aacggagatc cgtcaactcc aaaattcctg ctccaaaaga aagtcttcga |
| 421 | agccgctcca ctcgcatgtc cactgtctca gagcttcgca tcacggctca ggagaatgac |
| 481 | atggaggtgg agctgcctgc agctgcaaac tcccgcaagc agttttcagt tcctcctgcc |
| 541 | cccactaggc cttcctgccc tgcagtggct gaaataccat tgaggatggt cagcgaggag |
| 601 | atggaagagc aagtccattc catccgaggc agctcttctg caaaccctgt gaactcagtt |
| 661 | cggaggaaat catgtcttgt gaaggaagtg gaaaaaatga agaacaagcg agaagagaag |
| 721 | aaggcccaga actctgaaat gagaatgaag agagctcagg agtatgacag tagttttcca |
| 781 | aactgggaat ttgcccgaat gattaaagaa tttcgggcta ctttggaatg tcatccactt |
| 841 | actatgactg atcctatcga agagcacaga atatgtgtct gtgttaggaa acgcccactg |
| 901 | aataagcaag aattggccaa gaaagaaatt gatgtgattt ccattcctag caagtgtctc |
| 961 | ctcttggtac atgaacccaa gttgaaagtg gacttaacaa agtatctgga gaaccaagca |
| 1021 | ttctgctttg actttgcatt tgatgaaaca gcttcgaatg aagttgtcta caggttcaca |
| 1081 | gcaaggccac tggtacagac aatctttgaa ggtggaaaag caacttgttt tgcatatggc |
| 1141 | cagacaggaa gtggcaagac acatactatg ggcggagacc tctctgggaa agcccagaat |
| 1201 | gcatccaaag ggatctatgc catggcctcc cgggacgtct tcctcctgaa gaatcaaccc |
| 1261 | tgctaccgga agttgggcct ggaagtctat gtgacattct tcgagatcta caatgggaag |
| 1321 | ctgtttgacc tgctcaacaa gaaggccaag ctgcgcgtgc tggaggacgg caagcaacag |
| 1381 | gtgcaagtgg tggggctgca ggagcatctg gttaactctg ctgatgatgt catcaagatg |
| 1441 | atcgacatgg gcagcgcctg cagaacctct gggcagacat ttgccaactc caattcctcc |
| 1501 | cgctcccacg cgtgcttcca aattattctt cgagctaaag ggagaatgca tggcaagttc |
| 1561 | tctttggtag atctggcagg gaatgagcga ggcgcggaca cttccagtgc tgaccggcag |
| 1621 | acccgcatgg agggcgcaga aatcaacaag agtctcttag ccctgaagga gtgcatcagg |
| 1681 | gccctgggac agaacaaggc tcacaccccg ttccgtgaga gcaagctgac acaggtgctg |
| 1741 | agggactcct tcattgggga gaactctagg acttgcatga ttgccacgat ctcaccaggc |
| 1801 | ataagctcct gtgaatatac tttaaacacc ctgagatatg cagacagggt caaggagctg |
| 1861 | agcccccaca gtgggcccag tggagagcag ttgattcaaa tggaaacaga agagatggaa |
| 1921 | gcctgctcta acggggcgct gattccaggc aatttatcca aggaagagga ggaactgtct |
| 1981 | tcccagatgt ccagctttaa cgaagccatg actcagatca gggagctgga ggagaaggct |
| 2041 | atggaagagc tcaaggagat catacagcaa ggaccagact ggcttgagct ctctgagatg |
| 2101 | accgagcagc cagactatga cctggagacc tttgtgaaca aagcggaatc tgctctggcc |
| 2161 | cagcaagcca agcatttctc agccctgcga gatgtcatca aggccttgcg cctggccatg |
| 2221 | cagctggaag agcaggctag cagacaaata agcagcaaga aacggcccca gtgacgactg |
| 2281 | caaataaaaa tctgtttggt ttgacaccca gcctcttccc tggccctccc cagagaactt |
| 2341 | tgggtacctg gtgggtctag gcagggtctg agctgggaca ggttctggta aatgccaagt |
| 2401 | atgggggcat ctgggcccag ggcagctggg gagggggtca gagtgacatg ggacactcct |
| 2461 | tttctgttcc tcagttgtcg ccctcacgag aggaaggagc tcttagttac ccttttgtgt |
| 2521 | tgcccttctt tccatcaagg ggaatgttct cagcatagag ctttctccgc agcatcctgc |
| 2581 | ctgcgtggac tggctgctaa tggagagctc cctggggttg tcctggctct ggggagagag |
| 2641 | acggagcctt tagtacagct atctgctggc tctaaacctt ctacgccttt gggccgagca |
| 2701 | ctgaatgtct tgtactttaa aaaaatgttt ctgagacctc tttctacttt actgtctccc |
| 2761 | tagagatcct agaggatccc tactgttttc tgttttatgt gtttatacat tgtatgtaac |
| 2821 | aataaagaga aaaaataaat cagctgttta agtgtgtgga aa |

In one embodiment, the nucleotide sequence delivered to CIN cancer cells in combination with the KIF18A inhibitor in accordance with the methods described herein is a polynucleotide sequence encoding Kinesin-like protein KIF2A (Kinesin-2). Suitable mRNA and genomic sequences encoding Kinesin-2 are known in the art, see e.g., UniProt Accession No. O00139. The mRNA sequence encoding isoform 3 of Kinesin-2 (the most prevalent sequence) is provided herein as SEQ ID NO: 8 (NCBI Ref. Sequence: NM_004520.5)

| (SEQ ID NO: 8) | |
|---|---|
| 1 | actctacccc gcgccgtctc acggccccgg ccctagcttc accccgacta cccggcgtgc |
| 61 | gcgtcctcct gccggcctgc aggcccgggg cctccgcctg cttccccaca gctgctcctt |
| 121 | gcggccccgc ttgcgttcac gctgtcgccc gggccggcgc ggccgcgggc aaccgctccc |
| 181 | cctcccacac ctaccccgcc ccctccccgc cttttccgcc ctccggtccc cctccctcgg |
| 241 | cccgctgctg ctgctccaga tgaggtgatg gcaacggcca acttcggcaa gatccagatc |
| 301 | gggatttacg tggagatcaa gcgcagcgat ggccgaatac atcaagcaat ggtaacatct |
| 361 | ttaaatgaag ataatgaaag tgtaactgtt gaatggatag aaaatggaga tacaaaaggc |
| 421 | aaagagattg acctggagag catcttttca cttaaccctg accttgttcc tgatgaagaa |
| 481 | attgaaccca gtccagaaac acctccacct ccagcatcct cagccaaagt aaacaaaatt |
| 541 | gtaaagaatc gacggactgt agcttctatt aagaatgacc ctccttcaag agataataga |
| 601 | gtggttggtt cagcacgtgc acggcccagt caatttcctg aacagtcttc ctctgcacaa |
| 661 | cagaatggta gtgtttcaga tatatctcca gttcaagctg caaaaaagga atttggaccc |
| 721 | ccttcacgta gaaaatctaa ttgtgtgaaa gaagtagaaa aactgcaaga aaaacgagag |
| 781 | aaaaggagat tgcaacagca agaacttaga gaaaaaagag cccaggacgt tgatgctaca |
| 841 | aacccaaatt atgaaattat gtgtatgatc agagacttta gaggaagttt ggattataga |
| 901 | ccattaacaa cagcagatcc tattgatgaa cataggatat gtgtgtgtgt aagaaaacga |
| 961 | ccactcaata aaaaagaaac tcaaatgaaa gatcttgatg taatcacaat tcctagtaaa |
| 1021 | gatgttgtga tggtacatga accaaaacaa aaagtagatt taacaaggta cctagaaaac |
| 1081 | caaacatttc gttttgatta tgcctttgat gactcagctc ctaatgaaat ggtttacagg |
| 1141 | tttactgcta gaccactagt ggaaactata tttgaaaggg gaatggctac atgctttgct |
| 1201 | tatgggcaga ctggaagtgg aaaaactcat actatgggtg gtgacttttc aggaaagaac |
| 1261 | caagattgtt ctaaaggaat ttatgcatta gcagctcgag atgtcttttt aatgctaaag |
| 1321 | aagccaaact ataagaagct agaacttcaa gtatatgcaa ccttctttga aatttatagt |
| 1381 | ggaaaggtgt ttgacttgct aaacaggaaa acaaaattaa gagttctaga agatggaaaa |
| 1441 | cagcaggttc aagtggtggg attacaggaa cgggaggtca aatgtgttga agatgtactg |
| 1501 | aaactcattg acataggcaa cagttgcaga acatccggtc aaacatctgc aaatgcacat |
| 1561 | tcatctcgga gccatgcagt gtttcagatt attcttagaa ggaaaggaaa actacatggc |
| 1621 | aaattttctc tcattgattt ggctggaaat gaaagaggag ctgatacttc cagtgcggac |
| 1681 | aggcaaacta ggcttgaagg tgctgaaatt aataaaagcc ttttagcact caaggagtgc |
| 1741 | atcagagcct taggtagaaa taaacctcat actcctttcc gtgcaagtaa actcactcag |
| 1801 | gtgttaagag attctttcat aggtgaaaac tctcgtacct gcatgattgc cacaatctct |
| 1861 | ccaggaatgg catcctgtga aaatactctt aatacattaa gatatgcaaa tagggtcaaa |
| 1921 | gaattgactg tagatccaac tgctgctggt gatgttcgtc caataatgca ccatccacca |
| 1981 | aaccagattg atgacttaga gacacagtgg ggtgtgggga gttcccctca gagagatgat |
| 2041 | ctaaaacttc tttgtgaaca aaatgaagaa gaagtctctc cacagttgtt tactttccac |
| 2101 | gaagctgttt cacaaatggt agaaatggaa gaacaagttg tagaagatca cagggcagtg |
| 2161 | ttccaggaat ctattcggtg gttagaagat gaaaaggccc tcttagagat gactgaagaa |
| 2221 | gtagattatg atgtcgattc atatgctaca caacttgaag ctattcttga gcaaaaaata |
| 2281 | gacattttaa ctgaactgcg ggataaagtg aaatctttcc gtgcagctct acaagaggag |
| 2341 | gaacaagcca gcaagcaaat caacccgaag agaccccgtg ccctttaaac cggcatttgc |
| 2401 | tgctaaagga tacccagaac cctcactact gtaacataca acggttcagc tgtaagggcc |
| 2461 | atttgaaagt ttggaatttt aagtgtctgt ggaaaatgtt ttgtccttca cctgaattac |
| 2521 | atttcaattt tgtgaaacac tcttttgtct acaaaatgct tctagtccag gaggcacaac |
| 2581 | caagaactgg gattaatgaa gcattttgtt tcatttacac aaatagtgat ttacttttgg |
| 2641 | agatccttgt cagttttatt ttctatttga tgaagtaaga ctgtggactc aatccagagc |
| 2701 | cagatagtag ggggaagcca cagcatttcc ttttaactca gttcaatttt tgtagtgaga |
| 2761 | ctgagcagtt ttaaatcctt tgcgtgcatg catacctcat cagtgattgt acataccttg |
| 2821 | cccactccta gagacagctg tgctcacctt ttcctgcttt gtgccttgat taaggctact |
| 2881 | gaccctaaat ttctgaagca cagccaagaa aaattacatt ccttgtcatt gtaaattacc |
| 2941 | tttgtgtgta catttttact gtatttgaga cattttttgt gtgtgactag ttaattttgc |
| 3001 | aggatgtgcc atatcattga acggaactaa agtctgtgac agtggatata gctgctggac |
| 3061 | cattccatct tatatgtaaa gaaatctgga attattattt taaaaccata taacatgtga |
| 3121 | ttataatttt tcttagcatt ttctttgtaa agaactacaa tataaactag ttggtgtata |
| 3181 | ataaaaagta atgaaattct gagaagagtt ttatcttagg aaaatacata tatatgcagt |
| 3241 | gtgtgtgcca gtgtggtatt aacaagacta atagtgcagt ttgatcctta ccaatatcat |
| 3301 | tacttaattt gaagtgttca ttagcacccc aaaatatacc ttttctatgt actgttaaaa |
| 3361 | gaaattggct tctgatgcat gaacatttac atgtacattg aaagtagtcc ataatagaag |
| 3421 | ttagtttaag ccaagtgtag acagtacatt actcccttga aaaagaatta agttgaaaga |
| 3481 | gttgactttg ccttaaaagg cagatctaac ccaagctcca tccagtacca aatgtgaaac |
| 3541 | ttcattgttg tttggtgaga atcgccaaat tctcactaat acattggtat ggtgtagggc |
| 3601 | atgctacttt ttaaacagca gcacttattt ttacagattg ctactccaag gaagaaaact |
| 3661 | ggccactttt catgtaaata ttttgttcaa agatttgtat atctctctag gagttttccc |
| 3721 | tcagttccca ggatggggtc caggagtaga ttaacagcta aaaatctccc aaggatatct |
| 3781 | tgttttgatt tatttactcc agggaactat cagctccttc acaggagcca aaggggagct |
| 3841 | atgaatagag ggtcacatga gccagattct ttactgttca taaaacccag agagtagttg |
| 3901 | tgaaagatcc taatacaatt gtgaaaagct ctgtaaacat gaaatctaaa acaaatgtag |
| 3961 | attttcacaa tatgcttatt aataaatgaa gtctatggaa taagttttag agataattta |
| 4021 | cttcagtcac ctttgttttg gaaaggactc aggttttctt gcagctgtaa gatatattct |
| 4081 | atttgtgttt atttcaaagg gaagaggaag gatggagaac tgttacaatt gaccttgcaa |
| 4141 | aggatattta aaaaaaaggt agtttttgag attaaccaac tttcaaaggg caataaagac |
| 4201 | atgtgaattt gctcatttta aagcacaaca gatgattagc ttcaaattta ttatttgcat |
| 4261 | tggatgggta ctgtctttgg aaagtctcct tatagacaaa tatgctgcct tacactatga |
| 4321 | tggcttcatt ctgatcaggt attttaaaaa ttagtaccag aaaagatact ggaggtaata |
| 4381 | taatacagtc cttcagcttt acagatagtg aaaactgaag gccagaaaag gaactaaaac |
| 4441 | tcagcagttc ataggggtag agggaaataa cctgagaaag ccgagttaaa tcttaaaatt |
| 4501 | tttactataa ggatagagat gatacaagtg aactctgcaa aatagttttg tgaaattaaa |
| 4561 | caaaaaaatc tactcttaat gtatattatt tcatatttgt ttaacaaaag cagcttgatg |
| 4621 | cctttgttct ggattataca ttaagaacaa gcattatttt aattatgtag taacatttac |
| 4681 | tatgactttg aagccagatt tcaaaatctg gctttgttaa attaattgga aacactcttc |
| 4741 | tattaagtta ggtattaaaa aaagccaaat atcaataaag atatttttat taatttttta |
| 4801 | tagaaacaaa atagctacta taactctatt atagtatatt aacagcactt agtattatat |
| 4861 | tgacttaaac atagtagcta ataaaactag tttatgacat ctattgaaat tctcagtctg |
| 4921 | acagttttaa aatagcttaa aactaagcac caggtatatg aaaaaggaac tggaaatttt |
| 4981 | aattgtccag aaatcatagg aatactggga acctgtagtt atgggaaatg gctgtttttc |
| 5041 | tgaagttgaa accagtttca attacaaaac cctctgaaat tttgctaagc ctatagttat |
| 5101 | ctccctaaga accataaaaa aagataattt tattgttaat ttcattccag tttagcttcc |
| 5161 | tcattttaca tgttgggagt catgcccttt aatatgcctg gcactgcacg gtggcagtag |
| 5221 | cagacttggg cttacccaga accctaagtc tctgacgcta taggatagcg aaagaggtag |
| 5281 | gcaatcgtaa cagacactgt ataggataaa tagggaagag aatgagagaa ccaaaataaa |
| 5341 | aataatttaa gaaacaattg ttttactgta catgtgaata acaagaaatg gtacggggaa |
| 5401 | tgtgaataac acgaaatggt atggggaatg tgtgactagt gaagcatatt ttaacttttt |
| 5461 | tattactgga ggaacagagc taaaggccta aagaaggcct tacagtatat aacagtaaat |
| 5521 | agaagagtaa catctttata caataaactg ttagaaatga taagtgtaaa gttgtgcgtc |
| 5581 | tctgcggctc ctgaacttga agattattat gaatagattg gaccagcatt atatattaaa |
| 5641 | aactttgata cttagaactt tccaacttta aaattcagaa tcataaatgg tgacaacagt |
| 5701 | agtagtattg aaccaaaaat agtcaagtaa ataatgtctc agtaaagcaa aagcattatc |
| 5761 | ttctcaaata caaaaaatac aaaattcatt tcttttcttg accttgaaaa tttctgtttt |
| 5821 | ccaaatacct aggaaaaatg aataccttct gcgttgaatc catgtagcaa tctgaaaaaa |
| 5881 | gaaatcaaaa tggaatggta ctgaaaagct aatttgtagc acataacggt atatagttct |
| 5941 | ataccattaa tactaaaaca tgaatacagc atgcttacag agcccaccca ctcctaatac |
| 6001 | tagttattaa agaaacgtta ctggctgggc gcagtggctt atgcctgtaa tcccagcact |
| 6061 | ttgggaggct gaggcgggtg gattacctga ggtcagtagt tcgagaacag tctggccaac |
| 6121 | atggtgaaac cccgtctcta ctaaaaatac aaaaattagc ctggcatggt ggcacatgcc |
| 6181 | tgtaatccca gctactcggg tggttgaggc aggagatttg cctgaaccca ggaggcggag |
| 6241 | gttgcagtga gctgagattg tgccactgga gtccagcctg ggtgacagag caagactctg |
| 6301 | tctcaaaaaa aaaaaaaaaa gaaatgttat tgtctgacta aatttataag atatgtattg |
| 6361 | gttaaaacca tgccatgttg gtgcctcttt cattagagcc tttatgtcgt aatgaatttg |
| 6421 | actaaaatta tcttagactt tcattatccc aggcctaaga actcactggc atttgtttca |
| 6481 | aggtaactga acaagaagct gtttggaatt ggcagaagtc agatgaaaaa ccaatcttac |
| 6541 | atgccccttc ttcctctctt gagctgttgt ttatattcaa attaaataca cattgtttct |
| 6601 | ctctgtagat acctatgtac ttaatagatt ctagttagta aactgcacat gcccaataac |
| 6661 | tttgaggaaa tttagtgaaa atgaagaaaa agagaaaata tttctcttta gacctgaggt |
| 6721 | tatgtttagg ctggcccata gaaacaggtc cagataaatt tctaaaaaag caaagtagat |
| 6781 | atttatgaat agtattcaat gcctaggatt aacatctaaa atgactcagt agtactgcta |
| 6841 | gccagccaat aaaatataaa ctccatttgt cttagttata tagaactgtg tttccagctt |
| 6901 | agaaaaagtc aaaccaatga cttttagaac aatctactct cattttttat tcagcctcta |
| 6961 | gaacatggaa gctttaaaag tgaattggct aaataggcaa gaccttctga aagttaacat |
| 7021 | cttaatgatt aaaaacagta agtacaggtt agtaattacc tgggtaatta attgaagcct |
| 7081 | tattctgttt tcataagact tacttgctta attcaagcaa aacaaatttt ggtctaaatt |
| 7141 | acctagataa ttatgacagc tttttacttg agaagtgtag aacttgcttc aggctacaaa |
| 7201 | actgtattat tcctaaatgg ataaccaggt aggattctaa ctggcattat tgtatgctta |
| 7261 | agattgattt aacaacagct attcccagta aggaaatttt aaaaatcaga tccagttaca |
| 7321 | tgtattatga tttttctacc ttatggacta ttttggaggg ataagctatt aagactaaga |
| 7381 | ctatgaatga gagttgggga aggagcagga agggaggaac ctgcacacca cattggaacc |
| 7441 | tgcacaccac attaacacaa aggcaatctt ctggctcgga ctgttcttta ctactgttct |
| 7501 | taaagaaaat gttcattctg ctgcagctaa ctagcctcca tcttctacac caaatactat |
| 7561 | tccatgccat ggaagtgcta tgcaataact ctcccaggta gcaccttata ccgcttaaaa |
| 7621 | gcctttaaaa tctccaatct gaaggtgtca cagtaaagaa atgtaaacac ttaggaaaac |
| 7681 | aaaaatgtaa ttacctgatg aagtcatcta tgtccatgga acgggcccgt ttgtcactaa |
| 7741 | aacctgtgct ggttaggatt tgctgtattt tatctgctat gctgaaatct tctggtatta |
| 7801 | tctatcaata taagattcag aataaatgaa cgacatatct ttaa |

In one embodiment, the nucleotide sequence delivered to CIN cancer cells in combination with a KIF18A inhibitor in accordance with the methods described herein is a polynucleotide sequence encoding Kinesin-like protein KIF2B. Suitable mRNA and genomic sequences encoding Kinesin-like protein KIF2B are known in the art, see e.g., UniProt Accession No. Q8N4N8. The mRNA sequence encoding Kinesin-like protein KIF2B is 40 provided below as SEQ ID NO: 9 (NCBI Reference Sequence NM_032559.4).

| (SEQ ID NO: 9) | |
|---|---|
| 1 | gtagtggccc cagtccgggc cccggcgcgc taggctcaca aaggcaggca cagactgcaa |
| 61 | ccctgctcag tgctccgggc gcttcaggct ggcttgggtc ctgctgctcc aaccccaagg |
| 121 | gccctggagc gctccctgat acctccatca ctcaccatgg ccagccagtt ctgcctccct |
| 181 | gaatccccat gtctctcgcc cctgaaaccc ttgaagccac atttcggaga catccaagag |
| 241 | ggcatctacg tggcgatcca gcgcagtgac aagcggatcc acctcgctgt ggtcacggag |
| 301 | atcaacagag aaaactattg ggtcacggta gagtgggtgg agaaagcagt caaaaaaggc |
| 361 | aagaagattg acctggagac catactcctg ctgaatccag ctctggactc tgctgaacac |
| 421 | cccatgccgc ccccgccctt atcccccttg gctctggcgc cctcttcggc catcagggac |
| 481 | cagcgtaccg ccacgaaatg ggttgcgatg atcccccaga aaaaccaaac agcctcaggg |
| 541 | gacagcctgg atgtgagggt ccccagcaaa ccttgtctga tgaagcagaa aaagtctccc |
| 601 | tgcctctggg aaatccagaa actgcaggag cagcgggaaa agcgcaggcg gctgcagcag |
| 661 | gagatccgag ctagacgcgc cctcgatgtc aataccagaa accccaacta cgaaatcatg |
| 721 | cacatgatcg aagagtatcg caggcacctg gacagcagca agatctcagt cctggagccc |
| 781 | ccgcaagaac atcgcatctg cgtctgcgtg aggaagcggc ctctcaacca gcgagagaca |
| 841 | accttaaagg acctggatat catcaccgtc ccctcggaca atgtggttat ggtgcatgag |
| 901 | tccaagcaaa aggtggacct cactcgctac ctgcagaacc agaccttctg cttcgaccat |
| 961 | gccttcgatg acaaagcctc caacgagttg gtgtaccagt tcaccgccca gccactggtg |
| 1021 | gagtccatct tccgcaaggg catggccacc tgctttgcct atgggcagac gggaagtggg |
| 1081 | aagacgtaca ccatgggtgg agacttttca ggaacggccc aagattgttc taagggcatt |
| 1141 | tatgctctgg tggcacagga tgtctttctc ctgctcagaa actccacata tgagaagctg |
| 1201 | gacctcaaag tctatgggac attttttgag atttatgggg gcaaggtgta tgatttgttg |
| 1261 | aactggaaga agaagctgca agtccttgag gatggcaatc agcaaatcca agtggtcggg |
| 1321 | ctgcaggaga aagaggtgtg ttgtgtggag gaagtgctga acctggtgga aatagggaat |
| 1381 | agctgtcgga cttccaggca aacacctgtc aacgctcact catccaggag ccatgcagtg |
| 1441 | ttccagatca tcctgaagtc aggacggata atgcatggca agttttccct cgttgattta |
| 1501 | gctgggaatg aaagaggagc agatacaacc aaggccagcc ggaaaaggca gctggaaggg |
| 1561 | gcagagatta acaagagtct tctagccctc aaagaatgta ttctggcttt gggtcagaac |
| 1621 | aagcctcaca ccccattcag agccagcaaa ctcacactgg tgctccggga ctcctttata |
| 1681 | ggccagaact cctccacttg catgattgct accatctctc cggggatgac ctcttgtgaa |
| 1741 | aacactctca acactttaag atatgcaaac agagtaaaaa aattaaatgt agatgtaagg |
| 1801 | ccctaccatc gtggccacta tccgattgga catgaggcac caaggatgtt aaaaagtcac |
| 1861 | atcggaaatt cagaaatgtc ccttcagagg gatgaattta ttaaaatacc ttatgtacag |
| 1921 | agtgaggagc agaaagagat tgaagaggtt gaaacattac ccactctgtt agggaaggat |
| 1981 | accacaattt cagggaaggg atctagccaa tggctggaaa acatccagga gagagctggt |
| 2041 | ggagtacacc atgatattga tttttgcatt gcccggtctt tgtccatttt ggagcagaaa |
| 2101 | attgatgctc tgaccgagat ccaaaagaaa ctgaaattat tactagctga cctccacgtg |
| 2161 | aagagcaagg tagagtgaag ccaatggcga gagatcaggt ccgaaatgct gcattgctgc |
| 2221 | agtttccacc actcttatac aggaaaactg tccaaattat ctaaagatcc tcctgagaag |
| 2281 | cttaaaacat cttaaaatac actgatggga aacatgctct ttcttctgcc tctgt |

In one embodiment, the nucleotide sequence delivered to CIN cancer cells in conjunction with a KIF18A inhibitor in accordance with the methods described herein is a polynucleotide sequence encoding Aurora B Kinase (Aurora 1). Suitable mRNA and genomic sequences encoding Aurora B Kinase are known in the art, see e.g., UniProt Accession No. Q96GD4. The mRNA sequence encoding isoform 1 of Aurora B Kinase (the most prevalent sequence) is provided below as SEQ ID NO: 10 (NCBI Reference Sequence No. NM_001313950).

| (SEQ ID NO: 10) | |
|---|---|
| 1 | agttgtttgc gggcggccgg gagagtagca gtgccttgga ccccaggatg gcccagaagg |
| 61 | agaactccta cccctggccc tacggccgac agacggctcc atctggcctg agcaccctgc |
| 121 | cccagcgagt cctccggaaa gagcctgtca ccccatctgc acttgtcctc atgagccgct |
| 181 | ccaatgtcca gcccacagct gcccctggcc agaaggtgat ggagaatagc agtgggacac |
| 241 | ccgacatctt aacgcggcac ttcacaattg atgactttga gattgggcgt cctctgggca |
| 301 | aaggcaagtt tggaaacgtg tacttggctc gggagaagaa aagccatttc atcgtggcgc |
| 361 | tcaaggtcct cttcaagtcc cagatagaga aggagggcgt ggagcatcag ctgcgcagag |
| 421 | agatcgaaat ccaggcccac ctgcaccatc ccaacatcct gcgtctctac aactattttt |
| 481 | atgaccggag gaggatctac ttgattctag agtatgcccc ccgcggggag ctctacaagg |
| 541 | agctgcagaa gagctgcaca tttgacgagc agcgaacagc cacgatcatg gaggagttgg |
| 601 | cagatgctct aatgtactgc catgggaaga aggtgattca cagagacata aagccagaaa |
| 661 | atctgctctt agggctcaag ggagagctga agattgctga cttcggctgg tctgtgcatg |
| 721 | cgccctccct gaggaggaag acaatgtgtg gcaccctgga ctacctgccc ccagagatga |
| 781 | ttgaggggcg catgcacaat gagaaggtgg atctgtggtg cattggagtg ctttgctatg |
| 841 | agctgctggt ggggaaccca ccctttgaga gtgcatcaca caacgagacc tatcgccgca |
| 901 | tcgtcaaggt ggacctaaag ttccccgctt ccgtgcccat gggagcccag gacctcatct |
| 961 | ccaaactgct caggcataac ccctcggaac ggctgcccct ggcccaggtc tcagcccacc |
| 1021 | cttgggtccg ggccaactct cggagggtgc tgcctccctc tgcccttcaa tctgtcgcct |
| 1081 | gatggtccct gtcattcact cgggtgcgtg tgtttgtatg tctgtgtatg tataggggaa |
| 1141 | agaagggatc cctaactgtt cccttatctg ttttctacct cctcctttgt ttaataaagg |
| 1201 | ctgaagcttt ttgtactca |

In one embodiment, the nucleotide sequence delivered to CIN cancer cells in conjunction with a KIF18A inhibitor in accordance with the methods described herein is a polynucleotide sequence encoding Aurora A Kinase (Aurora 2). Suitable mRNA and genomic sequences encoding Aurora A Kinase are known in the art, see e.g., UniProt Accession No. 014965. The mRNA sequence encoding Aurora A Kinase (variant 1) is provided below as SEQ ID NO: 11 (NCBI Ref. Sequence NM_198433.3).

| (SEQ ID NO: 11) | |
|---|---|
| 1 | gaattctaac ggctgagctc ttggaagact tgggtccttg ggtcgcaggt gggagccgac |
| 61 | gggtgggtag accgtggggg atatctcagt ggcggacgag gacggcgggg acaaggggcg |
| 121 | gctggtcgga gtggcggagc gtcaagtccc ctgtcggttc ctccgtccct gagtgtcctt |
| 181 | ggcgctgcct tgtgcccgcc cagcgccttt gcatccgctc ctgggcaccg aggcgccctg |
| 241 | taggatactg cttgttactt attacagcta gagggtctca ctccattgcc caggccagag |
| 301 | tgcggggata tttgataaga aacttcagtg aaggccgggc gcggtggctc atgcccgtaa |
| 361 | tcccagcatt ttcggaggcc gaggctggag tgcaatggtg tgatctcagc tcactgcaac |
| 421 | ctctgcttcc tgggtttaag tgattctcct gcctcagcct cccgagtagc tgggattaca |
| 481 | ggcatcatgg accgatctaa agaaaactgc atttcaggac ctgttaaggc tacagctcca |
| 541 | gttggaggtc caaaacgtgt tctcgtgact cagcaatttc cttgtcagaa tccattacct |
| 601 | gtaaatagtg gccaggctca gcgggtcttg tgtccttcaa attcttccca gcgcattcct |
| 661 | ttgcaagcac aaaagcttgt ctccagtcac aagccggttc agaatcagaa gcagaagcaa |
| 721 | ttgcaggcaa ccagtgtacc tcatcctgtc tccaggccac tgaataacac ccaaaagagc |
| 781 | aagcagcccc tgccatcggc acctgaaaat aatcctgagg aggaactggc atcaaaacag |
| 841 | aaaaatgaag aatcaaaaaa gaggcagtgg gctttggaag actttgaaat tggtcgccct |
| 901 | ctgggtaaag gaaagtttgg taatgtttat ttggcaagag aaaagcaaag caagtttatt |
| 961 | ctggctctta aagtgttatt taaagctcag ctggagaaag ccggagtgga gcatcagctc |
| 1021 | agaagagaag tagaaataca gtcccacctt cggcatccta atattcttag actgtatggt |
| 1081 | tatttccatg atgctaccag agtctaccta attctggaat atgcaccact tggaacagtt |
| 1141 | tatagagaac ttcagaaact ttcaaagttt gatgagcaga gaactgctac ttatataaca |
| 1201 | gaattggcaa atgccctgtc ttactgtcat tcgaagagag ttattcatag agacattaag |
| 1261 | ccagagaact tacttcttgg atcagctgga gagcttaaaa ttgcagattt tgggtggtca |
| 1321 | gtacatgctc catcttccag gaggaccact ctctgtggca ccctggacta cctgccccct |
| 1381 | gaaatgattg aaggtcggat gcatgatgag aaggtggatc tctggagcct tggagttctt |
| 1441 | tgctatgaat ttttagttgg gaagcctcct tttgaggcaa acacatacca agagacctac |
| 1501 | aaaagaatat cacgggttga attcacattc cctgactttg taacagaggg agccagggac |
| 1561 | ctcatttcaa gactgttgaa gcataatccc agccagaggc caatgctcag agaagtactt |
| 1621 | gaacacccct ggatcacagc aaattcatca aaaccatcaa attgccaaaa caaagaatca |
| 1681 | gctagcaaac agtcttagga atcgtgcagg gggagaaatc cttgagccag ggctgccata |
| 1741 | taacctgaca ggaacatgct actgaagttt attttaccat tgactgctgc cctcaatcta |
| 1801 | gaacgctaca caagaaatat ttgttttact cagcaggtgt gccttaacct ccctattcag |
| 1861 | aaagctccac atcaataaac atgacactct gaagtgaaag tagccacgag aattgtgcta |
| 1921 | cttatactgg ttcataatct ggaggcaagg ttcgactgca gccgccccgt cagcctgtgc |
| 1981 | taggcatggt gtcttcacag gaggcaaatc cagagcctgg ctgtggggaa agtgaccact |
| 2041 | ctgccctgac cccgatcagt taaggagctg tgcaataacc ttcctagtac ctgagtgagt |
| 2101 | gtgtaactta ttgggttggc gaagcctggt aaagctgttg gaatgagtat gtgattcttt |
| 2161 | ttaagtatga aaataaagat atatgtacag acttgtattt tttctctggt ggcattcctt |
| 2221 | taggaatgct gtgtgtctgt ccggcacccc ggtaggcctg attgggtttc tagtcctcct |
| 2281 | taaccactta tctcccatat gagagtgtga aaaataggaa cacgtgctct acctccattt |
| 2341 | agggatttgc ttgggataca gaagaggcca tgtgtctcag agctgttaag ggcttatttt |
| 2401 | tttaaaacat tggagtcata gcatgtgtgt aaactttaaa tatgcaaata aataagtatc |
| 2461 | tatgtc |

As noted above, the polynucleotides of the disclosure may be DNA or RNA. In some embodiments, the polynucleotide is comprised in or on a vector. Suitable vectors for polynucleotide cancer cell delivery, in vivo or ex vivo, include any viral or non-viral vector. This includes, but is not limited to, lentivirus, vaccinia virus, adenovirus (replication competent, replication incompetent, helper dependent), adeno associated virus (AAV), Herpes simplex virus 1 (HSV1), myxoma virus, reovirus, poliovirus, vesicular stomatitis virus (VSV), measles virus (MV), Newcastle disease virus (NDV), retroviruses, nanoparticles, cationic lipids, cationic polymers, and/or lipid polymers, for example. The polynucleotide may be generated as part of the same molecule as a vector, the polynucleotide may be encompassed within a vector, and/or the polynucleotide may be attached to a vector.

Polynucleotides of the disclosure are non-natural polynucleotides that may be generated by any means, including, for example, by standard recombinant methods known in the art. Alternatively, the polynucleotides described herein can be synthetic polynucleotides, produced by in vitro chemical synthesis using phosphotriester, phosphite or phosphoramidite chemistry and solid phase techniques such as described in EP266032, or via deoxynucleoside H-phosphonate intermediates as described in U.S. Pat. No. 5,705,629, which are hereby incorporated by reference in its entirety.

The polynucleotides encoding the proteins of interest as described above may be combined, i.e., operatively linked or coupled to other nucleic acid sequences, including but not limited to, promoters, enhancers, polyadenylation signals, restriction enzyme sites, multiple cloning sites, coding segments, and the like, to create a suitable polynucleotide construct for cell delivery and expression.

In another embodiment, the agent that promotes microtubule turnover is a microtubule destabilizing agent. Suitable microtubule destabilizing agents that can be administered in conjunction with a KIF18A inhibitor in accordance with the methods described herein include, without limitation, nocodazole, vinca alkaloids (such as vincristine, vinblastine, vinorelbine, vindesine, vinflunine), colchicine, and Erubulin mesylate. Other suitable microtubule destabilizing agents include, without limitation, cryptophycins, combretastatin A-4-P, combretastatin A-1-P, ombrabulin, soblidotin, D24851, pseudolaric acid B, and embellistatin (see Fanale et al., “Stabilizing Versus Destabilizing the Microtubules: A Double-Edge Sword for an Effective Cancer Treatment Option?” Analytical Cellular Pathology 2015: 690916 (2015), which is hereby incorporated by reference in its entirety).

In some embodiments, the KIF18A inhibitor and agent that promotes microtubule turnover are administered concurrently. In some embodiments, KIF18A inhibitor and agent that promotes microtubule turnover are administered sequentially.

In some embodiments, the method of inhibiting proliferation of chromosome instable cancer cells and treating cancer further involves administering, in conjunction with the KIF18A inhibitor, a cyclin-dependent kinase (CDK) inhibitor to said subject or to the cancer cells. In some embodiments, the method involves administering the combination of a KIF18A inhibitor, an agent that promotes microtubule turnover, and a CDK inhibitor.

In accordance with this aspect of the disclosure, the CDK inhibitor is a CDK 4 and/or CDK6 inhibitor. Suitable CDK 4/6 inhibitors include, without limitation, CDK inhibitor is selected from 6-acetyl-8-cyclopentyl-5-methyl-2-[(5-piperazin-1-ylpyridin-2-yl)amino]pyrido[6,5-d]pyrimidin-7-one (palbociclib), 7-cyclopentyl-N,N-dimethyl-2-{[5-(piperazin-1-yl)pyridin-2-yl]amino}-7H-pyrrolo[2,3-d]pyrimidine-6-carboxamide (ribociclib), N-[5-[(4-ethylpiperazin-1-yl)methyl]pyridin-2-yl]-5-fluoro-4-(7-fluoro-2-methyl-3-propan-2-ylbenzimidazol-5-yl)pyrimidin-2-amine (abemaciclib), 2-[[5-(4-methylpiperazin-1-yl)pyridin-2-yl ]amino]spiro[7,8-dihydropyrazion[5,6]pyrrolo[1,2-d]pyrimidine-9,1′-cyclohexane]-6-one (trilaciclib), 6-(difluoromethyl)-8-[(1R,2R)-2-hydroxy-2-methylcyclopentyl]-2-[(1-methylsulfonylpiperidin-4-yl)amino]pyrido[2,3-d]pyrimidin-7-one (PF-06873600), and MMD37k (a synthetic peptide inhibitor of CDK4/6). In some embodiments, the CDK inhibitor is selected from palbociclib, ribociclib, and abemaciclib.

In some embodiments, the KIF18A inhibitor, the agent that promotes microtubule turnover, and/or the CDK inhibitor are administered concurrently. In some embodiments, the KIF18A inhibitor, the agent that promotes microtubule turnover, and/or the CDK inhibitor are administered sequentially.

In some embodiments, the combination therapy as described herein, e.g., the KIF18A inhibitor together with the agent that promotes microtubule turnover and/or the CDK inhibitor, provides a synergistic effect, as measured by, for example, the extent of cancer cell proliferation, the response rate, the time to disease progression, or the survival period, as compared to the effect achievable on dosing with the KIF18A alone. For example, the effect of the combination treatment is defined as affording a synergistic effect if the KIF18A inhibitor is administered at a dose lower than its dose when administered alone and the therapeutic effect, as measured by, for example, the extent of inhibiting cancer cell proliferation, the response rate, the time to disease progression or the survival period, is equivalent to that achievable on dosing higher amounts of KIF18A inhibitor alone In particular, synergy is deemed to be present if the dose of the KIF18A inhibitor is reduced without detriment to one or more of the extent of the response, the response rate, the time to disease progression, and survival data, in particular without detriment to the duration of the response, but with fewer and/or less troublesome side-effects than those that occur when conventional doses of each component are used. In some embodiments, the effect of the combination treatment is defined as affording a synergistic effect if the agent that promotes microtubule turnover and/or the CDK inhibitor is administered at a dose lower than when administered alone, and the therapeutic effect, as measured by, for example, the extent cancer cell proliferation, the response rate, the time to disease progression or the survival period, is equivalent to that achievable on dosing higher amounts of the agent that promotes microtubule turnover and/or CDK inhibitor alone. In particular, synergy is deemed to be present if the dose of the agent that promotes microtubule turnover and/or CDK inhibitor is reduced without detriment to one or more of the extent of the response, the response rate, the time to disease progression, and survival data, in particular without detriment to the duration of the response, but with fewer and/or less troublesome side-effects than those that occur when conventional doses of each component are used.

In accordance with the methods described herein, administration of the KIF18A inhibitor alone or in combination with an agent that promotes microtubule turnover and/or CDK inhibitor as described herein is carried out by systemic or local administration. Suitable modes of systemic administration of the therapeutic agents and/or combination therapeutics disclosed herein include, without limitation, orally, topically, transdermally, parenterally, intradermally, intrapulmonary, intramuscularly, intraperitoneally, intravenously, subcutaneously, or by intranasal instillation, by intracavitary or intravesical instillation, intraocularly, intra-arterially, intralesionally, or by application to mucous membranes. In certain embodiments, the therapeutic agents of the methods described herein are delivered orally. Suitable modes of local administration of the KIF18A inhibitor alone or in combination with an agent that promotes microtubule turnover and/or CDK inhibitor as disclosed herein include, without limitation, catheterization, implantation, direct injection, dermal/transdermal application, or portal vein administration to relevant tissues, or by any other local administration technique, method or procedure generally known in the art. The mode of affecting delivery of agent will vary depending on the type of therapeutic agent and the type of cancer to be treated.

A therapeutically effective amount of the KIF18A inhibitor alone or in combination with the agent that promotes microtubule turnover and/or CDK inhibitor in the methods disclosed herein is an amount that, when administered over a particular time interval, results in achievement of one or more therapeutic benchmarks (e.g., slowing or halting of cancer cell proliferation, slowing or halting of cancer growth, cancer regression, cessation of symptoms, etc.). The KIF18A inhibitor alone or in combination with an agent that promotes microtubule turnover and/or CDK inhibitor for use in the presently disclosed methods may be administered to a subject one time or multiple times. In those embodiments where the compounds are administered multiple times, they may be administered at a set interval, e.g., daily, every other day, weekly, or monthly. Alternatively, they can be administered at an irregular interval, for example on an as-needed basis based on symptoms, patient health, and the like. For example, a therapeutically effective amount may be administered once a day (q.d.) for one day, at least 2 days, at least 3 days, at least 4 days, at least 5 days, at least 6 days, at least 7 days, at least 10 days, or at least 15 days. Optionally, the status of the cancer or the regression of the cancer is monitored during or after the treatment, for example, by a multiparametric ultrasound (mpUS), multiparametric magnetic resonance imaging (mpMRI), and nuclear imaging (positron emission tomography [PET]) of the subject. The dosage of the KIF18A inhibitor or combination therapy administered to the subject can be increased or decreased depending on the status of the cancer or the regression of the cancer detected.

The skilled artisan can readily determine this amount, on either an individual subject basis (e.g., the amount of a compound necessary to achieve a particular therapeutic benchmark in the subject being treated) or a population basis (e.g., the amount of a compound necessary to achieve a particular therapeutic benchmark in the average subject from a given population). Ideally, the therapeutically effective amount does not exceed the maximum tolerated dosage at which 50% or more of treated subjects experience side effects that prevent further drug administrations.

A therapeutically effective amount may vary for a subject depending on a variety of factors, including variety and extent of the symptoms, sex, age, body weight, or general health of the subject, administration mode and salt or solvate type, variation in susceptibility to the drug, the specific type of the disease, and the like.

The effectiveness of the methods of the present application in inhibiting cancer cell proliferation and/or treating cancer may be evaluated, for example, by assessing changes in tumor burden and/or disease progression following treatment with the KIF18A inhibitor alone or in combination with an agent that promotes microtubule turnover and/or CDK inhibitor as described herein according to the Response Evaluation Criteria in Solid Tumours (Eisenhauer et al., “New Response Evaluation Criteria in Solid Tumours: Revised RECIST Guideline (Version 1.1),” Eur. J. Cancer 45(2): 228-247 (2009), which is hereby incorporated by reference in its entirety). In some embodiments, tumor burden and/or disease progression is evaluated using imaging techniques including, e.g., X-ray, computed tomography (CT) scan, magnetic resonance imaging, multiparametric ultrasound (mpUS), multiparametric magnetic resonance imaging (mpMRI), and nuclear imaging (positron emission tomography [PET]) (Eisenhauer et al., “New Response Evaluation Criteria in Solid Tumours: Revised RECIST Guideline (Version 1.1),” Eur. J. Cancer 45(2): 228-247 (2009), which is hereby incorporated by reference in its entirety). Cancer regression or progression may be monitored prior to, during, and/or following treatment with one or more of the therapeutic agents described herein.

In some embodiments, the response to treatment with the methods described herein results in at least about 1%, 2%, 3%, 4%, 5%, 6%, 7%, 8%, 9%, 10%, 20%, 30%, 40%, 50%, 60%, 70%, 80%, 90%, 95%, 99%, or 100% decrease in tumor size as compared to baseline tumor size. Thus, the response to treatment with any of the methods described herein may be partial (e.g., at least a 30% decrease in tumor size, as compared to baseline tumor size) or complete (elimination of the tumor).

In some embodiments, the methods described herein may be effective to inhibit cancer cell proliferation, inhibit cancer growth, inhibit cancer progression, reduce primary tumor size, relieve tumor-related symptoms, inhibit tumor-secreted factors (e.g., tumor-secreted hormones), delay the appearance of primary or secondary cancer tumors, slow development of primary or secondary cancer tumors, decrease the occurrence of primary or secondary cancer tumors, slow or decrease the severity of secondary effects of disease, arrest tumor growth, and/or achieve regression of cancer in a selected subject.

In some embodiments, the methods described herein reduce the rate of tumor growth in the selected subject by at least about 1%, 2%, 3%, 4%, 5%, 6%, 7%, 8%, 9%, 10%, 20%, 30%, 40%, 50%, 60%, 70%, 80%, 90%, 95%, 99%, or more. In certain embodiments, the methods described herein reduce the rate of tumor invasiveness in the selected subject by at least about 1%, 2%, 3%, 4%, 5%, 6%, 7%, 8%, 9%, 10%, 20%, 30%, 40%, 50%, 60%, 70%, 80%, 90%, 95%, 99%, or more. In specific embodiments, the methods described herein reduce the rate of tumor progression in the selected subject by at least about 1%, 2%, 3%, 4%, 5%, 6%, 7%, 8%, 9%, 10%, 20%, 30%, 40%, 50%, 60%, 70%, 80%, 90%, 95%, 99%, or more.

Another aspect of the disclosure relates to a combination therapeutic. As used herein, the term “combination therapeutic” refers to two or more therapeutic agents, i.e., a KIF18A inhibitor in combination with an agent that promotes microtubule turnover and/or a CDK inhibitor, suitable for the treatment of a cancer exhibiting chromosomal instability. In some embodiments, the combination therapy is formulated for co-administeration in a substantially simultaneous manner, such as in a single capsule or other delivery vehicle having a fixed ratio of active ingredients. In some embodiments, the combination therapy is formulated for administration in multiple capsules or delivery vehicles, each containing an active ingredient. In some embodiments, the therapeutic agents of the combination therapy are administered in a sequential manner, either at approximately the same time or at different times. For example, in one embodiment, the KIF18A is administered prior to the administration of the agent that promotes microtubule turnover and/or a CDK inhibitor. In other embodiments, the KIF18A inhibitor is administered simultaneously with the agent that promotes microtubule turnover and/or a CDK inhibitor. In all embodiments, the combination therapy provides beneficial effects of the drug combination in treating chromosomal instable cancer.

In some embodiments, the combination therapeutic comprises an inhibitor of KIF18A and an agent that promotes microtubule turnover. Suitable KIF18A inhibitors and agents that promote kinetochore turnover, e.g., a MCAK activating agent, are described supra. In some embodiments, the combination therapeutic comprises a KIF18A inhibitor and a MCAK activation agent (e.g., UMK57).

In some embodiments, the combination therapeutic comprises a KIF18A inhibitor and a CDK inhibitor. In some embodiments, the CDK inhibitor is a CDK 4/6 inhibitor. Suitable CDK4/6 inhibitors are disclosed supra. In some embodiments, the combination therapeutic comprises a KIF18A inhibitor, an agent that promotes microtubule turnover, and a CDK inhibitor.

The therapeutic agents and combination therapeutics described herein can be formulated into pharmaceutical compositions as any one or more of the active compounds described herein and a physiologically acceptable carrier (also referred to as a pharmaceutically acceptable carrier or solution or diluent). Such carriers and solutions include pharmaceutically acceptable salts and solvates of compounds used in the methods described herein, and mixtures comprising two or more of such compounds, pharmaceutically acceptable salts of the compounds and pharmaceutically acceptable solvates of the compounds. Such compositions are prepared in accordance with acceptable pharmaceutical procedures such as described in Remington: The Science and Practice of Pharmacy, 20th edition, ed. Alfonso R. Gennaro (2000), which is incorporated herein by reference in its entirety.

The term “pharmaceutically acceptable carrier” refers to a carrier that does not cause an allergic reaction or other untoward effect in patients to whom it is administered and are compatible with the other ingredients in the formulation. Pharmaceutically acceptable carriers include, for example, pharmaceutical diluents, excipients or carriers suitably selected with respect to the intended form of administration, and consistent with conventional pharmaceutical practices. For example, solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof. Pharmaceutically acceptable carriers may further comprise minor amounts of auxiliary substances such as wetting or emulsifying agents, preservatives or buffers, which enhance the shelf life or effectiveness of the therapeutic agent.

Preferences and options for a given aspect, feature, embodiment, or parameter of the technology described herein should, unless the context indicates otherwise, be regarded as having been disclosed in combination with any and all preferences and options for all other aspects, features, embodiments, and parameters of the technology.

The present technology may be further illustrated by reference to the following examples.

### EXAMPLES

The examples below are intended to exemplify the practice of embodiments of the disclosure but are by no means intended to limit the scope thereof.

### Example 1—Cell Culture and Transfections

HT29, LoVo, SW480, LS1034, HCC1806, HCT116, MCF10A, MDA-MB-231, and MDA-MB-468 cells were purchased from ATCC (Manassas, Va.). All cell lines were validated by STR DNA fingerprinting using the Promega GenePrint® 10 System according to manufacturer's instructions (Promega #B9510). HT29, LoVo, SW480, MDA-MB-231, and MDA-MB-468 cells were cultured in DMEM/F-12 medium (Gibco) supplemented with 10% FBS (Gibco) and 1% penicillin/streptomycin (pen/strep). LS1034 and HCC1806 cells were cultured in RPMI 1640 medium (Gibco) with 10% FBS and 1% pen/strep. HCT116 cells were cultured in McCoys 5A media (Gibco) with 10% FBS and 1% pen/strep, and MCF10A cells were cultured in DMEM/F-12 supplemented with 5% horse serum (Gibco), 20 ng/ml epidermal growth factor, 0.5 μg/ml hydrocortisone, 100 ng/ml cholera toxin, 10 μg/ml insulin, and 1% pen/strep. To inhibit specific kinesins, cells were treated with 5 pmol siRNA with Lipofectamine RNAiMAX Transfection Reagent (Invitrogen, Carlsbad, Calif.) in Opti-MEM Reduced-Serum Media (Gibco). Specific siRNAs include pools of Silencer and Silencer Select KIF18A (Invitrogen, Carlsbad, Calif.), KIF18B (Dharmacon), KIF4A (Invitrogen, Carlsbad, Calif.), KID/KIF22 (Invitrogen, Carlsbad, Calif.), MCAK/KIFf2C (Dharmacon, Lafayette, Colo.), MAD2 (Invitrogen, Carlsbad, Calif.), and pools of scrambled-sequence negative control siRNAs (Invitrogen, Carlsbad, Calif.). For double knockdowns involving the inhibition of two proteins, Lipofectamine RNAiMAX was used at a lowered concentration (0.7× the concentration used for single knockdowns) to mitigate toxicity.

### Example 2—Drug Treatments

For experiments involving siRNA knockdown followed by drug treatment, the indicated concentrations of paclitaxel (Selleck Chemicals, Houston, Tex.), nocodazole (Selleck Chemicals, Houston, Tex.), and/or monastrol (Selleck Chemicals, Houston, Tex.) were added to cells 24 h after siRNA treatment. Three hours after drug addition, cells were either fixed and stained for immunofluorescence imaging or imaged live in a glass-bottom 24-well dish. To compare the effects of paclitaxel treatment to the effects of KIF18A KD in MDA-MB-231 and MCF10A cell lines, 10 nM of paclitaxel was added to cells 24 h before fixing and staining for immunofluorescence imaging.

### Example 3—Proliferation and Cytotoxicity Assays

Cells were imaged in either a 96- or 24-well dish every two or four hours for up to five days using the Cytation 5 Cell Imaging Multi-Mode Reader (Biotek, Winooski, Vt.) driven by Gen5 software (Biotek, Winooski, Vt.). A 4×Plan Fluorite 0.13 NA objective (Olympus) was used to capture images. Between imaging reads, cells were incubated at 37° C. with 5% CO₂ using the Biospa 8 Automated Incubator (Biotek, Winooski, Vt.). Gen5 software (Biotek, Winooski, Vt.) was used to process images and to measure cell confluence and the number of cells/mm² using high-contrast brightfield images. Parameters including cell size and light-intensity thresholds were specified for each cell line. To determine rates of cell proliferation, the fold change in cells/mm² between the first and last reads of each well were calculated and normalized to the control for each experiment. One-way ANOVA with post-hoc Tukey's test was used to compare proliferation fold-change values across cell lines to determine statistical significance. For cytotoxicity assays, CellTox™ Green Dye (Promega, Madison, Wis.) was added to cell media prior to imaging, and the number of cells/mm² was recorded for both GFP and brightfield channels. After four days of imaging, the area under the proliferation curve for the CellTox-stained cells was divided by the area under the proliferation curve for the total number of cells, and this value was normalized to the control for each cell line as the metric for relative cell death. An unpaired t-test was used to determine significance between control and KIF18A KD for each cell line.

### Example 4—Automated Cell Count Validation

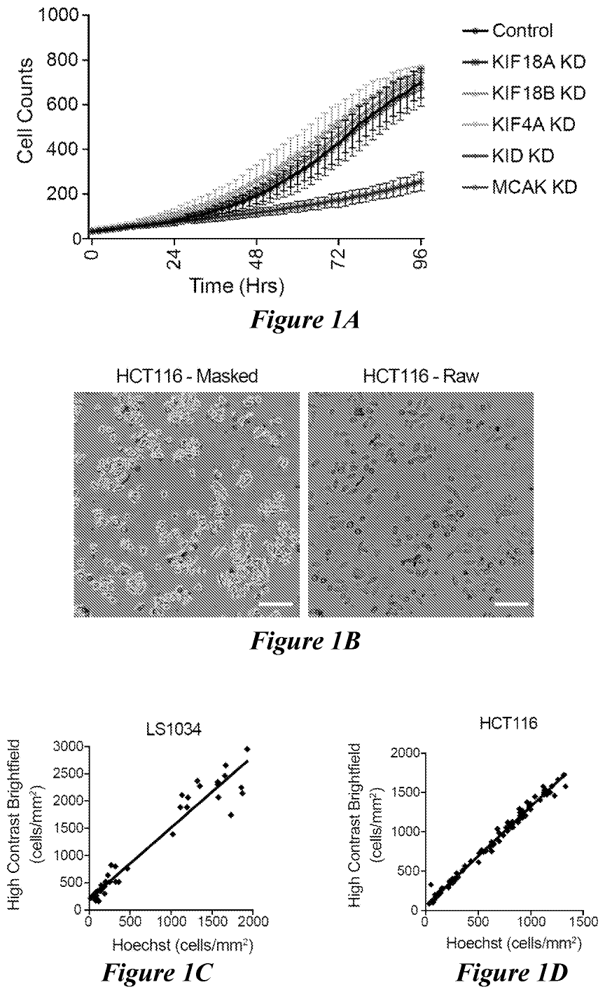

Cells were seeded in a series of increasing densities in either a 96- or 24-well dish and allowed to adhere for 24 hours. Cells were then incubated with Hoechst stain (Invitrogen, Carlsbad, Calif.), a cell-permeable nuclear dye, for 30 minutes before being imaged using the Cytation 5 system as described previously. For each field, one high-contrast brightfield image and one fluorescence image were acquired, and Gen5 software was used to process images and analyze the number of cells/mm² using the parameters defined in the proliferation assays. The correlation between cell densities measured in the brightfield images and the fluorescence images was graphed as a scatterplot (FIGS. 1A-1D).

### Example 5—Immunofluorescence

Cells were grown on glass coverslips and fixed using either −20° C. methanol or 1% paraformaldehyde in −20° C. methanol. Cells were blocked with 20% goat serum in antibody diluting buffer (Abdil—TBS, 1% BSA, 0.1% Triton X-100, and 0.1% sodium azide) and incubated with the following primary antibodies: mouse anti-α-tubulin (DM1α) 1:500 (Millipore Sigma, Burlington Mass.) for one hour at room temperature (RT), human anti-centromere antibody (ACA) 1:250 (Antibodies Incorporated) overnight at 4° C., rabbit anti-γ-tubulin 1:500 (Abcam, Cambridge, Mass.) for one hour at RT, mouse anti-γ-tubulin 1:500 for one hour at RT (Abcam, Cambridge, Mass.), rabbit anti-KIF18A 1:100 (Bethyl Laboratories, Montgomery, Tex.) at 4° C. overnight, mouse anti-centrin-1 1:500 (Santa Cruz Biotechnology) for one hour at RT, and rabbit KIF18B 1:2000 (Shin et al., “Biased Brownian Motion as a Mechanism to Facilitate Nanometer-scale Exploration of the Microtubule Plus End by a Kinesin-8,” Proc National Acad Sci 112:E3826-35 (2015), which is hereby incorporated by reference in its entirety) for one hour at RT. Secondary antibodies conjugated to Alexa Fluor 488, 594, and 647 (Molecular Probes, Eugene, Oreg.) were used at concentrations of 1:15000 for one hour at RT. Coverslips were mounted onto glass slides using Prolong Gold anti-fade mounting medium with DAPI (Molecular Probes, Eugene, Oreg.).

### Example 6—Microscopy

Fixed and live cell images were acquired using a Ti-E or Ti-2E inverted microscope (Nikon Instruments) driven by NIS Elements software (Nikon Instruments). Images were captured using a Clara cooled charge-coupled device (CCD) camera (Andor, Concord, Mass.) or Prime Bsi sCMOS camera (Teledyne Photometrics, Tucson, Ariz.) with a Spectra-X light engine (Lumencore, Beaverton, Oreg.). For live-cell imaging, cells in CO₂-independent media (Gibco) were imaged using Nikon objectives Plan Apo 20×0.75 NA or 40×0.95 NA and an environmental chamber at 37° C. Fixed cell images were taken using Plan Apo 40×0.95 NA, Plan Apo λ 60×1.42 NA, and APO 100×1.49 NA (Nikon).

### Example 7—Western Blot

Cells were lysed in PHEM lysis buffer (60 mM Pipes, 10 mM EGTA, 4 mM MgCl₂, and 25 mM Hepes) with 1% Triton X-100 and protease inhibitors, incubated on ice for 10 minutes, and centrifuged at maximum speed for 5 minutes. Laemmli buffer with β-mercaptoethanol was added to the supernatant prior to boiling for 10 minutes at 95° C. Lysates were run on 4-15% gradient gels (BioRad, Hercules, Calif.), transferred (75 minutes at 100V) to PVDF membrane (BioRad, Hercules, Calif.), and blocked for one hour in 1:1 Odyssey Blocking Buffer (Li-Cor, Lincoln, Nebr.) and TBS with 0.1% Tween-20. Membranes were incubated with primary antibodies overnight at 4° C. Primary antibodies included 1:1000 mouse anti-GAPDH (Invitrogen, Carlsbad, Calif.), 1:500 rabbit anti-KIF18A (Bethyl Laboratories, Montgomery, Tex.), 1:1000 rabbit anti-Kif4A (Bethyl Laboratories), 1:1000 rabbit anti-KIF22 (Millipore Sigma, Burlington, Mass.), 1:1000 rabbit anti-MCAK (Abcam), 1:1000 rabbit anti-MAD2 (Bethyl Laboratories, Montgomery, Tex.), and 1:1000 rabbit anti-Cleaved Caspase-3 (Cell Signaling Technology, Danvers, Mass.). Secondary antibodies included goat anti-Rabbit IgG DyLight 800 conjugate and goat anti-mouse IgG DyLight 680 (Invitrogen, Carlsbad, Calif.), which were each diluted to 1:15000 in 1:1 Odyssey blocking buffer/TBS and added to the membrane for one hour at room temperature. Membranes were imaged using an Odyssey CLx (Li-Cor, Lincoln, Nebr.).

### Example 8—Live Imaging with Sir-Tubulin

Cells were plated in a glass-bottom 24-well dish and treated with the indicated siRNA approximately 24 hours before imaging. Six hours before imaging, the cell culture media was replaced with CO₂-independent media containing 100 μM SiR-tubulin (Cytoskeleton). For conditions involving UMK57 or DMSO, the specified drug was added to the CO₂-independent media with siR-tubulin. Cells were imaged every 2 minutes for 16-20 hours using a 40×0.75 NA objective (Nikon).

### Example 9—Mitotic Timing and Mitotic Index Analyses

To measure the length of mitosis, live cells were imaged every two minutes for 16-20 hours using differential interference contrast (DIC) microscopy. The time between nuclear envelope breakdown (NEB) and anaphase onset (AO) was used to indicate the time a cell spent in mitosis. Mitotic index was measured using fixed-cell images by counting the number of mitotic cells divided by the total number of cells. All mitotic index fields were taken with a 40×objective. An unpaired t-test was used to determine statistical significance between control and KIF18A KD conditions for each cell line.

### Example 10—Mitotic Spindle Morphology Analyses

To analyze mitotic spindle morphology, cells were fixed and stained for γ-tubulin, α-tubulin, and centrin-1. Enough optical slices spaced 200 nm apart were captured to visualize the entire 3-D structure of the spindle. Spindles with three or more visible microtubule-organizing centers were classified as multipolar. Cells were considered to have fragmented pericentriolar material (PCM) if they had supernumerary poles observed via γ-tubulin staining but lacked centrioles (centrin-1 puncta) at one or more of the poles. Intercentriolar distance, or the distance in microns between two centrioles in a pair, was measured from the center of one centriole to the center of the adjacent centriole.

### Example 11—Knockdown Quantification Analysis

The efficiency of siRNA-mediated kinesin knockdowns was measured via either quantitative western blot or immunofluorescence. ImageJ was used for all quantification. KIF18A knockdown efficiency in CRC cell lines was measured by comparing background-subtracted KIF18A fluorescence intensity in cells treated with control or KIF18A siRNA. In TNBC cell lines, KIF18B knockdown efficiency was measured by comparing background-subtracted KIF18B fluorescence intensity in cells treated with control or KIF18B siRNA. All other knockdown quantifications were determined by Western blot analysis. For MCF10A and MDA-MB-231 cell lines, the KIF18A knockdown efficiency was further analyzed at the RNA level by qRT-PCR.

### Example 12—QRT-PCR

Total RNA extraction was carried out using RNeasy Mini Kit (Qiagen, Dusseldorf, Germany). Extracted RNA was screened by the Vermont Integrative Genomics Resource (VIGR) DNA Facility for purity and integrity using a 2100 Bioanalyzer (Agilent Technologies, Santa Clara, Calif.), and human GAPDH and human KIF18A Taqman probes and primers (Thermo Fisher Scientific, Waltham, Mass.) were used for reverse transcription and qRT-PCR. KIF18A RNA expression levels were normalized to GAPDH RNA levels in each cell line.

### Discussion of Examples 1-12

In the present disclosure the inventors tested the hypothesis that altered mitotic microtubule dynamics in CIN cells may confer sensitivity to inhibition of proteins that regulate microtubule dynamics or generate forces within mitotic spindles. Ideal targets would reduce CIN cell proliferation by inducing mitotic defects specifically in tumor cells. Efforts were focused on kinesin motors known to regulate spindle microtubule dynamics and mechanics that are also largely dispensable for division in diploid somatic cells

KIF18A is Required for the Proliferation of CIN Tumor Cells but not Diploid Cells

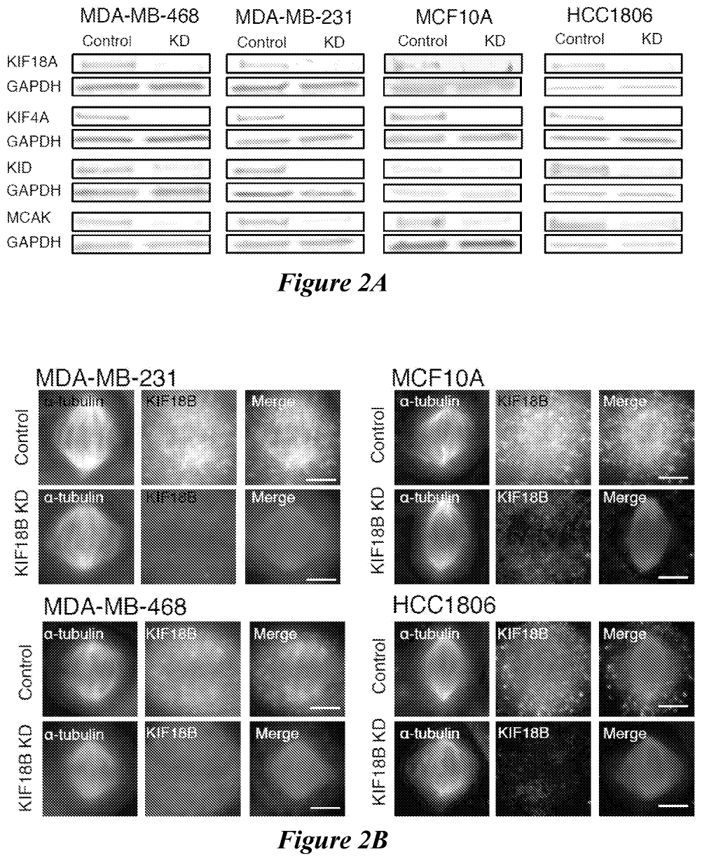

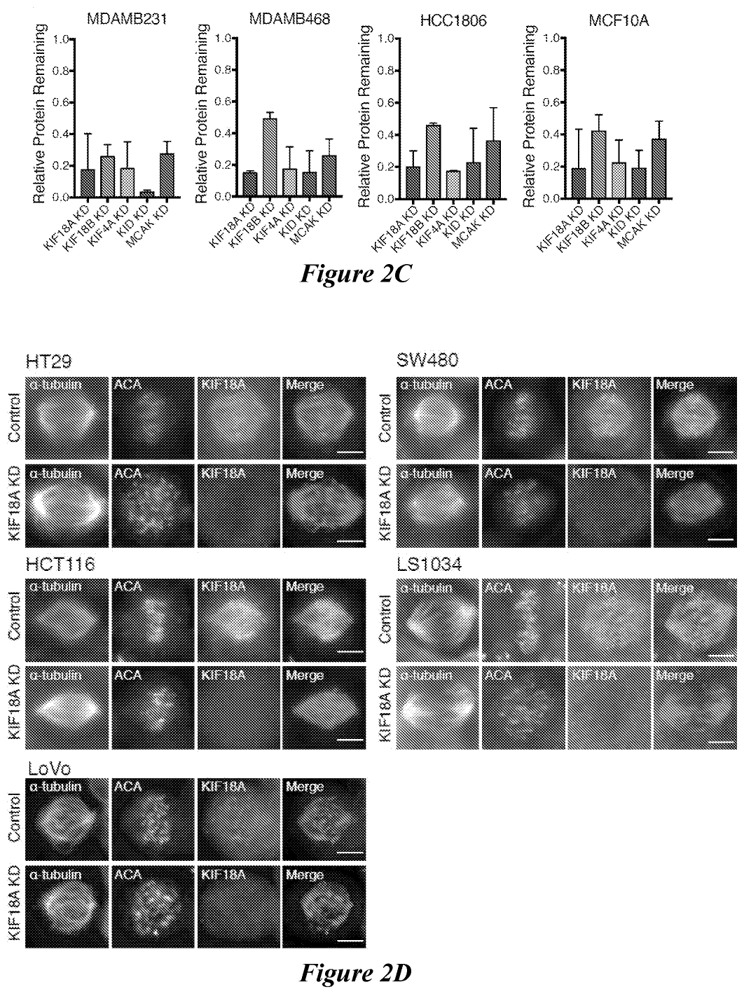

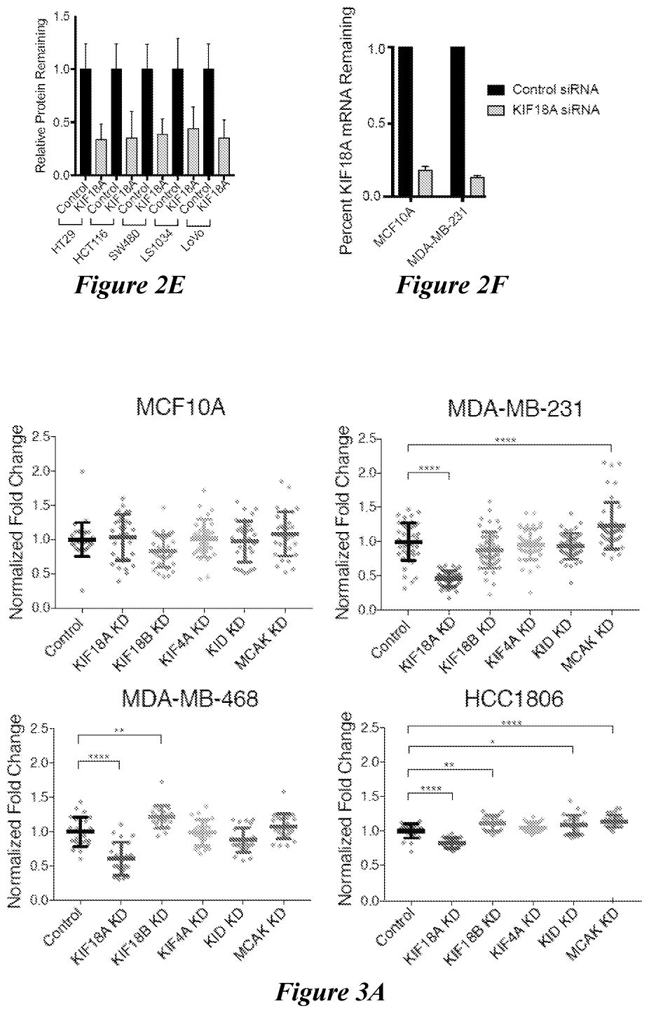

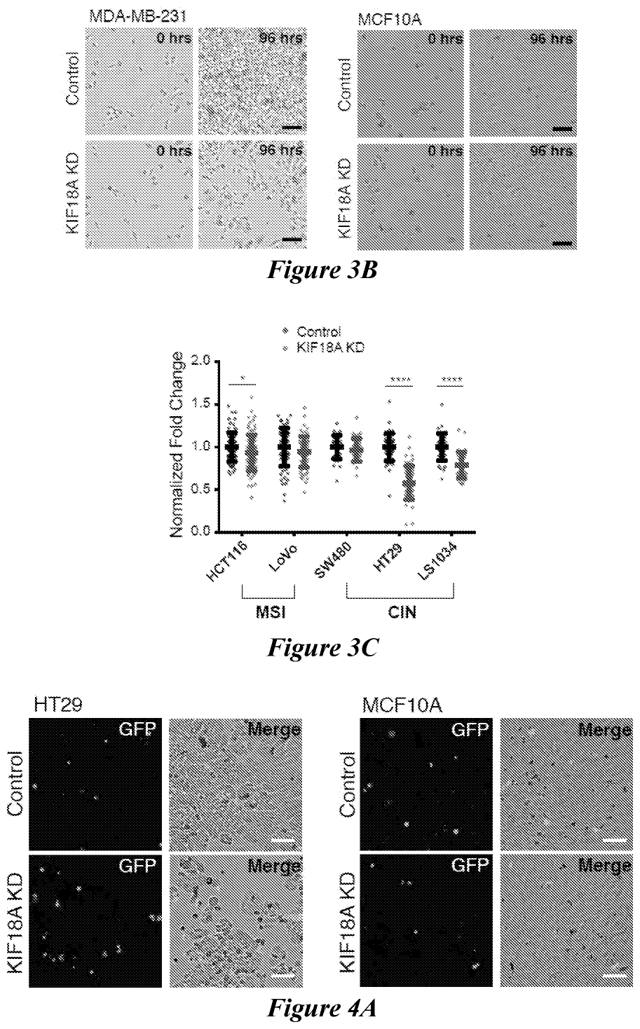

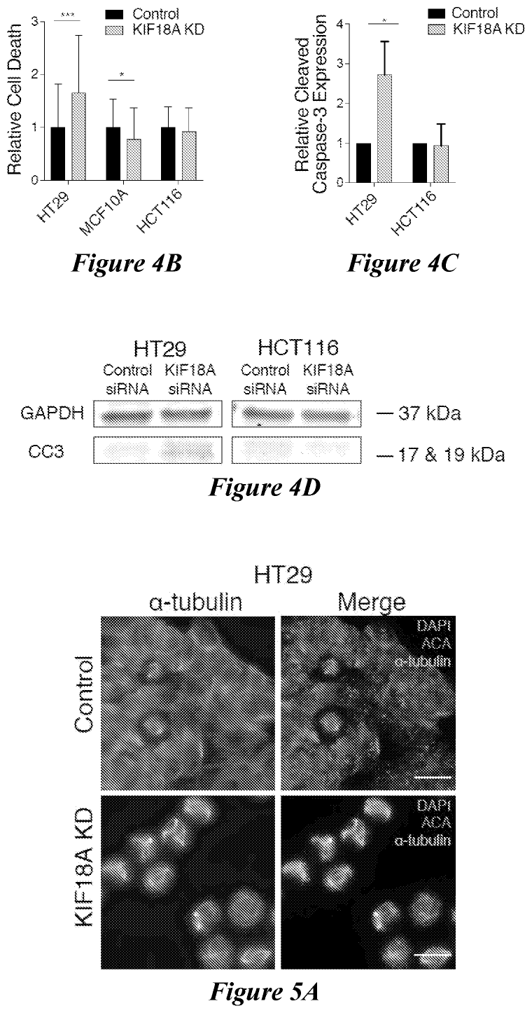

To compare the impacts of altered kinesin function in cells with or without CIN, cell proliferation in was measured both stable, diploid breast epithelial MCF10A cells and the chromosomally unstable triple negative breast cancer (TNBC) cell lines MDA-MB-231, MDA-MB-468, and HCC1806 (Lehmann et al., “Identification of Human Triple-negative Breast Cancer Subtypes and Preclinical Models for Selection of Targeted Therapies,” J Clin Invest 121:2750-2767 (2011), which is hereby incorporated by reference in its entirety) following knockdown (KD) of kinesin motor proteins. Specifically, the effects of KIF18A, KIF18B, KIF4A, KIF22/KID, and KIF2C/MCAK KD were determined (FIGS. 2A-2F). Cell proliferation was measured using an automated high-contrast brightfield microscopy-based kinetic assay (FIGS. 1A-1D). KIF18A KD significantly reduced proliferation of all three TNBC cell lines, but did not affect the growth of diploid MCF10A cells (FIGS. 3A-3B). To determine if this trend holds in other tumor cell types, proliferation in colorectal cancer (CRC) cells categorized as displaying either chromosomal instability (CIN) or microsatellite instability (MSI), a form of genomic instability arising from defective DNA repair in near-diploid tumor cells (Mouradov et al., “Colorectal Cancer Cell Lines Are Representative Models of the Main Molecular Subtypes of Primary Cancer,” Cancer Res 74:3238-47 (2014), which is hereby incorporated by reference in its entirety) were measured. KIF18A KD significantly reduced the proliferation of two CIN cell lines but had minor effects on the proliferation of MSI cells (FIG. 3C, FIGS. 2A-2F). CIN cells also exhibited increased cell death following KIF18A KD, while near-diploid HCT116 and MCF10A cells did not (FIGS. 4A-4B). These data indicate that, while diploid cells do not require KIF18A to proliferate, a subset of CIN tumor cells are dependent on KIF18A for efficient growth and survival.

Loss of KIF18A Induces Prolonged Mitotic Delay in CIN Tumor Cells

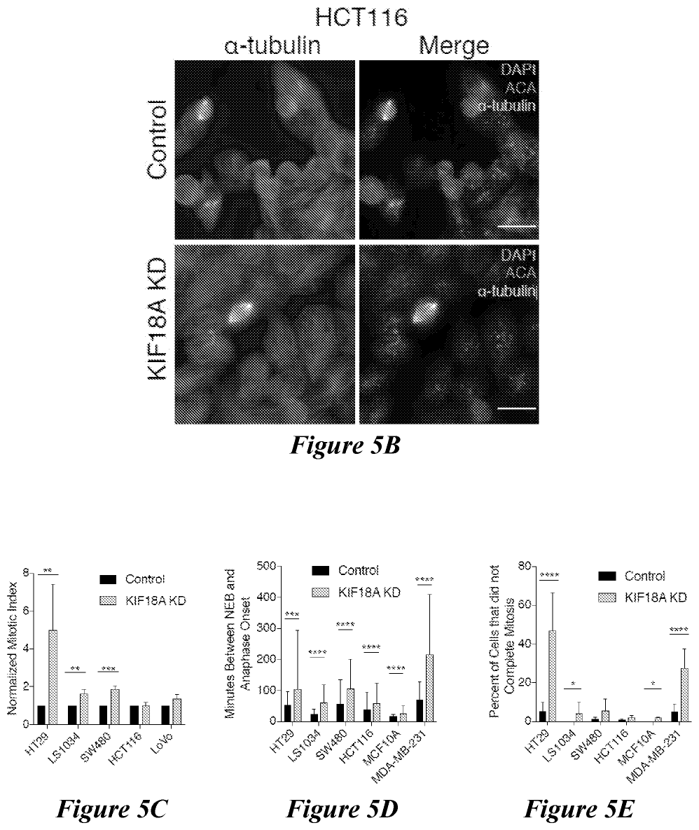

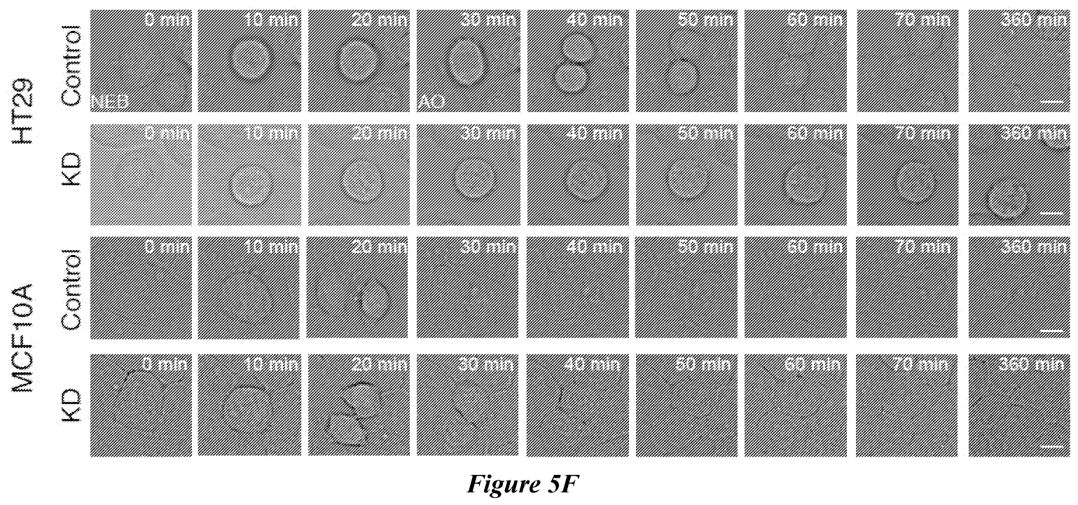

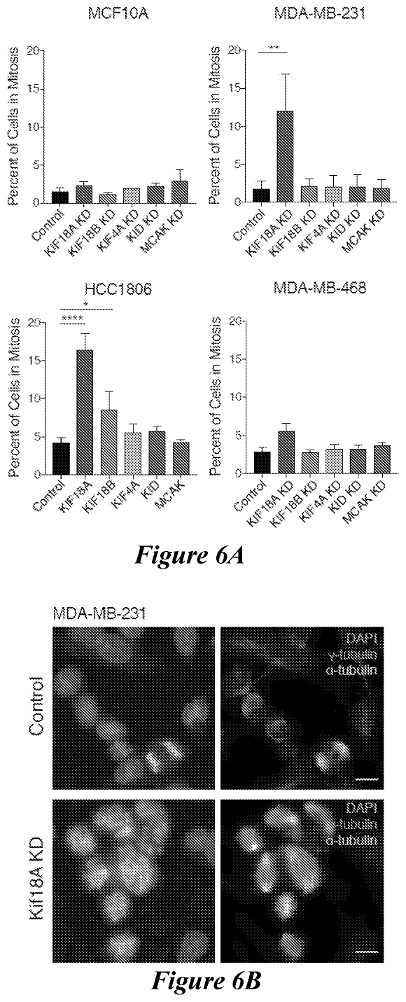

KIF18A is required for chromosome alignment in all cells but also promotes spindle assembly checkpoint satisfaction and progression through mitosis in some cell types (Mayr et al., “The Human Kinesin Kif18A is a Motile Microtubule Depolymerase Essential for Chromosome Congression,” Curr Biol 17:488-498 (2007); Stumpff et al., “The Kinesin-8 Motor Kif18A Suppresses Kinetochore Movements to Control Mitotic Chromosome Alignment,” Dev Cell 14:252-262 (2008); Czechanski et al., “Kif18a is Specifically Required for Mitotic Progression During Germ Line Development,” Dev Biol 402:253-262 (2015); Fonseca et al., “Mitotic Chromosome Alignment Ensures Mitotic Fidelity by Promoting Interchromosomal Compaction During Anaphase,” J Cell Biol 218:1086-1088 (2019); Janssen et al., “Loss of Kif18A Results in Spindle Assembly Checkpoint Activation at Microtubule-Attached Kinetochores,” Curr Biol 28(17):2685-2696 (2018); Edzuka & Goshima, “Drosophila Kinesin-8 Stabilizes the Kinetochore-microtubule Interaction,” J Cell Biol 5:jcb.201807077 (2018); Zhu et al., “Functional Analysis of Human Microtubule-based Motor Proteins, the Kinesins and Dyneins, in Mitosis/Cytokinesis Using RNA Interference,” Mol Biol Cell 16:3187-3199 (2005), which are hereby incorporated by reference in their entirety). To determine if proliferation defects seen in KIF18A-depleted CIN cells are due to KIF18A's role in promoting timely metaphase-to-anaphase transitions, the effects of KIF18A KD on mitotic progression in CIN cells and near-diploid cells were compared. KIF18A KD led to an increase in the percentage of mitotic CIN cells but did not significantly alter the percentage of mitotic cells within MCF10A or MSI CRC cell populations (FIGS. 5A-5C and FIGS. 6A-6B). Quantification of mitotic duration revealed that all cell types displayed a significant increase in the amount of time required to progress from nuclear envelope breakdown (NEB) to anaphase onset (AO) following KIF18A KD (FIGS. 5D-5F). Consistent with previous work, the magnitude and variance of mitotic delays were larger in KIF18A KD CIN tumor cells than diploid (MCF10A) or near-diploid cells (HCT116) (FIG. 5D) (Czechanski et al., “Kif18a is Specifically Required for Mitotic Progression During Germ Line Development,” Dev Biol 402:253-262 (2015); Fonseca et al., “Mitotic Chromosome Alignment Ensures Mitotic Fidelity by Promoting Interchromosomal Compaction During Anaphase,” J Cell Biol 218:1086-1088 (2019); Janssen et al., “Loss of Kif18A Results in Spindle Assembly Checkpoint Activation at Microtubule-Attached Kinetochores,” Curr Biol 28(17):2685-2696 (2018); Hafner et al., “Pre-anaphase Chromosome Oscillations Are Regulated by the Antagonistic Activities of Cdk1 and PP1 on Kif18A,” Nat Commun 5:4397 (2014); Malaby et al., “KIF18A's Neck Linker Permits Navigation of Microtubule-bound Obstacles Within the Mitotic Spindle,” Life Sci Alliance 2:e201800169 (2019), which are hereby incorporated by reference in their entirety). In addition, the cell types most sensitive to KIF18A KD contained a significant subpopulation of cells that failed to complete mitosis during the imaging studies and were arrested for up to 20 hours (FIG. 5E). Interestingly, SW480 CIN cells did not display an increase in mitotically arrested cells and were also not dependent on KIF18A for proliferation. These data suggest that proliferation defects in KIF18A-dependent CIN cells may stem from defects that prevent subpopulations of cells from completing mitosis.

KIF18A-Dependent CIN Cells Form Multipolar Spindles

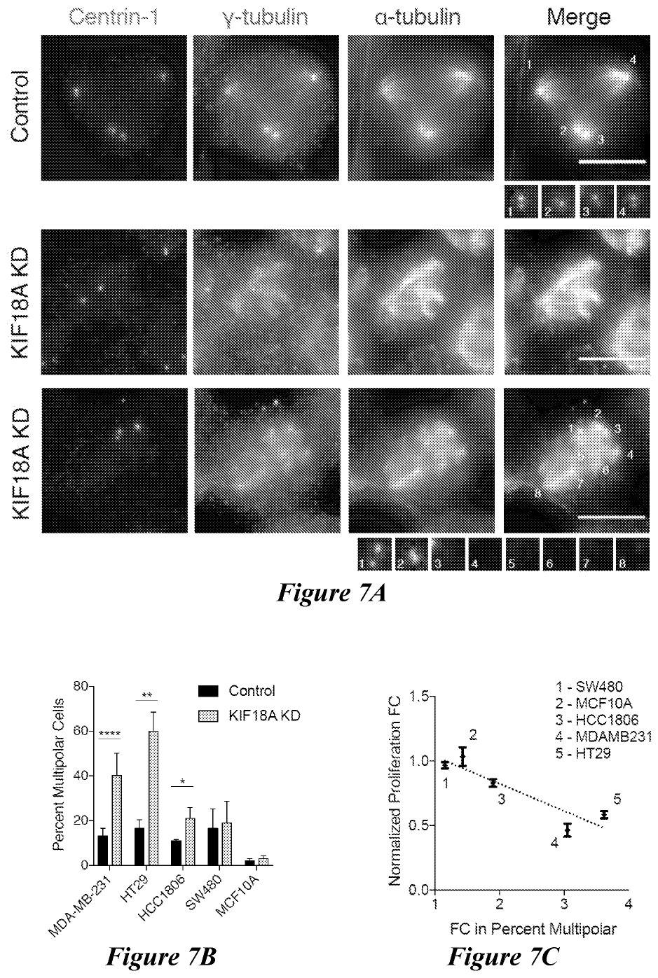

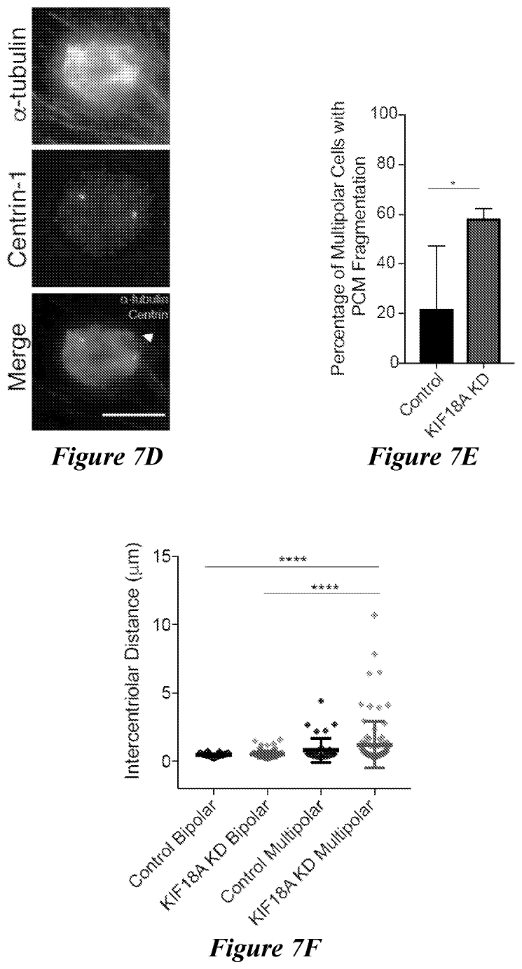

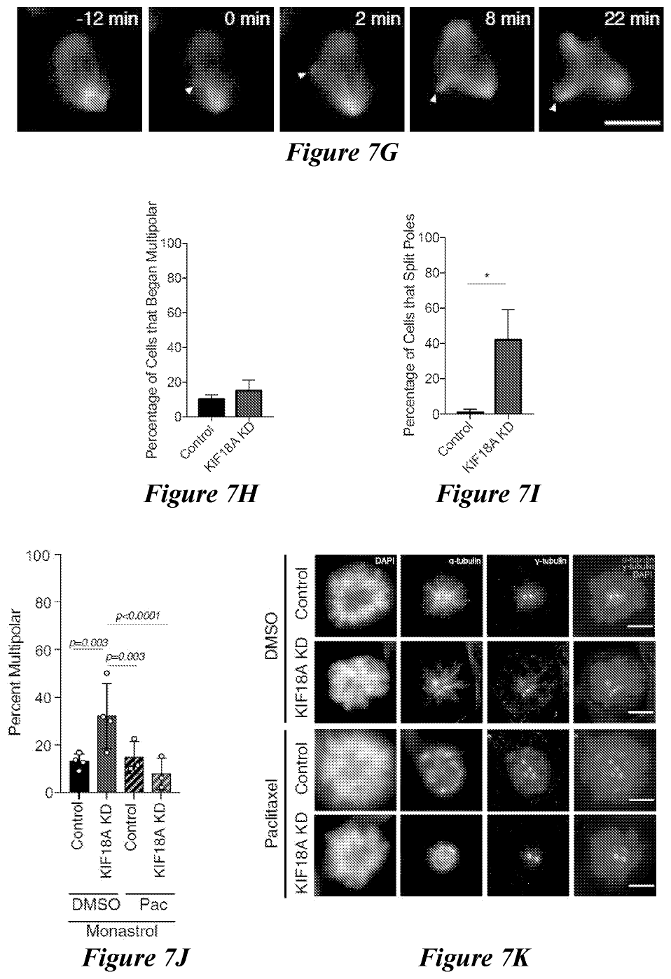

Analyses of mitotic spindles in KIF18A KD cells revealed that KIF18A-dependent CIN lines display a significant increase in multipolar spindles compared to non-KIF18A-dependent cell lines (FIGS. 7A-7B). Interestingly, the fold-increase in multipolar spindles following KIF18A KD was inversely proportional to the fold-decrease in proliferation for each cell type (FIG. 7C). These data indicate that mitotic spindle assembly is abnormal in KIF18A-dependent CIN cells.

Loss of KIF18A function could lead to multipolar spindles by promoting centrosome amplification, cytokinesis failure, centriole disengagement, or pericentriolar material (PCM) fragmentation (Maiato & Logarinho, “Mitotic Spindle Multipolarity Without Centrosome Amplification,” Nat Cell Biol 16:386-394 (2014), which is hereby incorporated by reference in its entirety). To distinguish among these mechanisms, the number and organization of centrioles within multipolar spindles in MDA-MB-231 cells were analyzed (FIGS. 7D-7F). The majority of spindles (˜75%) in both control and KIF18A KD cells contained four centrioles, indicating that centrosome amplification and cytokinesis failure do not significantly contribute to spindle defects in KIF18A KD cells. The distance between paired centrioles was increased in multipolar KIF18A KD cells compared to those in bipolar spindles but was comparable to that measured in multipolar spindles treated with control siRNA (FIG. 7F). However, ˜60% of multipolar KIF18A KD cells exhibited γ-tubulin containing microtubule organizing centers without centrioles (FIG. 7E). Furthermore, live imaging of KIF18A-depleted MDA-MB-231 cells labeled with siR-tubulin revealed an increase in spindle pole fragmentation events but not the number of cells entering mitosis with multiple poles compared to control siRNA treated cells (FIGS. 7G-7I). These data suggest that KIF18A KD primarily leads to multipolar spindles by inducing PCM fragmentation.

Altered microtubule dynamics in KIF18A KD cells could lead to centrosome fragmentation by disrupting the balance of pushing and pulling forces within bipolar spindles. To test this idea, the number of γ-tubulin foci in MDA-MB-231 cells treated with the KIF11 inhibitor monastrol was assayed. Monastrol induces monopolar spindles by preventing KIF11-dependent antiparallel microtubule sliding forces (Kapoor et al., “Probing Spindle Assembly Mechanisms with Monastrol, a Small Molecule Inhibitor of the Mitotic Kinesin Eg5.” J. Cell Biol. 150:975-988 (2000), which is hereby incorporated by reference in its entirety). Centrosome fragmentation still occurred in monopolar KIF18A KD cells and could be reduced by co-treatment with paclitaxel (FIGS. 7J and 7K). Live imaging of monastrol treated cells expressing RFP-pericentrin to label centrosomes revealed that centrosomes begin intact in monopolar KIF18A KD cells and subsequently fragment. These data suggest that neither bipolar spindles nor the forces generated via KIF11-dependent microtubule sliding are required for centrosome fragmentation in the absence of KIF18A.

KIF18A KD Induces Multipolar Spindles in CIN Cells Independently of Mitotic Delay

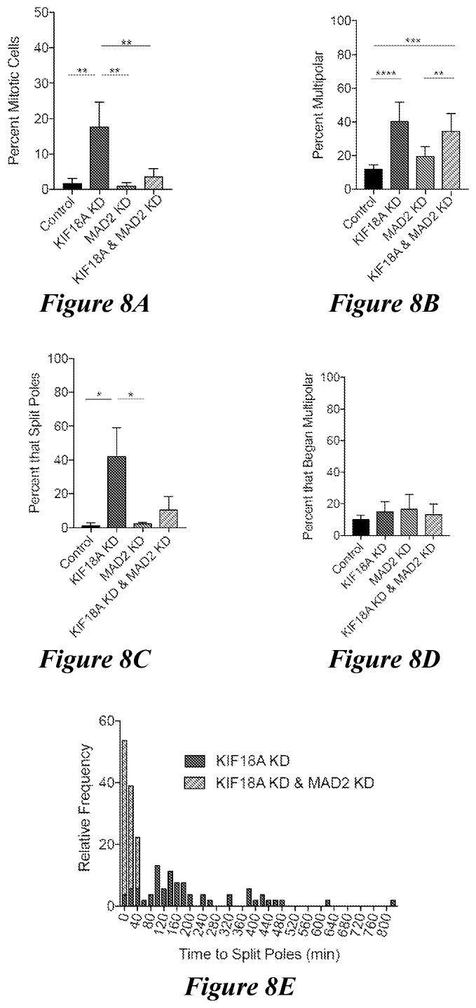

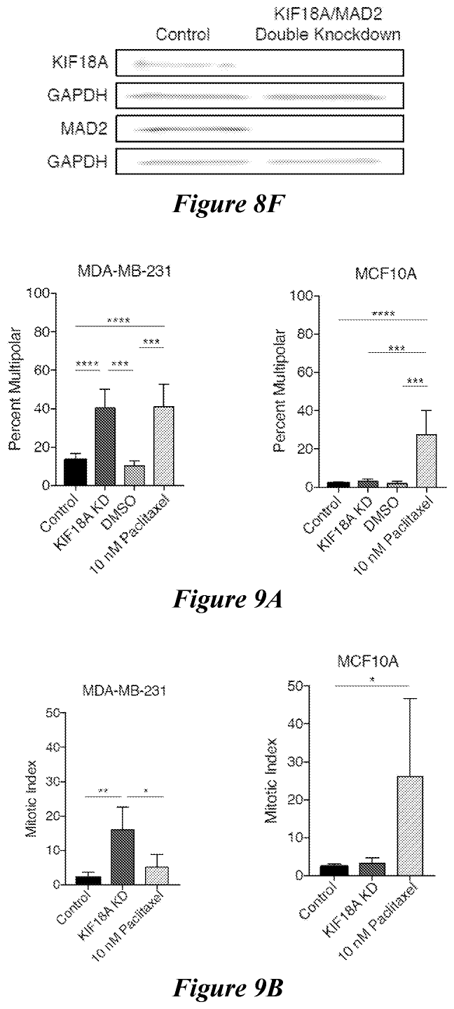

The fragmentation of centrosomes and formation of multipolar spindles following KIF18A KD could result from abnormal spindle forces caused by altered microtubule dynamics or as a secondary effect of an extended mitotic delay (Maiato & Logarinho, “Mitotic Spindle Multipolarity Without Centrosome Amplification,” Nat Cell Biol 16:386-394 (2014), which is hereby incorporated by reference in its entirety). To determine if a mitotic delay is required for multipolar spindle formation following KIF18A KD, spindle morphology was analyzed in MDA-MB-231 cells depleted of both KIF18A and MAD2, which is required for spindle assembly checkpoint-dependent mitotic arrest (Gorbsky et al., “Microinjection of Antibody to Mad2 Protein into Mammalian Cells in Mitosis Induces Premature Anaphase.” J Cell Biol 141:1193-1205 (1998), which is hereby incorporated by reference in its entirety). KIF18A/MAD2 KD cells displayed a reduced mitotic index but a similar level of multipolar spindles compared to KIF18A KD cells (FIGS. 8A-8B). Spindle pole splitting in live cells occurred at a range of times after mitotic entry in KIF18A KD cells and at times shortly after NEB in KIF18A/MAD2 KD cells (FIGS. 8C-8E). The significant decrease in multipolar KIF18A/MAD2 KD cells compared to KIF18A KD alone observed during live imaging may be explained by the limitations inherent to the identification of multipolar spindles in live assays, as poles must split sufficiently far apart to be completely separated in this case. Therefore, the live approach is likely to underestimate the actual time to splitting and percentage of multipolar spindles, especially in cells that exit mitosis quickly. Taken together, these data suggest that loss of KIF18A leads to spindle pole fragmentation in CIN cells and that this defect does not require, but may be enhanced by, a mitotic delay.

CIN Cells Display Increased Sensitivity for KIF18A KD Over Diploid Cells as Compared to Paclitaxel

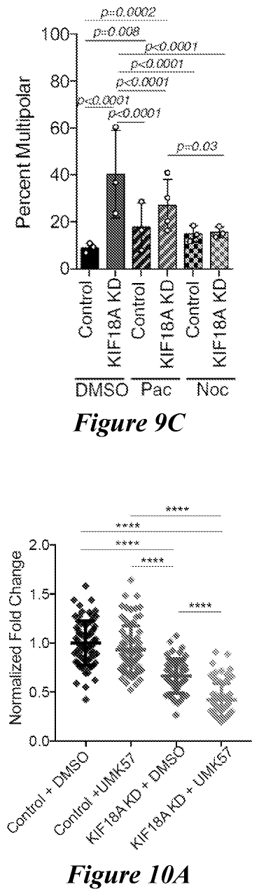

The mitotic delay and multipolar spindles caused by KIF18A KD in some tumor cells are similar to those observed following treatment with clinically relevant doses of paclitaxel (Zasadil et al., “Cytotoxicity of Paclitaxel in Breast Cancer is Due to Chromosome Missegregation on Multipolar Spindles,” Sci Transl Med 6:229ra43-229ra43 (2014), which is hereby incorporated by reference in its entirety). This is somewhat unexpected, as the two treatments have opposite effects on spindle microtubules. Microtubules grow faster and longer in the absence of KIF18A's microtubule growth suppressing function, while paclitaxel stabilizes microtubules and slows dynamic instability (Schiff et al., “Promotion of Microtubule Assembly In Vitro by Taxol,” Nature 277:665-667 (1979); Du et al., “The Kinesin-8 Kif18A Dampens Microtubule Plus-end Dynamics,” Curr Biol 20:374-380 (2010); Stumpff et al., “A Tethering Mechanism Controls the Processivity and Kinetochore-microtubule Plus-end Enrichment of the Kinesin-8 Kif18A,” Mol Cell 43:764-775 (2011), which are hereby incorporated by reference in their entirety). Interestingly, it was found that KIF18A KD and 10 nM paclitaxel produced similar mitotic defects in MDA-MB-231 cells, but only paclitaxel increased the mitotic index and multipolar spindles in diploid MCF10A cells (FIGS. 9A-9B). These data suggest that CIN tumor cells may be particularly sensitive to the increased microtubule dynamics that occur following KIF18A loss of function, while diploid cells are minimally affected by this change.

KIF18A functions to suppress microtubule growth in mitotic spindles (Stumpff et al., “The Kinesin-8 Motor, Kif18A, Suppresses Kinetochore Movements to Control Mitotic Chromosome Alignment,” Dev. Cell 14(2): 252-262 (2008); Zhu et al., “Functional Analysis of Human Microtubule-based Motor Proteins, the Kinesins and Dyneins, in Mitosis/Cytokinesis Using RNA Interference,” Mol Biol Cell 16:3187-3199 (2005), which are hereby incorporated by reference in their entirety), suggesting that abnormal microtubule dynamics in KIF18A KD cells may contribute to centersome fragmentation. This was tested by reducing microtubule polymerization or depolymerizing microtubules completely via the treatment of KIF18A KD MDA-MB-231 cells with 20 nM paclitaxel or 5 μM nocodazole, respectively (Yvon et al., “Taxol Suppresses Dynamics of Individual Microtubules in Living Human Tumor Cells,” Mol. Biol. Cell 10: 947-959 (1999); Jordan et al., “Effects of Vinblastine, Podophyllotoxin and Nocodazole on Mitotic Spindles. Implications for the Role of Microtubule Dynamics in Mitosis,” J. Cell Sci. 102:401-416 (1992), which are hereby incorporated by reference in their entirety). KIF18A KD cells treated with either paclitaxel or nocodazole for 3 h before fixation displayed significantly fewer multipolar spindles than KIF18A KD cells treated with DMSO (FIG. 9C). These data indicate that dynamic microtubules are required for KIF18A KD induced centrosome fragmentation.

The CIN Cell-Specific Effects of KIF18A KD are Enhanced by a Small Molecule Activator for Microtubule Depolymerization

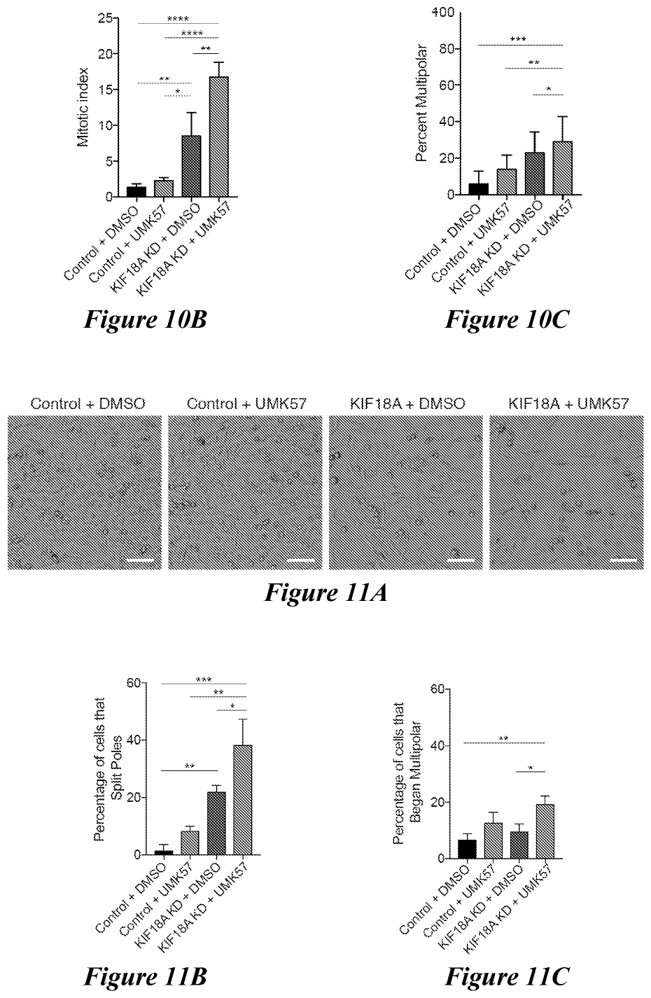

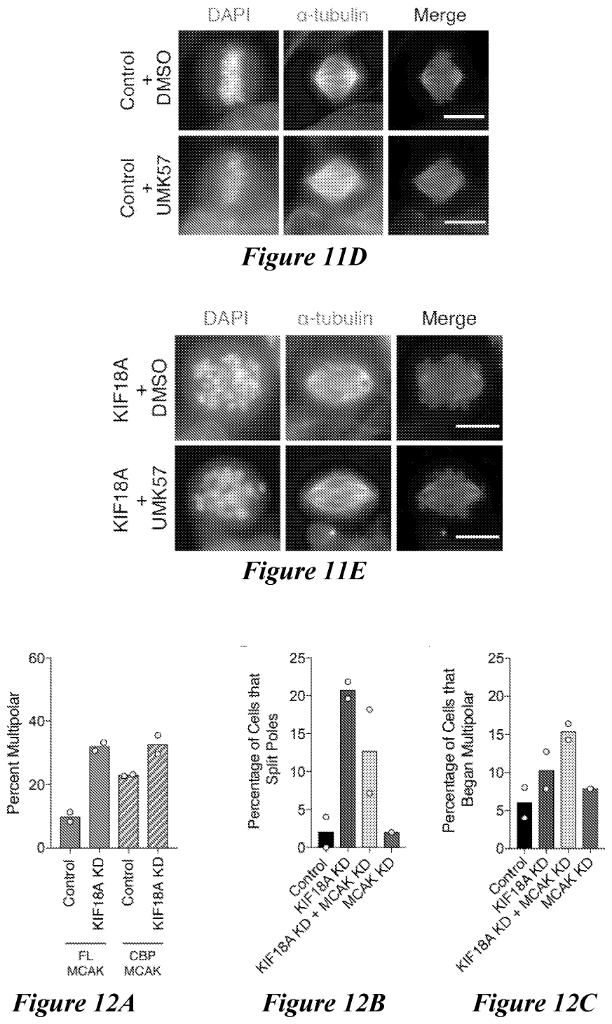

KIF18A suppresses the dynamics of kinetochore microtubules to promote chromosome alignment and decreases kinetochore microtubule turnover (Stumpff et al., “The Kinesin-8 Motor Kif18A Suppresses Kinetochore Movements to Control Mitotic Chromosome Alignment,” Dev Cell 14:252-262 (2008); Wordeman et al., “Divergent Microtubule Assembly Rates After Short- Versus Long-term Loss of End-modulating Kinesins,” Mol Biol Cell 27:1300-1309 (2016), which are hereby incorporated by reference in their entirety). Increased kinetochore microtubule turnover may contribute to the prolonged mitotic delays and destabilized spindles observed in KIF18A KD CIN cells. This was tested by treating cells with a small molecule (UMK57) that promotes kinetochore microtubule turnover by increasing the activity of the depolymerizing kinesin MCAK (Orr et al., “Adaptive Resistance to an Inhibitor of Chromosomal Instability in Human Cancer Cells,” Cell Reports 17:1755-1763 (2016), which is hereby incorporated by reference in its entirety). Treatment of KIF18A-depleted MDA-MB-231 cells with UMK57 (500 nM) decreased proliferation and increased both the mitotic index and percentage of multipolar spindles beyond what is seen in KIF18A KD cells treated with DMSO (FIGS. 10A-10C and FIG. 11A). The same concentration of UMK57 had no impact on the proliferation of control siRNA-treated cells (FIGS. 11A-11C). Furthermore, live cell imaging of siR-tubulin showed that KIF18A KD cells treated with UMK57 displayed increased spindle pole splitting without an obvious change in chromosome alignment defects in bipolar spindles (FIGS. 11B-11E). UMK57 treatment of KIF18A KD cells also led to a small but significant increase in multipolar spindles, and this effect was replicated in cells with increased global MCAK/KIF2C activity, due to overexpression of mCherry-MCAK, or increased MCAK/KIF2C activity at centromeres, due to expression of mCherry-CPB-MCAK (FIGS. 10C and 12A) (Wordeman et al., “MCAK Facilitates Chromosome movement by promoting kinetochore microtubule turnover,” J. Cell. Biol. 179:869-879 (2007), which is hereby incorporated by reference in its entirety). Additionally, in live cells labeled with siR-tublin, co-depletion of both KIF18A and KIF2C reduced multipolar spindle formation compared to depletion of KIF18A alone, while KIF18A KD cells treated with UMK57 displayed increased spindle pole fragmentation (FIGS. 11B, 11C, 12B and 12C). These data indicate that loss of KIF18A function and increased MCAK function synergistically disrupt mitotic progression and spindle bipolarity in CIN cells.

### CONCLUSIONS

The data presented herein support a model in which the altered microtubule dynamics in mitotic CIN cells make them particularly dependent on KIF18A to reduce kinetochore microtubule turnover, which in turn is required to maintain spindle bipolarity and promote mitotic progression. Importantly, it was found that KIF18A is not required for mitosis or proliferation of near-diploid cells. These results are consistent with previous observations that loss of KIF18A leads to spindle assembly checkpoint-dependent delays in cancer cells but not in diploid somatic cells (Mayr et al., “The Human Kinesin Kif18A is a Motile Microtubule Depolymerase Essential for Chromosome Congression,” Curr Biol 17:488-498 (2007); Czechanski et al., “Kif18a is Specifically Required for Mitotic Progression During Germ Line Development,” Dev Biol 402:253-262 (2015); Fonseca et al., “Mitotic Chromosome Alignment Ensures Mitotic Fidelity by Promoting Interchromosomal Compaction During Anaphase,” J Cell Biol 218:1086-1088 (2019); Janssen et al., “Loss of Kif18A Results in Spindle Assembly Checkpoint Activation at Microtubule-Attached Kinetochores,” Curr Biol 28(17):2685-2696 (2018); Edzuka & Goshima, “Drosophila Kinesin-8 Stabilizes the Kinetochore-microtubule Interaction,” J Cell Biol 5:jcb.201807077 (2018); Zhu et al., “Functional Analysis of Human Microtubule-based Motor Proteins, the Kinesins and Dyneins, in Mitosis/Cytokinesis Using RNA Interference,” Mol Biol Cell 16:3187-3199 (2005); which are hereby incorporated by reference in their entirety). KIF18A is also largely dispensable for proliferation of diploid somatic cells in vivo but is necessary for tumor growth. Kif18a mutant mice display an early growth delay and germline development defects but are viable (Czechanski et al., “Kif18a is Specifically Required for Mitotic Progression During Germ Line Development,” Dev Biol 402:253-262 (2015); Liu et al., “Germinal Cell Aplasia in Kif18a Mutant Male Mice Due to Impaired Chromosome Congression and Dysregulated BubR1 and CENP-E,” Genes Cancer 1:26-39 (2010), which are hereby incorporated by reference in their entirety). However, the growth of both induced CRC and xenografted TNBC tumors in mouse models are dependent on KIF18A (Zhu et al., “Targeted Deletion of Kif18a Protects from Colitis-associated Colorectal (CAC) Tumors in Mice Through Impairing Akt Phosphorylation,” Biochem Bioph Res Co 438:97-102 (2013); Zhang et al., “Kif18A is Involved in Human Breast Carcinogenesis,” Carcinogenesis 31:1676-1684 (2010), which are hereby incorporated by reference in their entirety). Thus, KIF18A may be an effective target to specifically inhibit the growth of CIN tumor cells, while inducing relatively low toxicity in somatic, diploid cells.

These data raise the important question of why CIN cells would depend more on KIF18A for successful mitosis than normal cells. CIN cells exhibit increased rates of spindle microtubule polymerization and altered turnover of kinetochore microtubules (Bakhoum et al., “Deviant Kinetochore Microtubule Dynamics Underlie Chromosomal Instability,” Curr Biol 19:1937-1942 (2009); Ertych et al., “Increased Microtubule Assembly Rates Influence Chromosomal Instability in Colorectal Cancer Cells,” Nat Cell Biol 16:779-791 (2014), which are hereby incorporated by reference in their entirety), which may confer an enhanced dependence on KIF18A's function to suppress the growth of kinetochore microtubules. The results presented in the present application indicate that in the absence of KIF18A activity, maintenance of kinetochore microtubule attachments and the balance of forces within the spindle are defective in CIN cells, subsequently leading to mitotic arrest and centrosome fragmentation. Previous observations that KIF18A reduces the turnover of microtubules from kinetochores and is required to generate tension between paired kinetochores are consistent with this interpretation (Mayr et al., “The Human Kinesin Kif18A is a Motile Microtubule Depolymerase Essential for Chromosome Congression,” Curr Biol 17:488-498 (2007); Stumpff et al., “The Kinesin-8 Motor Kif18A Suppresses Kinetochore Movements to Control Mitotic Chromosome Alignment,” Dev Cell 14:252-262 (2008); Wordeman et al., “Divergent Microtubule Assembly Rates After Short-Versus Long-term Loss of End-modulating Kinesins,” Mol Biol Cell 27:1300-1309 (2016); Stumpff et al., “Kif18A and Chromokinesins Confine Centromere Movements Via Microtubule Growth Suppression and Spatial Control of Kinetochore Tension,” Dev Cell 22:1017-1029 (2012), which are hereby incorporated by reference in their entirety). In addition, KIF18A KD cells that do complete mitosis form micronuclei as a result of chromosome alignment defects (Fonseca et al., “Mitotic Chromosome Alignment Ensures Mitotic Fidelity by Promoting Interchromosomal Compaction During Anaphase,” J Cell Biol 218:1086-1088 (2019), which is hereby incorporated by reference in its entirety). The frequency of micronucleus formation in KIF18A-depleted cells is enhanced by elevated chromosome number, and therefore, could also contribute to the specific reduction in proliferation observed in aneuploid cells.

The tests of the effects of other kinesins that control spindle microtubule dynamics and chromosome movements suggest the specific dependence of CIN cells on KIF18A is unique among mitotic kinesins. Other mitotic kinesins are either not required for division of CIN cells or are required for division of both diploid and CIN cells. In agreement, two recent, large-scale bioinformatics studies identified Kif18A, but not other kinesins, as a gene specifically required for the growth of aneuploid cells. These data indicate the broad implications of our results and strongly support further investigation into therapeutically relevant mitotic vulnerabilities specific to CIN tumor cells.

Although preferred embodiments have been depicted and described in detail herein, it will be apparent to those skilled in the relevant art that various modifications, additions, substitutions, and the like can be made without departing from the spirit of the application and these are therefore considered to be within the scope of the application as defined in the claims which follow.

What is claimed:

1. A method of inhibiting proliferation of chromosome instable cancer cells, said method comprising:
administering, to a population of cancer cells comprising chromosome instable cancer cells, an inhibitor of Kinesin Family Member 18A (KIF18A) at a dosage effective to inhibit proliferation of said chromosome instable cancer cells.

2. The method of claim 1, wherein the KIF18A inhibitor comprises a compound of Formula I 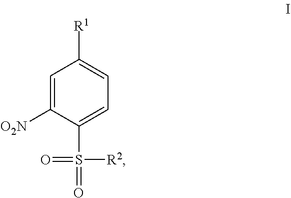
or a derivative thereof,
wherein R¹ is selected from NO₂, F, Cl, CF₃, and H, and R² is selected from phenyl or 2-thiophene.

3. The method of claim 1, wherein said administering further comprises:
administering to the population of cells, in conjunction with the KIF18A inhibitor, an agent that promotes microtubule turnover.

4. The method of claim 3, wherein the agent that promotes microtubule turnover is an agent that enhances mitotic centromere-associated kinesin (MCAK) activity.

5. The method of claim 4, wherein the agent that enhances MCAK activity is a compound of Formula III 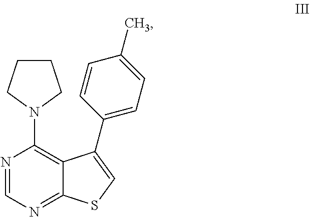 [SMILES: Cc1ccc(-c2csc3ncnc(N4CCCC4)c23)cc1]
or a derivative thereof.

6. The method of claim 3, wherein the agent that promotes microtubule turnover is a microtubule destabilizing agent.

7. The method of claim 6, wherein the microtubule destabilizing agent is selected from the group consisting of nocodazole, vincristine, vinblastine, vinorelbine, vindesine, vinflunine, colchicine, and Erubulin mesylate.

8. The method any one of claims 3-7, wherein the KIF18A inhibitor and agent that promotes microtubule turnover are administered concurrently.

9. The method any one of claims 3-7, wherein the KIF18A inhibitor and agent that promotes microtubule turnover are administered sequentially.

10. The method of any one of claims 1-9, wherein the cancer cells are mammalian cancer cells.

11. The method of any one of claims 1-9, wherein the cancer cells are human cancer cells.

12. The method any one of claims 1-11, wherein the population of cancer cells comprising chromosome instable cancer cells is selected from a population of breast cancer cells, bladder cancer cells, colorectal cancer cells, prostate cancer cells, cervical cancer cells, endometrial cancer cells, lung cancer cells, liver cancer cells, high hyperdiploid acute lymphoblastic leukemia cells, ovarian cancer cells, and glioblastoma cells.

13. The method of claim 12, wherein the population of breast cancer cells is a population of triple negative breast cancer cells.

14. The method of claim 12, wherein the population of cancer cells is a population of chromosome instable colorectal cancer cells.

15. A method of treating cancer in a subject, said method comprising:
administering to a subject having cancer, wherein said cancer is characterized by chromosomal instability, an inhibitor of Kinesin Family Member 18A (KIF18A) at a dosage effective to treat the cancer in the subject.

16. The method of claim 15, wherein the KIF18A inhibitor comprises a compound of Formula I 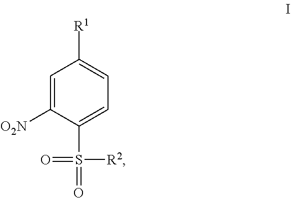
or a derivative thereof,
wherein R¹ is selected from NO₂, F, Cl, CF₃, and H, and R² is selected from phenyl or 2-thiophene.

17. The method of claim 15 or claim 16, wherein said administering further comprises:
administering an agent that promotes microtubule turnover to the subject in conjunction with the KIF18A inhibitor.

18. The method of claim 17, wherein the agent that promotes microtubule turnover is an agent that enhances mitotic centromere-associated kinesin (MCAK) activity.

19. The method of claim 18, wherein the agent that enhances MCAK activity is a compound of Formula III 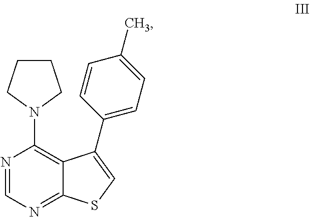 [SMILES: Cc1ccc(-c2csc3ncnc(N4CCCC4)c23)cc1]
or a derivative thereof.

20. The method of claim 17, wherein the agent that promotes microtubule turnover is a microtubule destabilizing agent.

21. The method of claim 20, wherein the microtubule destabilizing agent is selected from the group consisting of nocodazole, vincristine, vinblastine, vinorelbine, vindesine, vinflunine, colchicine, and Erubulin mesylate.

22. The method of any one of claims 17-21, wherein the KIF18A inhibitor and agent that promotes microtubule turnover are administered concurrently.

23. The method of any one of claims 17-21, wherein the KIF18A inhibitor and agent that promotes microtubule turnover are administered sequentially.

24. The method of any one of claims 15-23, wherein said administering further comprises:
administering a cyclin-dependent kinase (CDK) inhibitor to said subject.

25. The method of claim 24, wherein the CDK inhibitor is a CDK 4 and/or CDK6 inhibitor.

26. The method of claim 25, wherein the CDK inhibitor is selected from palbociclib, ribociclib, and abemaciclib.

27. The method of any one of claims 15-26, wherein said cancer is a chromosome instable form of breast cancer, bladder cancer, colorectal cancer, prostate cancer, cervical cancer, lung cancer, liver cancer, endometrial cancer, high hyperdiploid acute lymphoblastic leukemia, ovarian cancer, and glioblastoma.

28. The method of claim 27, wherein the cancer is triple negative breast cancer.

29. The method of claim 27, wherein the cancer is a chromosome instable form of colorectal cancer.

30. The method of any one claims 15-29, wherein said subject is a human.

31. A combination therapeutic comprising:
an inhibitor of Kinesin Family Member 18A (KIF18A); and
an agent that promotes microtubule turnover.

32. The combination therapeutic of claim 29, wherein the KIF18A inhibitor comprises a compound of Formula I 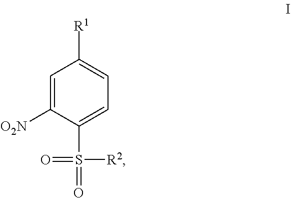
or a derivative thereof,
wherein R¹ is selected from NO₂, F, Cl, CF₃, and H, and R² is selected from phenyl or 2-thiophene.

33. The combination therapeutic of claim 31 or claim 32, wherein the agent that promotes microtubule turnover is an agent that enhances MCAK activity.

34. The combination therapeutic of claim 33, wherein the agent that enhances MCAK activity is a compound of Formula III 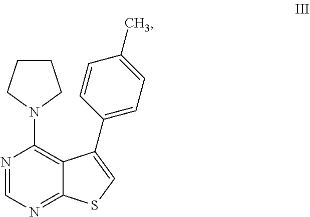 [SMILES: Cc1ccc(-c2csc3ncnc(N4CCCC4)c23)cc1]
or a derivative thereof.

35. The combination therapeutic of claim 31 or claim 32, wherein the agent that promotes microtubule turnover is a microtubule destabilizing agent.

36. The combination therapeutic of claim 35, wherein the microtubule destabilizing agent is selected from the group consisting of nocodazole, vincristine, vinblastine, vinorelbine, vindesine, vinflunine, colchicine, and Erubulin mesylate.

37. The combination therapeutic of any one or claims 31-36, wherein the KIF18A inhibitor and the agent that promotes microtubule turnover are formulated together in a single pharmaceutical composition.

38. The combination therapeutic of any one of claims 31-36, wherein the KIF18A inhibitor and the agent that promotes microtubule turnover are formulated as separate pharmaceutical compositions.

39. The combination therapeutic of claim 31-36 further comprising:
a cyclin-dependent kinase (CDK) inhibitor.

40. The combination therapeutic of claim 39, wherein the CDK inhibitor is a CDK 4 and/or CDK6 inhibitor.

41. The combination therapeutic of claim 40, wherein the CDK inhibitor is selected from palbociclib, ribociclib, and abemaciclib.

42. A combination therapeutic comprising:
an inhibitor of Kinesin Family Member 18A (KIF18A); and
a cyclin-dependent kinase (CDK) inhibitor.

43. The combination therapeutic of claim 42, wherein the KIF18A inhibitor comprises a compound of Formula I 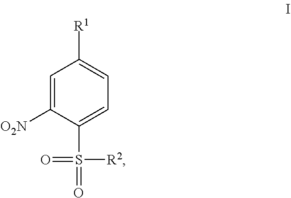
or a derivative thereof,
wherein R¹ is selected from NO₂, F, Cl, CF₃, and H, and R² is selected from phenyl or 2-thiophene.

44. The combination of claim 42 or claim 43, wherein the CDK inhibitor is a CDK 4 and/or CDK6 inhibitor.

45. The combination of claim 44, wherein the CDK inhibitor is selected from palbociclib, ribociclib, and abemaciclib.

46. The combination therapeutic of any one of claims 42-45, wherein the KIF18A inhibitor and the CDK inhibitor are formulated together in a single pharmaceutical composition.

47. The combination therapeutic of any one of claims 42-45, wherein the KIF18A inhibitor and the CDK inhibitor are formulated as separate pharmaceutical compositions.
